(19) United States
(12) Patent Application Publication
(10) Pub. No.: US 2022/0289856 A1
(43) Pub. Date: Sep. 15, 2022

(54) MULTIMERIC BISPECIFIC ANTI-CD123 BINDING MOLECULES AND USES THEREOF

(71) Applicant: IGM Biosciences, Inc., Mountain View, CA (US)

(72) Inventors: Manal AMOURY, San Jose, CA (US); Angus SINCLAIR, San Francisco, CA (US); Bruce KEYT, Hillsborough, CA (US)

(21) Appl. No.: 17/635,490

(22) PCT Filed: Aug. 14, 2020
(86) PCT No.: PCT/US2020/046335

§ 371 Date: Feb. 15, 2022

Related U.S. Application Data
(60) Provisional application No. 62/888,475, filed on Aug. 17, 2019.
(60) Provisional application No. 62/888,702, filed on Aug. 19, 2019.

(51) Int. Cl. C07K 16/28; A61P 35/02

(52) U.S. Cl. CPC C07K 16/2866; C07K 16/2809; A61P 35/02; C07K 2317/31; C07K 2317/622; C07K 2317/64; A61K 39/00

(57) ABSTRACT

This disclosure provides multivalent, bispecific, anti-CD123 binding molecule comprising a modified J-chain that specifically binds to an immune effector cell. Also provided are polynucleotides encoding the binding molecule or subunits thereof and vectors and host cell comprising said polynucleotides. This disclosure further provides methods for producing and/or using a multivalent, bispecific, anti-CD123 binding molecule comprising a modified J-chain that specifically binds to an immune effector cell.

### CROSS-REFERENCE TO RELATED APPLICATIONS

This application claims the benefit of U.S. Provisional Patent Application Ser. No. 62/888,475, filed Aug. 17, 2019 and U.S. Provisional Patent Application Ser. No. 62/888,702, filed Aug. 19, 2019, which are each incorporated herein by reference in their entireties.

### SEQUENCE LISTING

The instant application contains a Sequence Listing which has been submitted electronically in ASCII format and is hereby incorporated by reference in its entirety. The ASCII copy was created on Aug. 13, 2020, is named 027WO1-Sequence-Listing, and is 204,738 bytes in size.

### BACKGROUND

Acute Myeloid Leukemia (AML) is the leading cause of leukemia mortality in the United States, with >20,000 new patients per year with a 5-year survival of less than 30%, with the survival rate decreasing to 10% in patients over 60 years old (National Cancer Institute Surveillance, Epidemiology and End-Result Program (SEER) data; Oran and Weisdorf 2012, Haematologica 97(12) 1916). Few advances have been made in the treatment of AML patients for the past 40 years, and current treatment options primarily consist of intense chemotherapy and stem cell transplantation (Luppi et al. 2018, Cancers 10, 429). Several approaches have been taken to target cell surface molecules on AML cells to direct T cells to engage and kill AML cells. One such surface molecule is CD123 (also known as IL-3 receptor alpha chain or IL-3Rα) that is expressed in >90% of AML patients on leukemic cells as well as leukemic stem cells, a cell type which is often responsible for disease relapse after therapy (Kovtun et al. 2018, Blood Advances 2(8) 848; Xie et al 2017, Blood Cancer Journal 7, e567). In addition, CD123 is highly expressed in patients that have genetic mutations associated with a very poor prognosis, such as FLT3 (Xie et al 2017, Blood Cancer Journal 7, e567). The amino acid sequences of two human isoforms of CD123 are presented as SEQ ID NO: 28 (isoform 1, mature protein: approximately amino acids 23 to 378 of SEQ ID NO: 28) and SEQ ID NO: 29 (isoform 2, mature protein: approximately amino acids 23 to 300 of SEQ ID NO: 29), the cynomolgus monkey CD123 amino acid sequence is presented as SEQ ID NO: 30 (about 87% identical to human isoform 1; mature protein: approximately amino acids 23 to 378 of SEQ ID NO: 30), and the mouse CD123 amino acid sequence is presented as SEQ ID NO: 31 (about 30% identical to human isoform 1; mature protein: approximately amino acids 17 to 396 of SEQ ID NO: 31).

CD123 is a clinically validated target for some hematological malignancies as evidence by the FDA approval of a recombinant IL-3 cytokine conjugated with diphtheria toxin for the treatment of blastic plasmacytoid dendritic cell neoplasms (Pemmaraju et al 2019, NEJM 380:1628). This and other CD123 targeting agents are being tested in preclinical and clinical trials. Early Phase 1 clinical studies have been conducted with CD123×CD3 bispecific antibodies by Xencor (XmAb14045—IgG based), Macrogenics (flotetuzumab—DART) and Jansen (JNJ-63709178—duobody). Though early signs of clinical efficacy have been reported in some of these patients, severe cytokine release syndrome and some patient deaths have also been observed with this class of bispecific drugs (Ravandi et al 2018 Blood 132:763; Jacobs et al 2018, Blood 132:2738; Uy et al 2018, Blood 132:764). Cytokine release syndrome (or CRS) is characterized by fever, hypotension, blood coagulation abnormalities and capillary leak which can be life threatening and such findings are also associated with other T cell engaging approaches, including CAR-Ts and BiTEs (Teachley et al 2016, Cancer Discovery 6(6) 664; Hay et al 2017, Blood 130(21) 2295). These adverse safety events related to cytokine release tend to constitute dose limiting toxicities of IgG based CD3 engaging bispecific antibodies and manifest as challenges to the safe, efficient and tolerable administration of such agents and potentially to the ability to optimize efficacy of these therapeutic agents due to the resulting limitations to dosing.

Antibodies and antibody-like molecules that can multimerize, such as IgA and IgM antibodies, have emerged as promising drug candidates in the fields of, e.g., immuno-oncology and infectious diseases allowing for improved specificity, improved avidity, and the ability to bind to multiple binding targets. See, e.g., U.S. Pat. Nos. 9,951,134, 9,938,347, and 10,618,978, U.S. Patent Application Publication No. US 2019-0100597, US 2019-0185570, and PCT Publication Nos. WO 2016/154593, WO 2016/168758, WO 2018/017888, WO 2018/017763, WO 2018/017889, WO 2018/017761, and WO 2019/169314, the contents of which are incorporated herein by reference in their entireties.

There remains a need to target CD123-expressing AML cells and induce T-cell mediated killing of those cells, while minimizing CRS. We have evaluated whether targeting CD123 with our CD3 bispecific IgM technology will not only effectively target CD123 expressing AML tumor cells for T cell mediated cytotoxicity, but will also produce responses with a favorable safety profile for the cytokine release syndrome that has sometimes been severe in patients treated with IgG based CD123×CD3 bispecific antibodies. In addition, the high avidity binding of IgM antibodies may allow our CD123×CD3 bispecific IgM to target tumor cells that express relatively lower levels of cell surface expression of CD123, as compared with IgG based bispecific antibodies.

### SUMMARY

This disclosure provides a multimeric, bispecific or multispecific binding molecule including two or five bivalent binding units and a modified J-chain, where the modified J-chain includes a wild-type J-chain or a functional fragment or variant thereof and a J-chain-associated antigen-binding domain that specifically binds to an immune effector cell. Each binding unit includes two antibody heavy chains, each including an IgA, IgA-like, IgM, or IgM-like heavy chain constant region or multimerizing fragment thereof and at least a heavy chain variable region (VH) portion of a binding unit-associated antigen-binding domain, where at least three of the binding unit-associated antigen-binding domains specifically bind to CD123, and where the binding molecule can induce immune effector cell-dependent killing of cells expressing CD123.

In certain embodiments, the modified J-chain includes a variant J-chain or fragment thereof including one or more single amino acid substitutions, deletions, or insertions relative to a wild-type J-chain that can affect serum half-life of the binding molecule, such that the binding molecule exhibits an increased serum half-life upon administration to an animal relative to a reference binding molecule that is identical except for the one or more single amino acid substitutions, deletions, or insertions in the J-chain, and is administered in the same way to the same animal species. In certain embodiments, the modified J-chain includes an amino acid substitution at the amino acid position corresponding to amino acid Y102 of the mature wild-type human J-chain (SEQ ID NO: 2). In certain embodiments, the amino acid corresponding to Y102 of SEQ ID NO: 2 can be substituted with alanine (A), serine (S), or arginine (R). In certain embodiments, the amino acid corresponding to Y102 of SEQ ID NO: 2 can be substituted with alanine (A). In certain embodiments, the J-chain is a variant human J-chain and includes the amino acid sequence SEQ ID NO: 3 (“J*”).

In certain embodiments, the J-chain-associated antigen-binding domain includes an antibody single chain Fv (scFv) fragment fused or chemically conjugated to the J-chain or fragment or variant thereof. For example, the scFv fragment can be fused to the J-chain via a peptide linker. In certain embodiments, the scFv fragment can be fused to the N-terminus of the J-chain or fragment or variant thereof, the C-terminus of the J-chain or fragment or variant thereof, or scFv fragments can be fused to both the N-terminus and C-terminus of the J-chain or fragment or variant thereof.

In certain embodiments, the immune effector cell is a T cell or an NK cell. In those embodiments where the immune effector cell is a T cell, the scFv fragment, in certain embodiments, can specifically bind to CD3. In certain embodiments, the T cell is a CD8+ cytotoxic T cell.

In certain embodiments, the scFv fragment comprises a heavy chain variable region (VH) and a light chain variable region (VL), where the VH comprises VH complementarity-determining regions VHCDR1, VHCDR2, and VHCDR3 and the VL comprises VL complementarity-determining regions VLCDR1, VLCDR2, and VLCDR3, where (a) the VHCDR1, VHCDR2, and VHCDR3 comprise the amino acid sequences SEQ ID NO: 5, SEQ ID NO: 6, and SEQ ID NO: 7 with zero, one, or two amino acid substitutions, respectively, and the VLCDR1, VLCDR2, and VLCDR3 comprise the amino acid sequences SEQ ID NO: 9, SEQ ID NO: 10, and SEQ ID NO: 11 with zero, one, or two amino acid substitutions, respectively; (b) the VHCDR1, VHCDR2, and VHCDR3 comprise the amino acid sequences SEQ ID NO: 130, SEQ ID NO: 132, and SEQ ID NO: 135 with zero, one, or two amino acid substitutions, respectively, and the VLCDR1, VLCDR2, and VLCDR3 comprise the amino acid sequences SEQ ID NO: 138, SEQ ID NO: 140, and SEQ ID NO: 142 with zero, one, or two amino acid substitutions, respectively; (c) the VHCDR1, VHCDR2, and VHCDR3 comprise the amino acid sequences SEQ ID NO: 130, SEQ ID NO: 132, and SEQ ID NO: 135 with zero, one, or two amino acid substitutions, respectively, and the VLCDR1, VLCDR2, and VLCDR3 comprise the amino acid sequences SEQ ID NO: 138, SEQ ID NO: 140, and SEQ ID NO: 143 with zero, one, or two amino acid substitutions, respectively; (d) the VHCDR1, VHCDR2, and VHCDR3 comprise the amino acid sequences SEQ ID NO: 131, SEQ ID NO: 133, and SEQ ID NO: 136 with zero, one, or two amino acid substitutions, respectively, and the VLCDR1, VLCDR2, and VLCDR3 comprise the amino acid sequences SEQ ID NO: 139, SEQ ID NO: 141, and SEQ ID NO: 144 with zero, one, or two amino acid substitutions, respectively; (e) the VHCDR1, VHCDR2, and VHCDR3 comprise the amino acid sequences SEQ ID NO: 131, SEQ ID NO: 134, and SEQ ID NO: 136 with zero, one, or two amino acid substitutions, respectively, and the VLCDR1, VLCDR2, and VLCDR3 comprise the amino acid sequences SEQ ID NO: 139, SEQ ID NO: 141, and SEQ ID NO: 145 with zero, one, or two amino acid substitutions, respectively; or (f) the VHCDR1, VHCDR2, and VHCDR3 comprise the amino acid sequences SEQ ID NO: 131, SEQ ID NO: 134, and SEQ ID NO: 137 with zero, one, or two amino acid substitutions, respectively, and the VLCDR1, VLCDR2, and VLCDR3 comprise the amino acid sequences SEQ ID NO: 139, SEQ ID NO: 141, and SEQ ID NO: 146 with zero, one, or two amino acid substitutions, respectively.

In certain embodiments, the scFv fragment includes a heavy chain variable region (VH) and a light chain variable region (VL), where the VH includes the VH complementarity-determining regions VHCDR1, VHCDR2, and VHCDR3 with the amino acid sequences SEQ ID NO: 5, SEQ ID NO: 6, and SEQ ID NO: 7, respectively, or SEQ ID NO: 5, SEQ ID NO: 6, or SEQ ID NO: 7 with one, two, or three amino acid substitutions in one or more of the VHCDRs, and where the VL includes the VL complementarity-determining regions VLCDR1, VLCDR2, and VLCDR3 with the amino acid sequences SEQ ID NO: 9, SEQ ID NO: 10, and SEQ ID NO: 11, respectively, or SEQ ID NO: 9, SEQ ID NO: 10, and SEQ ID NO: 11 with one, two, or three amino acid substitutions in one or more of the VLCDRs. In certain embodiments, the scFv fragment comprises the VH and VL amino acid sequences SEQ ID NO: 4 and SEQ ID NO: 8, SEQ ID NO: 119 and SEQ ID NO: 120, SEQ ID NO: 121 and SEQ ID NO: 122, SEQ ID NO: 123 and SEQ ID NO: 124, SEQ ID NO: 125 and SEQ ID NO: 126, or SEQ ID NO: 127 and SEQ ID NO: 128, respectively.

In certain embodiments, the scFv fragment includes the VH amino acid sequence SEQ ID NO: 4 and the VL amino acid sequence SEQ ID NO: 8. In other embodiments, the scFv fragment includes a heavy chain variable region (VH) and a light chain variable region (VL), where the VH and VL include the amino acid sequences SEQ ID NO: 13 and SEQ ID NO: 14, respectively. In certain embodiments, the modified J chain includes amino acids 20 to 420 of SEQ ID NO: 12, amino acids 20 to 412 of SEQ ID NO: 15, or amino acids 23 to 415 of SEQ ID NO: 16.

In certain embodiments, the immune effector cell is an NK cell, and where the scFv fragment specifically binds to CD16.

In certain embodiments, the modified J-chain can further include an immune stimulatory agent (“ISA”) fused or chemically conjugated to the J-chain or fragment or variant thereof. In certain embodiments, the ISA includes a cytokine or receptor-binding fragment or variant thereof. In certain embodiments, the ISA includes (a) an interleukin-15 (IL-15) protein or receptor-binding fragment or variant thereof (“I”), and (b) an interleukin-15 receptor-α (IL-15Rα) fragment including the sushi domain or a variant thereof capable of associating with I (“R”), where the J-chain or fragment or variant thereof and at least one of I and R are associated as a fusion protein, and where I and R can associate to function as the ISA. In certain embodiments, the ISA can be fused to the J-chain via a peptide linker.

In certain embodiments, each binding unit of the provided binding molecule further includes two light chains, each including a kappa or lambda light chain constant region and at least a light chain variable region (VL) portion of a binding unit-associated antigen binding domain.

In certain embodiments, the provided binding molecule includes at least four, at least five, at least six, at least seven, at least eight, at least nine, or ten binding unit-associated antigen-binding domains that specifically bind to CD123. In certain embodiments, the at least three, at least four, at least five, at least six, at least seven, at least eight, at least nine, or least ten binding unit-associated antigen-binding domains bind to the same CD123 epitope. In certain embodiments, all the binding unit-associated antigen binding domains of the provided binding molecule are identical.

In certain embodiments, the binding unit-associated antigen-binding domains include a heavy chain variable region (VH) and a light chain variable region (VL), where the VH and VL include six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 include the CDRs of an antibody having VH and VL amino acid sequences including or contained within SEQ ID NO: 32 and SEQ ID NO: 33, SEQ ID NO: 37 and SEQ ID NO: 38, SEQ ID NO: 42 and SEQ ID NO: 43, SEQ ID NO: 44 and SEQ ID NO: 45, SEQ ID NO: 46 and SEQ ID NO: 47, SEQ ID NO: 48 and SEQ ID NO: 49, SEQ ID NO: 50 and SEQ ID NO: 51, SEQ ID NO: 52 and SEQ ID NO: 53, SEQ ID NO: 54 and SEQ ID NO: 55, SEQ ID NO: 56 and SEQ ID NO: 57, SEQ ID NO: 58 and SEQ ID NO: 59, SEQ ID NO: 60 and SEQ ID NO: 61, SEQ ID NO: 62 and SEQ ID NO: 63, SEQ ID NO: 64 and SEQ ID NO: 65, SEQ ID NO: 66 and SEQ ID NO: 67, SEQ ID NO: 68 and SEQ ID NO: 69, SEQ ID NO: 70 and SEQ ID NO: 71, SEQ ID NO: 72 and SEQ ID NO: 73, SEQ ID NO: 74 and SEQ ID NO: 75, SEQ ID NO: 76 and SEQ ID NO: 77, SEQ ID NO: 78 and SEQ ID NO: 79, SEQ ID NO: 80 and SEQ ID NO: 81, SEQ ID NO: 82 and SEQ ID NO: 83, SEQ ID NO: 84 and SEQ ID NO: 85, SEQ ID NO: 86 and SEQ ID NO: 87, SEQ ID NO: 88 and SEQ ID NO: 89, SEQ ID NO: 90 and SEQ ID NO: 91, SEQ ID NO: 92 and SEQ ID NO: 93, SEQ ID NO: 94 and SEQ ID NO: 95, SEQ ID NO: 96 and SEQ ID NO: 97, SEQ ID NO: 98 and SEQ ID NO: 99, SEQ ID NO: 100 and SEQ ID NO: 101, SEQ ID NO: 102 and SEQ ID NO: 103, SEQ ID NO: 107 and SEQ ID NO: 108, SEQ ID NO: 109 and SEQ ID NO: 110, SEQ ID NO: 111 and SEQ ID NO: 112, SEQ ID NO: 113 and SEQ ID NO: 114, SEQ ID NO: 115 and SEQ ID NO: 116, or SEQ ID NO: 117 and SEQ ID NO: 118, respectively or the CDRs of an antibody including the VH and VL amino acid sequences including or contained within SEQ ID NO: 32 and SEQ ID NO: 33, SEQ ID NO: 37 and SEQ ID NO: 38, SEQ ID NO: 42 and SEQ ID NO: 43, SEQ ID NO: 44 and SEQ ID NO: 45, SEQ ID NO: 46 and SEQ ID NO: 47, SEQ ID NO: 48 and SEQ ID NO: 49, SEQ ID NO: 50 and SEQ ID NO: 51, SEQ ID NO: 52 and SEQ ID NO: 53, SEQ ID NO: 54 and SEQ ID NO: 55, SEQ ID NO: 56 and SEQ ID NO: 57, SEQ ID NO: 58 and SEQ ID NO: 59, SEQ ID NO: 60 and SEQ ID NO: 61, SEQ ID NO: 62 and SEQ ID NO: 63, SEQ ID NO: 64 and SEQ ID NO: 65, SEQ ID NO: 66 and SEQ ID NO: 67, SEQ ID NO: 68 and SEQ ID NO: 69, SEQ ID NO: 70 and SEQ ID NO: 71, SEQ ID NO: 72 and SEQ ID NO: 73, SEQ ID NO: 74 and SEQ ID NO: 75, SEQ ID NO: 76 and SEQ ID NO: 77, SEQ ID NO: 78 and SEQ ID NO: 79, SEQ ID NO: 80 and SEQ ID NO: 81, SEQ ID NO: 82 and SEQ ID NO: 83, SEQ ID NO: 84 and SEQ ID NO: 85, SEQ ID NO: 86 and SEQ ID NO: 87, SEQ ID NO: 88 and SEQ ID NO: 89, SEQ ID NO: 90 and SEQ ID NO: 91, SEQ ID NO: 92 and SEQ ID NO: 93, SEQ ID NO: 94 and SEQ ID NO: 95, SEQ ID NO: 96 and SEQ ID NO: 97, SEQ ID NO: 98 and SEQ ID NO: 99, SEQ ID NO: 100 and SEQ ID NO: 101, SEQ ID NO: 102 and SEQ ID NO: 103, SEQ ID NO: 107 and SEQ ID NO: 108, SEQ ID NO: 109 and SEQ ID NO: 110, SEQ ID NO: 111 and SEQ ID NO: 112, SEQ ID NO: 113 and SEQ ID NO: 114, SEQ ID NO: 115 and SEQ ID NO: 116, or SEQ ID NO: 117 and SEQ ID NO: 118, respectively, except for one or two amino acid substitutions in one or more of the CDRs.

In certain embodiments, the binding unit-associated antigen-binding domains include an antibody VH and a VL, where the VH and VL include amino acid sequences at least 80%, at least 85%, at least 90%, at least 95% or 100% identical to the mature VH and VL amino acid sequences including or contained within SEQ ID NO: 32 and SEQ ID NO: 33, SEQ ID NO: 37 and SEQ ID NO: 38, SEQ ID NO: 42 and SEQ ID NO: 43, SEQ ID NO: 44 and SEQ ID NO: 45, SEQ ID NO: 46 and SEQ ID NO: 47, SEQ ID NO: 48 and SEQ ID NO: 49, SEQ ID NO: 50 and SEQ ID NO: 51, SEQ ID NO: 52 and SEQ ID NO: 53, SEQ ID NO: 54 and SEQ ID NO: 55, SEQ ID NO: 56 and SEQ ID NO: 57, SEQ ID NO: 58 and SEQ ID NO: 59, SEQ ID NO: 60 and SEQ ID NO: 61, SEQ ID NO: 62 and SEQ ID NO: 63, SEQ ID NO: 64 and SEQ ID NO: 65, SEQ ID NO: 66 and SEQ ID NO: 67, SEQ ID NO: 68 and SEQ ID NO: 69, SEQ ID NO: 70 and SEQ ID NO: 71, SEQ ID NO: 72 and SEQ ID NO: 73, SEQ ID NO: 74 and SEQ ID NO: 75, SEQ ID NO: 76 and SEQ ID NO: 77, SEQ ID NO: 78 and SEQ ID NO: 79, SEQ ID NO: 80 and SEQ ID NO: 81, SEQ ID NO: 82 and SEQ ID NO: 83, SEQ ID NO: 84 and SEQ ID NO: 85, SEQ ID NO: 86 and SEQ ID NO: 87, SEQ ID NO: 88 and SEQ ID NO: 89, SEQ ID NO: 90 and SEQ ID NO: 91, SEQ ID NO: 92 and SEQ ID NO: 93, SEQ ID NO: 94 and SEQ ID NO: 95, SEQ ID NO: 96 and SEQ ID NO: 97, SEQ ID NO: 98 and SEQ ID NO: 99, SEQ ID NO: 100 and SEQ ID NO: 101, SEQ ID NO: 102 and SEQ ID NO: 103, SEQ ID NO: 107 and SEQ ID NO: 108, SEQ ID NO: 109 and SEQ ID NO: 110, SEQ ID NO: 111 and SEQ ID NO: 112, SEQ ID NO: 113 and SEQ ID NO: 114, SEQ ID NO: 115 and SEQ ID NO: 116, or SEQ ID NO: 117 and SEQ ID NO: 118, respectively.

In certain embodiments, the provided binding molecule is a dimeric binding molecule that includes two bivalent binding units, where each binding unit includes two antibody heavy chains, each including an IgA or IgA-like heavy chain constant region or multimerizing fragment thereof. In certain embodiments the provided dimeric binding molecule further includes a secretory component, or fragment or variant thereof. In certain embodiments, the IgA or IgA-like heavy chain constant regions or multimerizing fragments thereof each include a Cα3 and a tailpiece (tp) domain, and can further include a Cα1 domain, a Cα2 domain, an IgA hinge region, or any combination thereof. In certain embodiments, the IgA or IgA-like heavy chain constant regions are human IgA or IgA-like constant regions and can include the amino acid sequence SEQ ID NO: 24, SEQ ID NO: 25, or any multimerizing variant or fragment thereof. In certain embodiments each binding unit includes two IgA or IgA-like heavy chains each including a VH situated amino terminal to the IgA constant region or fragment thereof, and two immunoglobulin light chains each including a VL situated amino terminal to an immunoglobulin light chain constant region.

In certain embodiments, the provided binding molecule is a pentameric binding molecule including five bivalent binding units, where each binding unit includes two IgM or IgM-like heavy chain constant regions or multimerizing fragments thereof. In certain embodiments, the IgM or IgM-like heavy chain constant regions or multimerizing fragments thereof each include a Cμ4 domain and a tailpiece (tp) domain or fragment or variant thereof and can further include a Cμ1 domain, a Cμ2 domain, a Cμ3 domain, or any combination thereof. In certain embodiments the IgM or IgM-like heavy chain constant regions are human IgM constant regions and can include the amino acid sequence SEQ ID NO: 22, SEQ ID NO: 23, or a multimerizing variant or fragment thereof. In certain embodiments each binding unit includes two IgM heavy chains each including a VH situated amino terminal to the IgM constant region or fragment thereof, and two immunoglobulin light chains each including a VL situated amino terminal to an immunoglobulin light chain constant region.

In certain embodiments, the binding units include variant human IgM constant regions, where the multimeric binding molecule has reduced CDC activity relative to a multimeric binding molecule including IgM heavy chain constant regions including the amino acid sequence SEQ ID NO: 22, SEQ ID NO: 23, or a multimerizing variant or fragment thereof. In certain embodiments, each IgM heavy chain constant region includes a variant of the amino acid sequence SEQ ID NO: 22 or SEQ ID NO: 23, where the variant includes an amino acid substitution at position P311 of SEQ ID NO: 22 or SEQ ID NO: 23, an amino acid substitution at position P313 of SEQ ID NO: 22 or SEQ ID NO: 23, or amino acid substitutions at positions P311 and P313 of SEQ ID NO: 22 or SEQ ID NO: 23.

In certain embodiments, the binding units include variant human IgM constant regions with one or more single amino acid substitutions, deletions, or insertions relative to a reference IgM heavy chain constant region identical to the variant IgM heavy chain constant regions except for the one or more single amino acid substitutions, deletions, or insertions; where the binding molecule exhibits increased serum half-life upon administration to a subject animal relative to a multimeric binding molecule including the reference IgM heavy chain constant regions, and is administered in the same way to the same animal species. In certain embodiments, the variant IgM heavy chain constant regions include amino acid substitutions at one or more amino acid positions corresponding to amino acid, E345A, S401A, E402A, or E403A of the wild-type human IgM constant region SEQ ID NO: 22 or SEQ ID NO: 23.

This disclosure further provides composition, e.g., a pharmaceutical composition, that includes the provided binding molecule.

This disclosure also provides a polynucleotide that includes a nucleic acid sequence that encodes a polypeptide subunit of the provided binding molecule.

In certain embodiments, the polypeptide subunit includes an IgM or IgM-like heavy chain constant region and at least an antibody VH portion of the binding unit-associated antigen-binding domain of the binding molecule. In certain embodiments, the polypeptide subunit includes a human IgM constant region or fragment thereof fused to the C-terminal end of a VH that includes: (a) HCDR1, HCDR2, and HCDR3 regions including the CDRs contained in the VH amino acid sequence including or contained within SEQ ID NO: 32, SEQ ID NO: 37, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 107, SEQ ID NO: 109, SEQ ID NO: 111, SEQ ID NO: 113, SEQ ID NO: 115, or SEQ ID NO: 117; or the CDRs contained in the VH amino acid sequence including or contained within SEQ ID NO: 32, SEQ ID NO: 37, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 107, SEQ ID NO: 109, SEQ ID NO: 111, SEQ ID NO: 113, SEQ ID NO: 115, or SEQ ID NO: 117 except for one or two single amino acid substitutions in one or more of the HCDRs; or (b) an amino acid sequence at least 80%, at least 85%, at least 90%, at least 95% or 100% identical to the mature VH amino acid sequence including or contained within SEQ ID NO: 32, SEQ ID NO: 37, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 107, SEQ ID NO: 109, SEQ ID NO: 111, SEQ ID NO: 113, SEQ ID NO: 115, or SEQ ID NO: 117.

In certain embodiments, the polypeptide subunit includes a light chain constant region and an antibody VL portion of the antigen-binding domain of the multimeric binding molecule. In certain embodiments, the polypeptide subunit includes a human kappa or lambda light chain constant region or fragment thereof fused to the C-terminal end of a VL including: (a) LCDR1, LCDR2, and LCDR3 regions including the CDRs contained in the VL amino acid sequence including or contained within SEQ ID NO: 33, SEQ ID NO: 38, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 108, SEQ ID NO: 110, SEQ ID NO: 112, SEQ ID NO: 114, SEQ ID NO: 116, or SEQ ID NO: 118; or the CDRs contained in the VL amino acid sequence SEQ ID NO: 33, SEQ ID NO: 38, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 108, SEQ ID NO: 110, SEQ ID NO: 112, SEQ ID NO: 114, SEQ ID NO: 116, or SEQ ID NO: 118 except for one or two single amino acid substitutions in one or more of the LCDRs; or (b) an amino acid sequence at least 80%, at least 85%, at least 90%, at least 95% or 100% identical to the mature VL amino acid sequence including or contained within SEQ ID NO: 33, SEQ ID NO: 38, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 108, SEQ ID NO: 110, SEQ ID NO: 112, SEQ ID NO: 114, SEQ ID NO: 116, or SEQ ID NO: 118.

In certain embodiments, the polypeptide subunit includes a modified J-chain, where the modified J-chain includes a wild-type J-chain or a functional fragment or variant thereof and a J-chain-associated antigen-binding domain that specifically binds to an immune effector cell. In certain embodiments, the modified J-chain includes an amino acid sequence at least 80%, 85%, 90%, 95%, or 100% identical to amino acids 20 to 420 of SEQ ID NO: 12, amino acids 20 to 412 of SEQ ID NO: 15, or amino acids 23 to 415 of SEQ ID NO: 16.

This disclosure further provides a composition that includes two or more of the aforementioned polynucleotides. The polynucleotides can be situated are on two or more separate vectors, or on a single vector. Such a vector or vectors are also provided.

This disclosure also provides a host cell that includes one or more provided polynucleotide(s) or the provided vector or vectors, where the host cell can express the provided binding molecule or a subunit thereof. This disclosure further provides a method of producing the provided binding molecule, where the method includes culturing the host cell and then recovering the binding molecule.

This disclosure also provides a method of treating cancer or other malignancy, where the method includes administering to a subject in need of cancer treatment an effective amount of the provided binding molecule, where the binding molecule can induce immune effector cell-mediated killing of cancer cells. In certain embodiments the cancer or malignancy is a hematologic cancer or malignancy, for example, acute myeloid leukemia (AML), myelodysplastic syndrome (MDS), chronic myeloid leukemia (CML), B-cell acute lymphoblastic leukemia (B-cell ALL), classical Hodgkin's lymphoma, hairy cell leukemia, chronic lymphocytic leukemia (CLL), systemic mastocytosis, or plasmacytoid dendritic cell leukemia. In certain embodiments, the J-chain-associated antigen-binding domain binds to CD3s, and the binding molecule induces T-cell mediated killing of malignant cells. In certain embodiments the treatment results in reduced cytokine release relative to a corresponding IgG-based anti-CD123 anti-CD3 bispecific antibody. In certain embodiments, the subject to be treated is a human subject.

### BRIEF DESCRIPTION OF THE DRAWINGS/FIGURES

FIGS. 1A-1D show expression, proper assembly, and purification by size exclusion chromatography (SEC) of anti-CD123×CD3 IgM #1 (heavy chain: amino acids 20 to 592 of SEQ ID NO: 35, light chain: amino acids 21 to 240 of SEQ ID NO: 36, modified J-chain, amino acids 20 to 420 of SEQ ID NO: 12) and anti-CD123×CD3 IgM #2 (heavy chain: amino acids 20 to 589 of SEQ ID NO: 40, light chain amino acids 21 to 234 of SEQ ID NO: 41, modified J-chain, amino acids 20 to 420 of SEQ ID NO: 12). FIG. 1A: non reduced gel; FIG. 1B: reduced gel; FIG. C: size exclusion chromatograph trace to show purification of anti-CD123×CD3 IgM #1; FIG. 1D: size exclusion chromatograph trace to show purification of anti-CD123×CD3 IgM #2.

FIGS. 2A-2B show expression, proper assembly, and purification by size exclusion chromatography (SEC) of anti-CD123×CD3 IgG #1 (first heavy chain: SEQ ID NO: 104, light chain a SEQ ID NO: 105, second heavy chain: SEQ ID NO: 6).

FIG. 3 shows that anti-CD123×CD3 IgM #1 (triangles) and anti-CD123×CD3 IgM #2 (inverted triangles) bind to CD123 in an ELISA assay. Also shown are CD123 binding of mono-specific IgG versions anti-CD123 IgG #1 (asterisk, heavy chain: amino acids 20 to 469 of SEQ ID NO: 34, light chain, amino acids 21 to 240 of SEQ ID NO: 36), and anti-CD123 IgG #2 (star, heavy chain: amino acids 20 to 464 of SEQ ID NO: 39, light chain, amino acids 21 to 234 of SEQ ID NO: 41).

FIG. 4A shows that anti-CD123×CD3 IgM #1 (triangles) and anti-CD123×CD3 IgM #2 (inverted triangles) bind to CD3ε in an ELISA assay, but that the mono-specific IgG anti-CD123 constructs do not. FIG. 4B compares binding of anti-CD123×CD3 IgM #1 (triangles) and anti CD123×CD3 IgG #1 (open circles) to CD3ε in an ELISA assay.

FIG. 5A-5D shows binding of the IgM and IgG bispecific antibodies to CD123 at different protein concentrations measured by ELISA. FIG. 5A: 3 μg/ml CD123, FIG. 5B: 1 μg/ml CD123; FIG. 5C: 0.33 μg/ml CD123; and FIG. 5D: 0.11 μg/ml CD123.

FIG. 6 shows quantification of CD123 expressed on the surface of various AML cell lines.

FIG. 7 shows binding of Anti-CD123×CD3 IgM #1 to three different AML cell lines, Kg-1a, MOLM-13, and MV4-11, and a Burkitt's lymphoma cell line Namalwa (CD123 negative) via flow cytometry. Top row: control anti-CD123 antibody 7G3; bottom row: anti-CD123×CD3 IgM #1, anti-CD123×CD3 IgM #3 and anti-CD123×CD3 IgM #4.

FIGS. 8A-8C show T cell dependent killing of CD123-expressing AML cell lines THP-1 (FIG. 8A) and MV4-11 (FIG. 8B) in the presence of Anti-CD123×CD3 IgM #1, where Namalwa cells, which do not express CD123, were not killed (FIG. 5C).

FIG. 9 shows that anti-CD123×CD3 IgM #1 enhances the CD25 activation marker on CD8+ T cells but not on CD4+ T cells in a TDCC assay on MV4-11 cells.

FIGS. 10A and 10B compare anti-CD123×CD3 IgM #1 (triangles) and anti-CD123×CD3 IgG #1 (open circles) in a pan-TDCC assay on MV4-11 cells (panel A) and THP-1 cells (panel B) after 96 hours. Open circles: anti-CD123×CD3 IgG #1, closed triangles: anti-CD123×CD3 IgM #1.

FIGS. 11A-11D show a comparison of cytokine release between anti-CD123×CD3 IgG #1 and anti-CD123×CD3 IgM #1 in a TDCC assay on MV4-11 cells. FIG. A: interferon gamma (IFNγ) release; FIG. B: interleukin-6 (IL-6) release; FIG. C: TNFα release; FIG. D: interleukin-10 (IL10) release.

FIGS. 12A-12D show a comparison of cytokine release between anti-CD123×CD3 IgG #1 and anti-CD123×CD3 IgM #1 in a TDCC assay on THP-1 cells. FIG. A: interferon gamma (IFNγ) release; FIG. B: interleukin-6 (IL-6) release; FIG. C: TNFα release; FIG. D: interleukin-10 (IL10) release.

FIG. 13 shows binding of IgM bispecific antibodies to CD123 at different protein concentrations measured by ELISA.

FIG. 14 shows binding of IgM bispecific antibodies to MV4-11 cells that express CD123.

FIGS. 15A-15B show T cell dependent killing of CD123-expressing AML cell lines THP-1 (FIG. 15A) and PL21 (FIG. 15B) in the presence of Anti-CD123×CD3 IgM antibodies with various CD123 binding domains.

FIGS. 16A-16B show T cell dependent killing of CD123-expressing AML cell lines THP-1 (FIG. 16A) and PL21 (FIG. 16B) in the presence of Anti-CD123×CD3 IgM antibodies with various CD3 binding domains.

FIG. 17 shows T cell dependent killing of CD123-expressing AML cell line MV4-11 in the presence of Anti-CD123×CD3 IgM antibodies with various CD3 binding domains and J chains.

FIGS. 18A-18F show the resulting tumor viability (FIGS. 18A, 18D), T cell proliferation (FIGS. 18B, 18E), and T cell activation (FIGS. 18C, 18F) for cells treated with anti-CD123×CD3 IgM or IgG antibodies when the T cells are CD8+ T cells (FIGS. 18A-18C) or CD4+ T cells (FIGS. 18D-18F).

FIGS. 19A-19E show a comparison of cytokine release between anti-CD123×CD3 IgG and anti-CD123×CD3 IgM antibodies in a TDCC assay for the cytokines interferon gamma (IFNγ) (FIG. 19A); tumor necrosis factor alpha (TNFα) (FIG. 19B); interleukin-6 (IL-6) (FIG. 19C); interleukin-10 (IL-10) (FIG. 19D); interleukin-2 (IL-2) (FIG. 19E).

### DETAILED DESCRIPTION

### Definitions

It is to be noted that the term “a” or “an” entity refers to one or more of that entity; for example, “a binding molecule,” is understood to represent one or more binding molecules. As such, the terms “a” (or “an”), “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various embodiments or embodiments of the disclosure, which can be had by reference to the specification as a whole. Accordingly, the terms defined immediately below are more fully defined by reference to the specification in its entirety.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, and derivatization by known protecting/blocking groups, proteolytic cleavage, or modification by non-naturally occurring amino acids. A polypeptide can be derived from a biological source or produced by recombinant technology but is not necessarily translated from a designated nucleic acid sequence. It can be generated in any manner, including by chemical synthesis.

A polypeptide as disclosed herein can be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides can have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations and are referred to as unfolded. As used herein, the term glycoprotein refers to a protein coupled to at least one carbohydrate moiety that is attached to the protein via an oxygen-containing or a nitrogen-containing side chain of an amino acid, e.g., a serine or an asparagine.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are native or recombinant polypeptides which have been separated, fractionated, or partially or substantially purified by any suitable technique.

As used herein, the term “a non-naturally occurring polypeptide” or any grammatical variants thereof, is a conditional definition that explicitly excludes, but only excludes, those forms of the polypeptide that are, or might be, determined or interpreted by a judge or an administrative or judicial body, to be “naturally-occurring.”

Other polypeptides disclosed herein are fragments, derivatives, analogs, or variants of the foregoing polypeptides, and any combination thereof. The terms “fragment,” “variant,” “derivative” and “analog” as disclosed herein include any polypeptides which retain at least some of the properties of the corresponding native antibody or polypeptide, for example, specifically binding to an antigen. Fragments of polypeptides include, for example, proteolytic fragments, as well as deletion fragments, in addition to specific antibody fragments discussed elsewhere herein. Variants of, e.g., a polypeptide include fragments as described above, and polypeptides with altered amino acid sequences due to amino acid substitutions, deletions, or insertions. In certain embodiments, variants can be non-naturally occurring. Non-naturally occurring variants can be produced using art-known mutagenesis techniques. Variant polypeptides can comprise conservative or non-conservative amino acid substitutions, deletions or additions. Derivatives are polypeptides that have been altered to exhibit additional features not found on the original polypeptide. Examples include fusion proteins. Variant polypeptides can also be referred to herein as “polypeptide analogs.” As used herein a “derivative” of a polypeptide can also refer to a subject polypeptide having one or more amino acids chemically derivatized by reaction of a functional side group. Also included as “derivatives” are those peptides that contain one or more derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and omithine can be substituted for lysine.

A “conservative amino acid substitution” is one in which one amino acid is replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the sequences of the polypeptides and antibodies of the present disclosure do not abrogate the binding of the polypeptide or antibody containing the amino acid sequence, to the antigen to which the antibody binds. Methods of identifying nucleotide and amino acid conservative substitutions which do not eliminate antigen-binding are well-known in the art (see, e.g., Brummell et al., Biochem. 32: 1180-1 187 (1993); Kobayashi et al., Protein Eng. 12(10):879-884 (1999); and Burks et al., Proc. Natl. Acad. Sci. USA 94: 412-417 (1997)).

The term “polynucleotide” is intended to encompass a singular nucleic acid as well as plural nucleic acids and refers to an isolated nucleic acid molecule or construct, e.g., messenger RNA (mRNA), cDNA, or plasmid DNA (pDNA). A polynucleotide can comprise a conventional phosphodiester bond or a non-conventional bond (e.g., an amide bond, such as found in peptide nucleic acids (PNA)). The terms “nucleic acid” or “nucleic acid sequence” refer to any one or more nucleic acid segments, e.g., DNA or RNA fragments, present in a polynucleotide.

By an “isolated” nucleic acid or polynucleotide is intended any form of the nucleic acid or polynucleotide that is separated from its native environment. For example, gel-purified polynucleotide, or a recombinant polynucleotide encoding a polypeptide contained in a vector would be considered to be “isolated.” Also, a polynucleotide segment, e.g., a PCR product, which has been engineered to have restriction sites for cloning is considered to be “isolated.” Further examples of an isolated polynucleotide include recombinant polynucleotides maintained in heterologous host cells or purified (partially or substantially) polynucleotides in a non-native solution such as a buffer or saline. Isolated RNA molecules include in vivo or in vitro RNA transcripts of polynucleotides, where the transcript is not one that would be found in nature. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, the term “a non-naturally occurring polynucleotide” or any grammatical variants thereof, is a conditional definition that explicitly excludes, but only excludes, those forms of the nucleic acid or polynucleotide that are, or might be, determined or interpreted by a judge, or an administrative or judicial body, to be “naturally-occurring.”

As used herein, a “coding region” is a portion of nucleic acid which consists of codons translated into amino acids. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector can contain a single coding region, or can comprise two or more coding regions, e.g., a single vector can separately encode an immunoglobulin heavy chain variable region and an immunoglobulin light chain variable region. In addition, a vector, polynucleotide, or nucleic acid can include heterologous coding regions, either fused or unfused to another coding region. Heterologous coding regions include without limitation, those encoding specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally can include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA in predetermined cells. Other transcription control elements, besides a promoter, for example enhancers, operators, repressors, and transcription termination signals, can be operably associated with the polynucleotide to direct cell-specific transcription.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit ß-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picomaviruses (particularly an internal ribosome entry site, or IRES, also referred to as a CITE sequence).

In other embodiments, a polynucleotide can be RNA, for example, in the form of messenger RNA (mRNA), transfer RNA, or ribosomal RNA.

Polynucleotide and nucleic acid coding regions can be associated with additional coding regions which encode secretory or signal peptides, which direct the secretion of a polypeptide encoded by a polynucleotide as disclosed herein. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells can have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse ß-glucuronidase.

As used herein, the term “binding molecule” refers in its broadest sense to a molecule that specifically binds to a binding target, e.g., an epitope or an antigenic determinant. As described further herein, a binding molecule can comprise one of more “antigen-binding domains” described herein. A non-limiting example of a binding molecule is an antibody or antibody-like molecule as described in detail herein that retains antigen-specific binding. In certain embodiments a “binding molecule” comprises an antibody or antibody-like molecule as described in detail herein.

As used herein, the terms “binding domain” or “antigen-binding domain” (can be used interchangeably) refer to a region of a binding molecule, e.g., an antibody or antibody-like molecule, that is necessary and sufficient to specifically bind to a binding target, e.g., an epitope. For example, an “Fv,” e.g., a heavy chain variable region and a light chain variable region of an antibody, either as two separate polypeptide subunits or as a single chain, is considered to be a “binding domain.” Other antigen-binding domains include, without limitation, the heavy chain variable region (VHH) of an antibody derived from a camelid species, or six immunoglobulin complementarity determining regions (CDRs) expressed in a scaffold, e.g., a fibronectin scaffold. A “binding molecule,” or “antibody” as described herein can include one, two, three, four, five, six, seven, eight, nine, ten, eleven, twelve, or even more “antigen-binding domains.” As used herein, a “binding unit-associated antigen-binding domain” refers to an antigen binding domain that is part of an antibody heavy chain and/or an antibody light chain. The term “J-chain-associated antigen-binding domain” refers to an antigen binding domain that is associated with a modified J-chain as described herein, for example, a scFv fused to a wild type human J-chain, or functional fragment or variant thereof.

The terms “antibody” and “immunoglobulin” can be used interchangeably herein. An antibody (or a fragment, variant, or derivative thereof as disclosed herein) includes at least the variable domain of a heavy chain (for camelid species) or at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988). Unless otherwise stated, the term “antibody” encompasses anything ranging from a small antigen-binding fragment of an antibody to a full sized antibody, e.g., an IgG antibody that includes two complete heavy chains and two complete light chains, an IgA antibody that includes four complete heavy chains and four complete light chains and optionally includes a J-chain and/or a secretory component, or an IgM antibody that includes ten or twelve complete heavy chains and ten or twelve complete light chains and optionally includes a J-chain or functional fragment thereof.

The term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4 or α1-α2). It is the nature of this chain that determines the “isotype” of the antibody as IgG, IgM, IgA, IgD, or IgE, respectively. The immunoglobulin subclasses (subtypes) e.g., IgG₁, IgG₂, IgG₃, IgG₄, IgA₁, IgA₂, etc. are well characterized and are known to confer functional specialization. Modified versions of each of these immunoglobulins are readily discernible to the skilled artisan in view of the instant disclosure and, accordingly, are within the scope of this disclosure.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are expressed, e.g., by hybridomas, B cells or genetically engineered host cells. In the heavy chain, the amino acid sequences run from an N-terminus at the forked ends of the Y configuration to the C-terminus at the bottom of each chain. The basic structure of certain antibodies, e.g., IgG antibodies, includes two heavy chain subunits and two light chain subunits covalently connected via disulfide bonds to form a “Y” structure, also referred to herein as an “H2L2” structure, or a “binding unit.”

The term “binding unit” is used herein to refer to the portion of a binding molecule, e.g., an antibody, antibody-like molecule, antigen-binding fragment thereof, or multimerizing fragment thereof, which corresponds to a standard “H2L2” immunoglobulin structure, e.g., two heavy chains or fragments thereof and two light chains or fragments thereof. In certain embodiments a binding unit can correspond to two heavy chains, e.g., in a camelid antibody. In certain embodiments, e.g., where the binding molecule is a bivalent IgG antibody or antigen-binding fragment thereof, the terms “binding molecule” and “binding unit” are equivalent. In other embodiments, e.g., where the binding molecule is multimeric, e.g., a dimeric IgA antibody or IgA-like antibody, a pentameric IgM antibody or IgM-like antibody, or a hexameric IgM antibody or IgM-like antibody, the binding molecule comprises two or more “binding units.” Two in the case of an IgA dimer, or five or six in the case of an IgM pentamer or hexamer, respectively. A binding unit need not include full-length antibody heavy and light chains, but will typically be bivalent, i.e., will include two “antigen-binding domains,” as defined above. As used herein, certain binding molecules provided in this disclosure are “dimeric,” and include two bivalent binding units that include IgA constant regions or multimerizing fragments thereof. Certain binding molecules provided in this disclosure are “pentameric” or “hexameric,” and include five or six bivalent binding units that include IgM constant regions or multimerizing fragments thereof. A binding molecule, e.g., an antibody or antibody-like molecule, comprising two or more, e.g., two, five, or six binding units, is referred to herein as “multimeric.”

The term “J-chain” as used herein refers to the J-chain of native sequence IgM or IgA antibodies of any animal species, any functional fragment thereof, derivative thereof, and/or variant thereof, including the mature human J-chain, the amino acid sequence of which is presented as SEQ ID NO: 2. Various J-chain variants and modified J-chain derivatives are disclosed herein. As persons of ordinary skill in the art will recognize, “a functional fragment” or a “functional variant” includes those fragments and variants that can associate with IgM heavy chain constant regions to form a pentameric IgM antibody (or alternatively can associate with IgA heavy chain constant regions to form a dimeric IgA antibody).

The term “modified J-chain” is used herein to refer to a derivative of a native sequence J-chain polypeptide comprising a heterologous moiety, e.g., a heterologous polypeptide, e.g., an extraneous binding domain introduced into the native sequence. The introduction can be achieved by any means, including direct or indirect fusion of the heterologous polypeptide or other moiety or by attachment through a peptide or chemical linker. The term “modified human J-chain” encompasses, without limitation, a native sequence human J-chain comprising the amino acid sequence of SEQ ID NO: 2 or functional fragment thereof, or functional variant thereof, modified by the introduction of a heterologous moiety, e.g., a heterologous polypeptide, e.g., an extraneous binding domain. In certain embodiments the heterologous moiety does not interfere with efficient polymerization of IgM into a pentamer and binding of such polymers to a target. Exemplary modified J-chains can be found, e.g., in U.S. Pat. Nos. 9,951,134 and 10,618,978, in U.S. Patent Application Publication No. US-2019-0185570, each of which is incorporated herein by reference in its entirety.

As used herein, the terms “IgM-derived binding molecule,” “IgM-like antibody,” “IgM-like binding unit,” or “IgM-like heavy chain constant region” refer to a variant antibody-derived binding molecule, antibody, binding unit, or heavy chain constant region that still retains the structural portions of an IgM heavy chain necessary to confer the ability to form multimers, i.e., hexamers, or in association with J-chain, form pentamers. An IgM-like antibody or IgM-derived binding molecule typically includes at least the Cμ4 and tailpiece (tp) domains of the IgM constant region but can include heavy chain constant region domains from other antibody isotypes, e.g., IgG, from the same species or from a different species. An IgM-like antibody or IgM-derived binding molecule can likewise be an antibody fragment in which one or more constant regions are deleted, as long as the IgM-like antibody is capable of forming hexamers and/or pentamers. Thus, an IgM-like antibody or IgM-derived binding molecule can be, e.g., a hybrid IgM/IgG antibody or can be a “multimerizing fragment” of an IgM antibody.

As used herein, the terms “IgA-derived binding molecule,” “IgA-like antibody,” “IgA-like binding unit,” or “IgA-like heavy chain constant region” refer to a variant antibody-derived binding molecule, antibody, binding unit, or heavy chain constant region that still retains the structural portions of an IgA heavy chain necessary to confer the ability to form multimers, i.e., dimers, in association with J-chain. An IgA-like antibody or IgA-derived binding molecule typically includes at least the Cα3 and tailpiece (tp) domains of the IgA constant region but can include heavy chain constant region domains from other antibody isotypes, e.g., IgG, from the same species or from a different species. An IgA-like antibody or IgA-derived binding molecule can likewise be an antibody fragment in which one or more constant regions are deleted, as long as the IgA-like antibody is capable of forming dimers in association with a J-chain. Thus, an IgA-like antibody or IgA-derived binding molecule can be, e.g., a hybrid IgA/IgG antibody or can be a “multimerizing fragment” of an IgA antibody.

The terms “valency,” “bivalent,” “multivalent” and grammatical equivalents, refer to the number of antigen-binding domains in given binding molecule, e.g., antibody or antibody-like molecule, or in a given binding unit. As such, the terms “bivalent”, “tetravalent”, and “hexavalent” in reference to a given binding molecule, e.g., an IgM antibody, IgM-like antibody or multimerizing fragment thereof, denote the presence of two antigen-binding domains, four antigen-binding domains, and six antigen-binding domains, respectively. A typical IgM antibody or IgM-like antibody or IgM-derived binding molecule where each binding unit is bivalent, can have 10 or 12 valencies. A bivalent or multivalent binding molecule, e.g., antibody or antibody-like molecule, can be monospecific, i.e., all of the antigen-binding domains are the same, or can be bispecific or multispecific, e.g., where two or more antigen-binding domains are different, e.g., bind to different epitopes on the same antigen, or bind to entirely different antigens.

The term “epitope” includes any molecular determinant capable of specific binding to an antigen-binding domain of an antibody or antibody-like molecule. In certain embodiments, an epitope can include chemically active surface groupings of molecules such as amino acids, sugar side chains, phosphoryl, or sulfonyl, and, in certain embodiments, can have three-dimensional structural characteristics, and or specific charge characteristics. An epitope is a region of a target that is bound by an antigen-binding domain of an antibody.

The term “target” is used in the broadest sense to include substances that can be bound by a binding molecule, e.g., antibody or antibody-like molecule. A target can be, e.g., a polypeptide, a nucleic acid, a carbohydrate, a lipid, or other molecule. Moreover, a “target” can, for example, be a cell, an organ, or an organism that comprises an epitope that can be bound by a binding molecule, e.g., antibody or antibody-like molecule.

Both the light and heavy chains are divided into regions of structural and functional homology. The terms “constant” and “variable” are used functionally. The variable regions of both the light (VL) and heavy (VH) chains determine antigen recognition and specificity. Conversely, the constant domains of the light chain (CL) and the heavy chain (e.g., CH1, CH2, CH3, or CH4) confer biological properties such as secretion, transplacental mobility, Fc receptor binding, complement binding, and the like. By convention the numbering of the constant region domains increases as they become more distal from the antigen-binding site or amino-terminus of the antibody. The N-terminal portion is a variable region and at the C-terminal portion is a constant region; the CH3 (or CH4 in the case of IgM) and CL domains actually comprise the carboxy-terminus of the heavy and light chain, respectively.

A “full length IgM antibody heavy chain” is a polypeptide that includes, in N-terminal to C-terminal direction, an antibody heavy chain variable domain (VH), an antibody heavy chain constant domain 1 (CM1 or Cμ1), an antibody heavy chain constant domain 2 (CM2 or Cμ2), an antibody heavy chain constant domain 3 (CM3 or Cμ3), and an antibody heavy chain constant domain 4 (CM4 or Cμ4) that can include a tailpiece.

As indicated above, variable region(s) allows a binding molecule, e.g., antibody or antibody-like molecule, to selectively recognize and specifically bind epitopes on antigens. That is, the VL domain and VH domain, or subset of the complementarity determining regions (CDRs), of a binding molecule, e.g., an antibody or antibody-like molecule, combine to form the antigen-binding domain. More specifically, an antigen-binding domain can be defined by three CDRs on each of the VH and VL chains. Certain antibodies form larger structures. For example, IgA can form a molecule that includes two H2L2 binding units and a J-chain covalently connected via disulfide bonds, which can be further associated with a secretory component, and IgM can form a pentameric or hexameric molecule that includes five or six H2L2 binding units and optionally a J-chain covalently connected via disulfide bonds.

The six “complementarity determining regions” or “CDRs” present in an antibody antigen-binding domain are short, non-contiguous sequences of amino acids that are specifically positioned to form the antigen-binding domain as the antibody assumes its three-dimensional configuration in an aqueous environment. The remainder of the amino acids in the antigen-binding domain, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the target antigen. This complementary surface promotes the non-covalent binding of the antibody to its cognate epitope. The amino acids that make up the CDRs and the framework regions, respectively, can be readily identified for any given heavy or light chain variable region by one of ordinary skill in the art, since they have been defined in various different ways (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. These particular regions have been described, for example, by Kabat et al., U.S. Dept. of Health and Human Services, “Sequences of Proteins of Immunological Interest” (1983) and by Chothia et al., J. Mol. Biol. 196:901-917 (1987), which are incorporated herein by reference. The Kabat and Chothia definitions include overlapping or subsets of amino acids when compared against each other. Nevertheless, application of either definition (or other definitions known to those of ordinary skill in the art) to refer to a CDR of an antibody or variant thereof is intended to be within the scope of the term as defined and used herein, unless otherwise indicated. The appropriate amino acids which encompass the CDRs as defined by each of the above cited references are set forth below in Table 1 as a comparison. The exact amino acid numbers which encompass a particular CDR will vary depending on the sequence and size of the CDR. Those skilled in the art can routinely determine which amino acids comprise a particular CDR given the variable region amino acid sequence of the antibody.

TABLE 1
CDR Definitions*

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

*Numbering of all CDR definitions in Table 1 is according to the numbering conventions set forth by Kabat et al. (see below).

Antibody variable domains can also be analyzed, e.g., using the IMGT information system (imgt_dot_cines_dot_fr/) (IMGT®/V-Quest) to identify variable region segments, including CDRs. (See, e.g., Brochet et al., Nucl. Acids Res., 36:W503-508, 2008).

Kabat et al. also defined a numbering system for variable region and constant region sequences that is applicable to any antibody. One of ordinary skill in the art can unambiguously assign this system of “Kabat numbering” to any variable region sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless use of the Kabat numbering system is explicitly noted, however, consecutive numbering is used for all amino acid sequences in this disclosure.

The Kabat numbering system for the human IgM constant domain can be found in Kabat, et al. “Tabulation and Analysis of Amino acid and nucleic acid Sequences of Precursors, V-Regions, C-Regions, J-Chain, T-Cell Receptors for Antigen, T-Cell Surface Antigens, β-2 Microglobulins, Major Histocompatibility Antigens, Thy-1, Complement, C-Reactive Protein, Thymopoietin, Integrins, Post-gamma Globulin, α-2 Macroglobulins, and Other Related Proteins,” U.S. Dept. of Health and Human Services (1991). IgM constant regions can be numbered sequentially (i.e., amino acid #1 starting with the first amino acid of the constant region, or by using the Kabat numbering scheme. A comparison of the numbering of two alleles of the human IgM constant region sequentially (presented herein as SEQ ID NO: 22 (allele IGHM*03) and SEQ ID NO: 23 (allele IGHM*04)) and by the Kabat system is set out below. The underlined amino acid residues are not accounted for in the Kabat system (“X” double underlined below, can be serine (S) (SEQ ID NO: 22) or glycine (G) (SEQ ID NO: 23)):

Sequential (SEQ ID NO: 22 or SEQ ID NO: 23)/KABAT numbering key for IgM heavy chain

| | |
|---|---|
| 1/127 | GSASAPTLFP LVSCENSPSD TSSVAVGCLA QDFLPDSITF SWKYKNNSDI |
| 51/176 | SSTRGFPSVL RGGKYAATSQ VLLPSKDVMQ GTDEHVVCKV QHPNGNKEKN |
| 101/226 | VPLPVIAELP PKVSVFVPPR DGFFGNPRKS KLICQATGFS PRQIQVSWLR |
| 151/274 | EGKQVGSGVT TDQVQAEAKE SGPTTYKVTS TLTIKESDWL XQSMFTCRVD |
| 201/324 | HRGLTFQQNA SSMCVPDQDT AIRVFAIPPS FASIFLTKST KLTCLVTDLT |
| 251/374 | TYDSVTISWT RQNGEAVKTH TNISESHPNA TFSAVGEASI CEDDWNSGER |
| 301/424 | FTCTVTHTDL PSPLKQTISR PKGVALHRPD VYLLPPAREQ LNLRESATIT |
| 351/474 | CLVTGFSPAD VFVQWMQRGQ PLSPEKYVTS APMPEPQAPG RYFAHSILTV |
| 401/524 | SEEEWNTGET YTCVVAHEAL PNRVTERTVD KSTGKPTLYN VSLVMSDTAG |
| 451/574 | TCY |

Binding molecules, e.g., antibodies, antibody-like molecules, antigen-binding fragments, variants, or derivatives thereof, and/or multimerizing fragments thereof include, but are not limited to, polyclonal, monoclonal, human, humanized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VL or VH domain, fragments produced by a Fab expression library. scFv molecules are known in the art and are described, e.g., in U.S. Pat. No. 5,892,019.

By “specifically binds,” it is generally meant that a binding molecule, e.g., an antibody or fragment, variant, or derivative thereof binds to an epitope via its antigen-binding domain, and that the binding entails some complementarity between the antigen-binding domain and the epitope. According to this definition, a binding molecule, e.g., antibody or antibody-like molecule, is said to “specifically bind” to an epitope when it binds to that epitope, via its antigen-binding domain more readily than it would bind to a random, unrelated epitope. The term “specificity” is used herein to qualify the relative affinity by which a certain binding molecule binds to a certain epitope. For example, binding molecule “A” can be deemed to have a higher specificity for a given epitope than binding molecule “B,” or binding molecule “A” can be said to bind to epitope “C” with a higher specificity than it has for related epitope “D.”

A binding molecule, e.g., an antibody or fragment, variant, or derivative thereof disclosed herein can be said to bind a target antigen with an off rate (k(off)) of less than or equal to 5×10⁻² sec⁻¹, 10⁻² sec⁻¹, 5×10⁻³ sec⁻¹, 10⁻³ sec⁻¹, 5×10⁻⁴ sec⁻¹, 10⁻⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹.

A binding molecule, e.g., an antibody or antigen-binding fragment, variant, or derivative disclosed herein can be said to bind a target antigen with an on rate (k(on)) of greater than or equal to 10³ M⁻¹ sec⁻¹, 5×10³ M⁻¹ sec⁻¹, 10⁴ M⁻¹ sec⁻¹, 5×10⁴ M⁻¹ sec⁻¹, 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

A binding molecule, e.g., an antibody or fragment, variant, or derivative thereof is said to competitively inhibit binding of a reference antibody or antigen-binding fragment to a given epitope if it preferentially binds to that epitope to the extent that it blocks, to some degree, binding of the reference antibody or antigen-binding fragment to the epitope. Competitive inhibition can be determined by any method known in the art, for example, competition ELISA assays. A binding molecule can be said to competitively inhibit binding of the reference antibody or antigen-binding fragment to a given epitope by at least 90%, at least 80%, at least 70%, at least 60%, or at least 50%.

As used herein, the term “affinity” refers to a measure of the strength of the binding of an individual epitope with one or more antigen-binding domains, e.g., of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of antigen-binding domains and an antigen. See, e.g., Harlow at pages 29-34. Avidity is related to both the affinity of individual antigen-binding domains in the population with specific epitopes, and the valencies of the immunoglobulins and the antigen. For example, the interaction between a bivalent monoclonal antibody and an antigen with a highly repeating epitope structure, such as a polymer, would be one of high avidity. An interaction between a bivalent monoclonal antibody with a receptor present at a high density on a cell surface would also be of high avidity.

Binding molecules, e.g., antibodies or fragments, variants, or derivatives thereof as disclosed herein can also be described or specified in terms of their cross-reactivity. As used herein, the term “cross-reactivity” refers to the ability of a binding molecule, e.g., an antibody or fragment, variant, or derivative thereof, specific for one antigen, to react with a second antigen; a measure of relatedness between two different antigenic substances. Thus, a binding molecule is cross reactive if it binds to an epitope other than the one that induced its formation. The cross-reactive epitope generally contains many of the same complementary structural features as the inducing epitope, and in some cases, can actually fit better than the original.

A binding molecule, e.g., an antibody or fragment, variant, or derivative thereof can also be described or specified in terms of their binding affinity to an antigen. For example, a binding molecule can bind to an antigen with a dissociation constant or K_{D} no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

“Antigen-binding antibody fragments” including single-chain antibodies or other antigen-binding domains can exist alone or in combination with one or more of the following: hinge region, CH1, CH2, CH3, or CH4 domains, J-chain, or secretory component. Also included are antigen-binding fragments that can include any combination of variable region(s) with one or more of a hinge region, CH1, CH2, CH3, or CH4 domains, a J-chain, or a secretory component. Binding molecules, e.g., antibodies, or antigen-binding fragments thereof can be from any animal origin including birds and mammals. The antibodies can be, e.g., human, murine, donkey, rabbit, goat, guinea pig, camel, llama, horse, or chicken antibodies. In another embodiment, the variable region can be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and can in some instances express endogenous immunoglobulins and some not, as described infra and, for example in, U.S. Pat. No. 5,939,598 by Kucherlapati et al. According to embodiments of the present disclosure, an IgM or IgM-like antibody or IgM-derived binding molecule as provided herein can include an antigen-binding fragment of an antibody, e.g., a scFv fragment, so long as the IgM or IgM-like antibody is able to form a multimer, e.g., a hexamer or a pentamer.

As used herein, the term “heavy chain subunit” includes amino acid sequences derived from an immunoglobulin heavy chain, a binding molecule, e.g., an antibody or antibody-like molecule comprising a heavy chain subunit can include at least one of: a VH domain, a CH1 domain, a hinge (e.g., upper, middle, and/or lower hinge region) domain, a CH2 domain, a CH3 domain, a CH4 domain, a tail-piece (tp), or a variant or fragment thereof. For example, a binding molecule, e.g., an antibody, antibody-like molecule, or fragment, variant, or derivative thereof can include without limitation, in addition to a VH domain: any combination of a CH1 domain, a hinge, a CH2 domain, a CH3 domain, a CH4 domain or a tailpiece (tp) of one or more antibody isotypes and/or species. In certain embodiments a binding molecule, e.g., an antibody, antibody-like molecule, or fragment, variant, or derivative thereof can include, in addition to a VH domain, a CH3 domain and a CH4-tp domain; or a CH3 domain, a CH4-tp domain, and a J-chain. Further, a binding molecule, e.g., antibody or antibody-like molecule, for use in the disclosure can lack certain constant region portions, e.g., all or part of a CH2 domain. These domains (e.g., the heavy chain subunit) can be modified such that they vary in amino acid sequence from the original immunoglobulin molecule. According to embodiments of the present disclosure, an IgM or IgM-like antibody as provided herein includes sufficient portions of an IgM heavy chain constant region to allow the IgM or IgM-like antibody to form a multimer, e.g., a hexamer or a pentamer, e.g., the IgM heavy chain constant region includes a “multimerizing fragment” of an IgM heavy chain constant region.

As used herein, the term “light chain subunit” includes amino acid sequences derived from an immunoglobulin light chain. The light chain subunit includes at least a VL, and can further include a CL (e.g., CK or CX) domain.

Binding molecules, e.g., antibodies, antibody-like molecules, antigen-binding fragments, variants, or derivatives thereof, or multimerizing fragments thereof can be described or specified in terms of the epitope(s) or portion(s) of an antigen that they recognize or specifically bind. The portion of a target antigen that specifically interacts with the antigen-binding domain of an antibody is an “epitope,” or an “antigenic determinant.” A target antigen can comprise a single epitope or at least two epitopes, and can include any number of epitopes, depending on the size, conformation, and type of antigen.

As used herein, the term “hinge region” includes the portion of a heavy chain molecule that joins the CH1 domain to the CH2 domain in IgG, IgA, and IgD heavy chains. This hinge region comprises approximately 25 amino acids and is flexible, thus allowing the two N-terminal antigen-binding regions to move independently.

As used herein the term “disulfide bond” includes the covalent bond formed between two sulfur atoms. The amino acid cysteine comprises a thiol group that can form a disulfide bond or bridge with a second thiol group.

As used herein, the term “chimeric antibody” refers to an antibody in which the immunoreactive region or site is obtained or derived from a first species and the constant region (which can be intact, partial or modified) is obtained from a second species. In some embodiments the target binding region or site will be from a non-human source (e.g. mouse or primate) and the constant region is human.

The terms “multispecific antibody” or “bispecific antibody” refer to an antibody or antibody-like molecule that has antigen-binding domains for two or more different epitopes within a single antibody molecule. Other binding molecules in addition to the canonical antibody structure can be constructed with two binding specificities.

As used herein, the term “engineered antibody” refers to an antibody in which the variable domain in either the heavy and light chain or both is altered by at least partial replacement of one or more amino acids in either the CDR or framework regions. In certain embodiments entire CDRs from an antibody of known specificity can be grafted into the framework regions of a heterologous antibody. Although alternate CDRs can be derived from an antibody of the same class or even subclass as the antibody from which the framework regions are derived, CDRs can also be derived from an antibody of different class, e.g., from an antibody from a different species. An engineered antibody in which one or more “donor” CDRs from a non-human antibody of known specificity are grafted into a human heavy or light chain framework region is referred to herein as a “humanized antibody.” In certain embodiments not all the CDRs are replaced with the complete CDRs from the donor variable region and yet the antigen-binding capacity of the donor can still be transferred to the recipient variable domains. Given the explanations set forth in, e.g., U.S. Pat. Nos. 5,585,089, 5,693,761, 5,693,762, and 6,180,370, it will be well within the competence of a person or ordinary skill in the art, by carrying out routine experimentation, to obtain a functional engineered or humanized antibody.

As used herein the term “engineered” includes manipulation of nucleic acid or polypeptide molecules by synthetic means (e.g. by recombinant techniques, in vitro peptide synthesis, by enzymatic or chemical coupling of peptides, nucleic acids, or glycans, or some combination of these techniques).

As used herein, the terms “linked,” “fused” or “fusion” or other grammatical equivalents can be used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments can be physically or spatially separated by, for example, in-frame linker sequence. For example, polynucleotides encoding the CDRs of an immunoglobulin variable region can be fused, in-frame, but be separated by a polynucleotide encoding at least one immunoglobulin framework region or additional CDR regions, as long as the “fused” CDRs are co-translated as part of a continuous polypeptide.

In the context of polypeptides, a “linear sequence” or a “sequence” is an order of amino acids in a polypeptide in an amino to carboxyl terminal direction in which amino acids that neighbor each other in the sequence are contiguous in the primary structure of the polypeptide. A portion of a polypeptide that is “amino-terminal” or “N-terminal” to another portion of a polypeptide is that portion that comes earlier in the sequential polypeptide chain. Similarly, a portion of a polypeptide that is “carboxy-terminal” or “C-terminal” to another portion of a polypeptide is that portion that comes later in the sequential polypeptide chain. For example, in a typical antibody, the variable domain is “N-terminal” to the constant region, and the constant region is “C-terminal” to the variable domain.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into RNA, e.g., messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

Terms such as “treating” or “treatment” or “to treat” or “alleviating” or “to alleviate” refer to therapeutic measures that cure, slow down, lessen symptoms of, and/or halt or slow the progression of an existing diagnosed disease, pathologic condition, or disorder. Terms such as “prevent,” “prevention,” “avoid,” “deterrence” and the like refer to prophylactic or preventative measures that prevent the development of an undiagnosed targeted disease, pathologic condition, or disorder. Thus, “a subject in need of treatment” can include subjects already with the disorder; those prone to have the disorder; and those in whom the disorder is to be prevented.

As used herein the terms “serum half-life” or “plasma half-life” refer to the time it takes (e.g., in minutes, hours, or days) following administration for the serum or plasma concentration of a protein or a drug, e.g., a binding molecule such as an antibody, antibody-like molecule or fragment thereof as described herein, to be reduced by 50%. Two half-lives can be described: the alpha half-life, a half-life, or t_{1/2}α, which is the rate of decline in plasma concentrations due to the process of drug redistribution from the central compartment, e.g., the blood in the case of intravenous delivery, to a peripheral compartment (e.g., a tissue or organ), and the beta half-life, β half-life, or t_{1/2}β which is the rate of decline due to the processes of excretion or metabolism.

As used herein the term “area under the plasma drug concentration-time curve” or “AUC” reflects the actual body exposure to drug after administration of a dose of the drug and is expressed in mg*h/L. This area under the curve is measured from time 0 (t₀) to infinity (∞) and is dependent on the rate of elimination of the drug from the body and the dose administered.

As used herein, the term “mean residence time” or “MRT” refers to the average length of time the drug remains in the body.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, swine, cows, bears, and so on.

As used herein, phrases such as “a subject that would benefit from therapy” and “an animal in need of treatment” refers to a subset of subjects, from amongst all prospective subjects, which would benefit from administration of a given therapeutic agent, e.g., a binding molecule such as an antibody or antibody-like molecule, comprising one or more antigen-binding domains. Such binding molecules, e.g., antibodies or antibody-like molecules, can be used, e.g., for a diagnostic procedure and/or for treatment or prevention of a disease.

IgM Antibodies, IgM-Like Antibodies, and IgM-Derived Binding Molecules

IgM is the first immunoglobulin produced by B cells in response to stimulation by antigen and is naturally present at around 1.5 mg/ml in serum with a half-life of about 5 days. IgM is a pentameric or hexameric molecule and thus includes five or six binding units. An IgM binding unit typically includes two light and two heavy chains. While an IgG heavy chain constant region contains three heavy chain constant domains (CH1, CH2 and CH3), the heavy (μ) constant region of IgM additionally contains a fourth constant domain (CH4) and includes a C-terminal “tailpiece” (tp). While several human alleles exist, the human IgM constant region typically comprises the amino acid sequence SEQ ID NO: 22 (IMGT allele IGHM*03, identical to, e.g., GenBank Accession No. pir∥37768) or SEQ ID NO: 23 (IMGT allele IGHM*04, identical to, e.g., GenBank Accession No. sp|P01871.4). The human Cμ 1 region ranges from about amino acid 5 to about amino acid 102 of SEQ ID NO: 22 or SEQ ID NO: 23; the human Cμ2 region ranges from about amino acid 114 to about amino acid 205 of SEQ ID NO: 22 or SEQ ID NO: 23, the human Cμ3 region ranges from about amino acid 224 to about amino acid 319 of SEQ ID NO: 22 or SEQ ID NO: 23, the Cμ 4 region ranges from about amino acid 329 to about amino acid 430 of SEQ ID NO: 22 or SEQ ID NO: 23, and the tailpiece ranges from about amino acid 431 to about amino acid 453 of SEQ ID NO: 22 or SEQ ID NO: 23.

Other forms of the human IgM constant region with minor sequence variations exist, including, without limitation, GenBank Accession Nos. CAB37838.1 and pir∥MHHU. The amino acid substitutions, insertions, and/or deletions at positions corresponding to SEQ ID NO: 22 or SEQ ID NO: 23 described and claimed elsewhere in this disclosure can likewise be incorporated into alternate human IgM sequences, as well as into IgM constant region amino acid sequences of other species.

Each IgM heavy chain constant region can be associated with an antigen-binding domain, e.g., a scFv or VHH, or a subunit of an antigen-binding domain, e.g., a VH region.

Five IgM binding units can form a complex with an additional small polypeptide chain (the J-chain), or a functional fragment, variant, or derivative thereof, to form a pentameric IgM antibody or IgM-like antibody. The precursor form of the human J-chain is presented as SEQ ID NO:1. The signal peptide (underlined) extends from amino acid 1 to about amino acid 22 of SEQ ID NO: 1, and the mature human J-chain extends from about amino acid 23 to amino acid 159 of SEQ ID NO: 1. The mature human J-chain has the amino acid sequence SEQ ID NO: 2.

Exemplary variant and modified J-chains are provided elsewhere herein. Without the J-chain, an IgM antibody or IgM-like antibody typically assembles into a hexamer, comprising six binding units and up to twelve binding unit-associated antigen-binding domains. With a J-chain, an IgM antibody or IgM-like antibody typically assembles into a pentamer, comprising five binding units and up to ten binding unit-associated antigen-binding domains, or more, if the J-chain is a modified J-chain comprising one or more heterologous polypeptides that can be, e.g., additional J-chain-associated antigen-binding domain(s). The assembly of five or six IgM binding units into a pentameric or hexameric IgM antibody or IgM-like antibody is thought to involve interactions between the Cμ4 and tailpiece domains. See, e.g., Braathen, R., et al., J. Biol. Chem. 277:42755-42762 (2002). Accordingly, the constant regions of a pentameric or hexameric IgM antibody or antibody-like molecule provided in this disclosure typically includes at least the Cμ4 and/or tailpiece domains (also referred to herein collectively as Cμ4-tp). A “multimerizing fragment” of an IgM heavy chain constant region thus includes at least the Cμ4-tp domain. An IgM heavy chain constant region can additionally include a Cμ3 domain or a fragment thereof, a Cμ2 domain or a fragment thereof, a Cμ1 domain or a fragment thereof. In certain embodiments, a binding molecule, e.g., an IgM antibody or IgM-like antibody as provided herein can include a complete IgM heavy (μ) chain constant domain, e.g., SEQ ID NO: 22 or SEQ ID NO: 23, or a variant, derivative, or analog thereof, e.g., as provided herein.

In certain embodiments, the disclosure provides a pentameric IgM or IgM-like antibody comprising five bivalent binding units, where each binding unit includes two IgM heavy chain constant regions or multimerizing fragments or variants thereof, each associated with an antigen-binding domain or a subunit of an antigen-binding domain. In certain embodiments, the two IgM heavy chain constant regions are human heavy chain constant regions.

Where the IgM or IgM-like antibody provided herein is pentameric, the IgM or IgM-like antibody typically further includes a J-chain, or functional fragment or variant thereof. As provided herein, in some embodiments, the J-chain is a modified J-chain comprising a J-chain-associated antigen binding domain that specifically binds to an immune effector cell, e.g., a CD8+ cytotoxic T cell or an NK cell. In certain embodiments the modified J-chain can further comprise one or more heterologous moieties attached thereto, e.g., an immune stimulatory agent. In certain embodiments the J-chain can be mutated to affect, e.g., enhance, the serum half-life of the IgM or IgM-like antibody provided herein, as discussed elsewhere herein. In certain embodiments the J-chain can be mutated to affect glycosylation, as discussed elsewhere in this disclosure.

In some embodiments, the multimeric binding molecules are hexameric and comprise six bivalent binding units or variants or fragments thereof. In some embodiments, the multimeric binding molecules are hexameric and comprise six bivalent binding units or variants or fragments thereof, and where each binding unit comprises two IgM heavy chain constant regions or multimerizing fragments or variants thereof.

An IgM heavy chain constant region can include one or more of a Cμ 1 domain or fragment or variant thereof, a Cμ2 domain or fragment or variant thereof, a Cμ3 domain or fragment or variant thereof, a Cμ4 domain or fragment or variant thereof, and/or a tail piece (tp) or fragment or variant thereof, provided that the constant region can serve a desired function in the IgM or IgM-like antibody, e.g., associate with second IgM constant region to form a binding unit with one, two, or more antigen-binding domain(s), and/or associate with other binding units (and in the case of a pentamer, a J-chain) to form a hexamer or a pentamer. In certain embodiments the two IgM heavy chain constant regions or fragments or variants thereof within an individual binding unit each comprise a Cμ4 domain or fragment or variant thereof, a tailpiece (tp) or fragment or variant thereof, or a combination of a Cμ4 domain and a tp or fragment or variant thereof. In certain embodiments the two IgM heavy chain constant regions or fragments or variants thereof within an individual binding unit each further comprise a Cμ3 domain or fragment or variant thereof, a Cμ2 domain or fragment or variant thereof, a Cμ 1 domain or fragment or variant thereof, or any combination thereof.

In some embodiments, the binding units of the IgM or IgM-like antibody comprise two light chains. In some embodiments, the binding units of the IgM or IgM-like antibody comprise two fragments of light chains. In some embodiments, the light chains are kappa light chains. In some embodiments, the light chains are lambda light chains. In some embodiments, each binding unit comprises two immunoglobulin light chains each comprising a VL situated amino terminal to an immunoglobulin light chain constant region.

IgM Antibodies, IgM-Like Antibodies, and IgM-Derived Binding Molecules with Enhanced Serum Half-Life

Certain IgM-derived multimeric bispecific binding molecules provided herein can be modified to have enhanced serum half-life. Exemplary IgM heavy chain constant region mutations that can enhance serum half-life of an IgM-derived binding molecule are disclosed in PCT Publication No. WO 2019/169314A1, which is incorporated by reference herein in its entirety. For example, a variant IgM heavy chain constant region of an IgM-derived binding molecule as provided herein can include an amino acid substitution at an amino acid position corresponding to amino acid S401, E402, E403, R344, and/or E345 of a wild-type human IgM constant region (e.g., SEQ ID NO: 22 or SEQ ID NO: 23). By “an amino acid corresponding to amino acid S401, E402, E403, R344, and/or E345 of a wild-type human IgM constant region” is meant the amino acid in the sequence of the IgM constant region of any species which is homologous to S401, E402, E403, R344, and/or E345 in the human IgM constant region. In certain embodiments, the amino acid corresponding to S401, E402, E403, R344, and/or E345 of SEQ ID NO: 22 or SEQ ID NO: 23 can be substituted with any amino acid, e.g., alanine.

IgM Antibodies, IgM-Like Antibodies, and IgM-Derived Binding Molecules with Reduced CDC Activity

Certain IgM-derived multimeric binding molecules as provided herein can be engineered to exhibit reduced complement-dependent cytotoxicity (CDC) activity to cells in the presence of complement, relative to a reference IgM antibody or IgM-like antibody with a corresponding reference human IgM constant region identical, except for the mutations conferring reduced CDC activity. These CDC mutations can be combined with any of the mutations to block N-linked glycosylation and/or to confer increased serum half-life as provided herein. By “corresponding reference human IgM constant region” is meant a human IgM constant region or portion thereof, e.g., a Cμ3 domain, that is identical to the variant IgM constant region except for the modification or modifications in the constant region affecting CDC activity. In certain embodiments, the variant human IgM constant region includes one or more amino acid substitutions, e.g., in the Cμ3 domain, relative to a wild-type human IgM constant region as described, e.g., in PCT Publication No. WO/2018/187702, which is incorporated herein by reference in its entirety. Assays for measuring CDC are well known to those of ordinary skill in the art, and exemplary assays are described e.g., in PCT Publication No. WO/2018/187702.

In certain embodiments, a variant human IgM constant region conferring reduced CDC activity includes an amino acid substitution corresponding to the wild-type human IgM constant region at position L310, P311, P313, and/or K315 of SEQ ID NO: 22 (human IgM constant region allele IGHM*03) or SEQ ID NO: 23 (human IgM constant region allele IGHM*04). In certain embodiments, a variant human IgM constant region conferring reduced CDC activity includes an amino acid substitution corresponding to the wild-type human IgM constant region at position P311 of SEQ ID NO: 22 or SEQ ID NO: 23. In other embodiments the variant IgM constant region as provided herein contains an amino acid substitution corresponding to the wild-type human IgM constant region at position P313 of SEQ ID NO: 22 or SEQ ID NO: 23. In other embodiments the variant IgM constant region as provided herein contains a combination of substitutions corresponding to the wild-type human IgM constant region at positions P311 of SEQ ID NO: 22 or SEQ ID NO: 23 and/or P313 of SEQ ID NO: 22 or SEQ ID NO: 23. These proline residues can be independently substituted with any amino acid, e.g., with alanine, serine, or glycine. In certain embodiments, a variant human IgM constant region conferring reduced CDC activity includes an amino acid substitution corresponding to the wild-type human IgM constant region at position K315 of SEQ ID NO: 22 or SEQ ID NO: 23. The lysine residue can be independently substituted with any amino acid, e.g., with alanine, serine, glycine, or aspartic acid. In certain embodiments, a variant human IgM constant region conferring reduced CDC activity includes an amino acid substitution corresponding to the wild-type human IgM constant region at position K315 of SEQ ID NO: 22 or SEQ ID NO: 23 with aspartic acid. In certain embodiments, a variant human IgM constant region conferring reduced CDC activity includes an amino acid substitution corresponding to the wild-type human IgM constant region at position L310 of SEQ ID NO: 22 or SEQ ID NO: 23. The lysine residue can be independently substituted with any amino acid, e.g., with alanine, serine, glycine, or aspartic acid. In certain embodiments, a variant human IgM constant region conferring reduced CDC activity includes an amino acid substitution corresponding to the wild-type human IgM constant region at position L310 of SEQ ID NO: 22 or SEQ ID NO: 23 with aspartic acid.

Human and certain non-human primate IgM constant regions typically include five (5) naturally-occurring asparagine (N)-linked glycosylation motifs or sites. As used herein “an N-linked glycosylation motif” comprises or consists of the amino acid sequence N-X₁-S/T, where N is asparagine, X₁ is any amino acid except proline (P), and S/T is serine (S) or threonine (T). The glycan is attached to the nitrogen atom of the asparagine residue. See, e.g., Drickamer K, Taylor M E (2006), Introduction to Glycobiology (2nd ed.). Oxford University Press, USA. N-linked glycosylation motifs occur in the human IgM heavy chain constant regions of SEQ ID NO: 22 or SEQ ID NO: 23 starting at positions 46 (“N1”), 209 (“N2”), 272 (“N3”), 279 (“N4”), and 440 (“N5”). These five motifs are conserved in non-human primate IgM heavy chain constant regions, and four of the five are conserved in the mouse IgM heavy chain constant region. Accordingly, in some embodiments, IgM heavy chain constant regions of a multimeric binding molecule as provided herein comprise 5 N-linked glycosylation motifs: N1, N2, N3, N4, and N5. In some embodiments, at least three of the N-linked glycosylation motifs (e.g., N1, N2, and N3) on each IgM heavy chain constant region are occupied by a complex glycan.

In certain embodiments, at least one, at least two, at least three, or at least four of the N-X₁-S/T motifs can include an amino acid insertion, deletion, or substitution that prevents glycosylation at that motif. In certain embodiments, the IgM-derived multimeric binding molecule can include an amino acid insertion, deletion, or substitution at motif N1, motif N2, motif N3, motif N5, or any combination of two or more, three or more, or all four of motifs N1, N2, N3, or N5, where the amino acid insertion, deletion, or substitution prevents glycosylation at that motif. In some embodiment, the IgM constant region comprises one or more substitutions relative to a wild-type human IgM constant region at positions 46, 209, 272, or 440 of SEQ ID NO: 22 (human IgM constant region allele IGHM*03) or SEQ ID NO: 23 (human IgM constant region allele IGHM*04). See, e.g., U.S. Provisional Application No. 62/891,263, which is incorporated herein by reference in its entirety.

IgA Antibodies, IgA-Like Antibodies, and IgA-Derived Binding Molecules

IgA plays a critical role in mucosal immunity and comprises about 15% of total immunoglobulin produced. IgA can be monomeric or multimeric, forming primarily dimeric molecules, but can also assemble as trimers, tetramers, and/or pentamers. See, e.g., de Sousa-Pereira, P., and J. M. Woof, Antibodies 8:57 (2019).

In some embodiments, the multimeric binding molecules are dimeric and comprise two bivalent binding units or variants or fragments thereof. In some embodiments, the multimeric binding molecules are dimeric, comprise two bivalent binding units or variants or fragments thereof, and further comprise a J-chain or functional fragment or variant thereof as described herein. In some embodiments, the multimeric binding molecules are dimeric, comprise two bivalent binding units or variants or fragments thereof, and further comprise a J-chain or functional fragment or variant thereof as described herein, where each binding unit comprises two IgA heavy chain constant regions or multimerizing fragments or variants thereof.

In some embodiments, the multimeric binding molecules are tetrameric and comprise four bivalent binding units or variants or fragments thereof. In some embodiments, the multimeric binding molecules are tetrameric, comprise four bivalent binding units or variants or fragments thereof, and further comprise a J-chain or functional fragment or variant thereof as described herein. In some embodiments, the multimeric binding molecules are tetrameric, comprise four bivalent binding units or variants or fragments thereof, and further comprise a J-chain or functional fragment or variant thereof as described herein, where each binding unit comprises two IgA heavy chain constant regions or multimerizing fragments or variants thereof.

In certain embodiments, the multimeric binding molecule provided by this disclosure is a dimeric binding molecule that includes IgA heavy chain constant regions, or multimerizing fragments thereof, each associated with an antigen-binding domain for a total of four antigen-binding domains. As provided herein, an IgA antibody, IgA-derived binding molecule, or IgA-like antibody includes two binding units and a J-chain, e.g., a modified J-chain comprising a scFv antibody fragment that binds to CD3, or IL-15 and/or the IL-15 receptor-α sushi domain fused thereto as described elsewhere herein. Each binding unit as provided comprises two IgA heavy chain constant regions or multimerizing fragments or variants thereof. In certain embodiments, at least three or all four antigen-binding domains of the multimeric binding molecule bind to the same target antigen. In certain embodiments, at least three or all four binding polypeptides of the multimeric binding molecule are identical.

A bivalent IgA-derived binding unit includes two IgA heavy chain constant regions, and a dimeric IgA-derived binding molecule includes two binding units. IgA contains the following heavy chain constant domains, Cal (or alternatively CA1 or CH1), a hinge region, Cα2 (or alternatively CA2 or CH2), and Cα3 (or alternatively CA3 or CH3), and a C-terminal “tailpiece.” Human IgA has two subtypes, IgA1 and IgA2. The human IgA1 constant region typically includes the amino acid sequence SEQ ID NO: 24 The human Cα1 domain extends from about amino acid 6 to about amino acid 98 of SEQ ID NO: 24; the human IgA1 hinge region extends from about amino acid 102 to about amino acid 124 of SEQ ID NO: 24, the human Cα2 domain extends from about amino acid 125 to about amino acid 219 of SEQ ID NO: 24, the human Cα3 domain extends from about amino acid 228 to about amino acid 330 of SEQ ID NO: 24, and the tailpiece extends from about amino acid 331 to about amino acid 352 of SEQ ID NO: 24. The human IgA2 constant region typically includes the amino acid sequence SEQ ID NO: 25. The human Cal domain extends from about amino acid 6 to about amino acid 98 of SEQ ID NO: 25; the human IgA2 hinge region extends from about amino acid 102 to about amino acid 111 of SEQ ID NO: 25, the human Cα2 domain extends from about amino acid 113 to about amino acid 206 of SEQ ID NO: 25, the human Cα3 domain extends from about amino acid 215 to about amino acid 317 of SEQ ID NO: 25, and the tailpiece extends from about amino acid 318 to about amino acid 340 of SEQ ID NO: 25.

Two IgA binding units can form a complex with two additional polypeptide chains, the J-chain (e.g., SEQ ID NO: 2) and the secretory component (precursor, SEQ ID NO: 26, mature, SEQ ID NO: 27) to form a bivalent secretory IgA (sIgA)-derived binding molecule as provided herein. The assembly of two IgA binding units into a dimeric IgA-derived binding molecule is thought to involve the Cα3 and tailpiece domains. See. e.g., Braathen, R., et al., J. Biol. Chem. 277:42755-42762(2002). Accordingly, a multimerizing dimeric IgA-derived binding molecule provided in this disclosure typically includes IgA constant regions that include at least the Cα3 and tailpiece domains. Four IgA binding units can likewise form a tetramer complex with a J-chain. A sIgA antibody can also form as a higher order multimer, e.g., a tetramer.

An IgA heavy chain constant region can additionally include a Cα2 domain or a fragment thereof, an IgA hinge region or fragment thereof, a Cα1 domain or a fragment thereof, and/or other IgA (or other immunoglobulin, e.g., IgG) heavy chain domains, including, e.g., an IgG hinge region. In certain embodiments, a binding molecule as provided herein can include a complete IgA heavy (a) chain constant domain (e.g., SEQ ID NO: 24 or SEQ ID NO: 25), or a variant, derivative, or analog thereof. In some embodiments, the IgA heavy chain constant regions or multimerizing fragments thereof are human IgA constant regions.

In certain embodiments each binding unit of a multimeric binding molecule as provided herein includes two IgA heavy chain constant regions or multimerizing fragments or variants thereof, each including at least an IgA Cα3 domain and an IgA tailpiece domain. In certain embodiments the IgA heavy chain constant regions can each further include an IgA Cα2 domain situated N-terminal to the IgA Cα3 and IgA tailpiece domains. For example, the IgA heavy chain constant regions can include amino acids 125 to 353 of SEQ ID NO: 24 or amino acids 113 to 340 of SEQ ID NO: 25. In certain embodiments the IgA heavy chain constant regions can each further include an IgA or IgG hinge region situated N-terminal to the IgA Cα2 domains. For example, the IgA heavy chain constant regions can include amino acids 102 to 353 of SEQ ID NO: 24 or amino acids 102 to 340 of SEQ ID NO: 25. In certain embodiments the IgA heavy chain constant regions can each further include an IgA Cα1 domain situated N-terminal to the IgA hinge region.

In some embodiments, each binding unit of an IgA antibody, IgA-like antibody, or other IgA-derived binding molecule comprises two light chains. In some embodiments, each binding unit of an IgA antibody, IgA-like antibody, or other IgA-derived binding molecule comprises two fragments light chains. In some embodiments, the light chains are kappa light chains. In some embodiments, the light chains are lambda light chains. In some embodiments the light chains are chimeric kappa-lambda light chains. In some embodiments, each binding unit comprises two immunoglobulin light chains each comprising a VL situated amino terminal to an immunoglobulin light chain constant region.

Modified and/or Variant J-Chains

In certain embodiments, the multimeric binding molecule provided herein comprises a J-chain or functional fragment or variant thereof. In certain embodiments, the multimeric binding molecule provided herein is pentameric and comprises a J-chain or functional fragment or variant thereof. In certain embodiments, the multimeric binding molecule provided herein is a dimeric IgA molecule or a pentameric IgM molecule and comprises a J-chain or functional fragment or variant thereof. In some embodiments, the multimeric binding molecule can comprise a naturally occurring J-chain sequence, such as a mature human J-chain sequence (e.g., SEQ ID NO: 2). In some embodiments, the multimeric binding molecule can comprise a functional fragment of a naturally occurring or variant J-chain.

In certain embodiments, the J-chain of a pentameric an IgM or IgM-like antibody or a dimeric IgA or IgA-like antibody as provided herein can be modified, e.g., by introduction of a heterologous moiety, or two or more heterologous moieties, e.g., polypeptides, without interfering with the ability of the IgM or IgM-like antibody or IgA or IgA-like antibody to assemble and bind to its binding target(s). See U.S. Pat. Nos. 9,951,134 and 10,618,978, and in U.S. Patent Application Publication No. US-2019-0185570, each of which is incorporated herein by reference in its entirety. Accordingly, IgM or IgM-like antibodies or IgA or IgA-like antibodies as provided herein, including bispecific or multispecific IgM or IgM-like antibodies or IgA or IgA-like antibodies as described elsewhere herein, can include a modified J-chain or functional fragment or variant thereof that further includes a heterologous moiety, e.g., a heterologous polypeptide, introduced into the J-chain or fragment or variant thereof. In certain embodiments heterologous moiety can be a peptide or polypeptide fused in frame or chemically conjugated to the J-chain or fragment or variant thereof. For example, the heterologous polypeptide can be fused to the J-chain or functional fragment or variant thereof. In certain embodiments, the heterologous polypeptide is fused to the J-chain or functional fragment or variant thereof via a linker, e.g., a peptide linker consisting of least 5 amino acids, but typically no more than 25 amino acids. In certain embodiments, the peptide linker consists of GGGGS (SEQ ID NO: 17), GGGGSGGGGS (SEQ ID NO: 18), GGGGSGGGGSGGGGS (SEQ ID NO: 19), GGGGSGGGGSGGGGSGGGGS (SEQ ID NO: 20), or GGGGSGGGGSGGGGSGGGGSGGGGS (SEQ ID NO: 21). In certain embodiments the heterologous moiety can be a chemical moiety conjugated to the J-chain. Heterologous moieties to be attached to a J-chain can include, without limitation, a binding moiety, e.g., an antibody or antigen-binding fragment thereof, e.g., a single chain Fv (scFv) molecule, a stabilizing peptide that can increase the half-life of the IgM or IgM-like antibody, or a chemical moiety such as a polymer or a cytotoxin. In some embodiments, heterologous moiety comprises a stabilizing peptide that can increase the half-life of the binding molecule, e.g., human serum albumin (HSA) or an HSA binding molecule.

In some embodiments, a modified J-chain includes a J-chain-associated antigen-binding domain, e.g., a polypeptide capable of specifically binding to a target antigen. In certain embodiments, a J-chain-associated antigen-binding domain can be an antibody or an antigen-binding fragment thereof, as described elsewhere herein. In certain embodiments the J-chain-associated antigen-binding domain can be a single chain Fv (scFv) antigen-binding domain or a single-chain antigen-binding domain derived, e.g., from a camelid or condricthoid antibody. The J-chain-associated antigen-binding domain can be introduced into the J-chain at any location that allows the binding of the J-chain-associated antigen-binding domain to its binding target without interfering with J-chain function or the function of an associated IgM or IgA antibody. Insertion locations include but are not limited to at or near the C-terminus, at or near the N-terminus or at an internal location that, based on the three-dimensional structure of the J-chain, is accessible. In certain embodiments, the J-chain-associated antigen-binding domain can be introduced into the mature human J-chain of SEQ ID NO: 2 between cysteine residues 92 and 101 of SEQ ID NO: 2. In a further embodiment, the J-chain-associated antigen-binding domain can be introduced into the human J-chain of SEQ ID NO: 2 at or near a glycosylation site. In a further embodiment, the J-chain-associated antigen-binding domain can be introduced into the human J-chain of SEQ ID NO: 2 within about 10 amino acid residues from the C-terminus, or within about 10 amino acids from the N-terminus. As described elsewhere herein, this disclosure provides a multimeric, bispecific binding molecule comprising a modified J-chain, where the modified J-chain comprises a J-chain-associated antigen binding domain that specifically binds to an immune effector cell, e.g., a T cell such as a CD4+ T cell or a CD8+ cytotoxic T cell or an NK cell.

In some embodiments, a modified J-chain can further include an immune stimulatory agent (ISA), e.g., cytokine, e.g., interleukin-2 (IL-2) or interleukin-15 (IL-15), or a receptor-binding fragment or variant thereof, which in certain embodiments can be associated, either via binding or covalent attachment, to part of its receptor, e.g., the sushi domain of IL-15 receptor-α. Such ISAs are described in detail in co-pending U.S. Provisional Application No. 62/887,458, which is incorporated herein by reference in its entirety.

In certain embodiments, the J-chain of an IgM antibody, IgM-like antibody, IgA antibody, IgA-like antibody, or IgM- or IgA-derived binding molecule as provided herein is a variant J-chain that comprises one or more amino acid substitutions that can alter, e.g., the serum half-life of an IgM antibody, IgM-like antibody, IgA antibody, IgA-like antibody, or IgM- or IgA-derived binding molecule provided herein. For example certain amino acid substitutions, deletions, or insertions can result in the IgM-derived binding molecule exhibiting an increased serum half-life upon administration to a subject animal relative to a reference IgM-derived binding molecule that is identical except for the one or more single amino acid substitutions, deletions, or insertions in the variant J-chain, and is administered using the same method to the same animal species. In certain embodiments the variant J-chain can include one, two, three, or four single amino acid substitutions, deletions, or insertions relative to the reference J-chain.

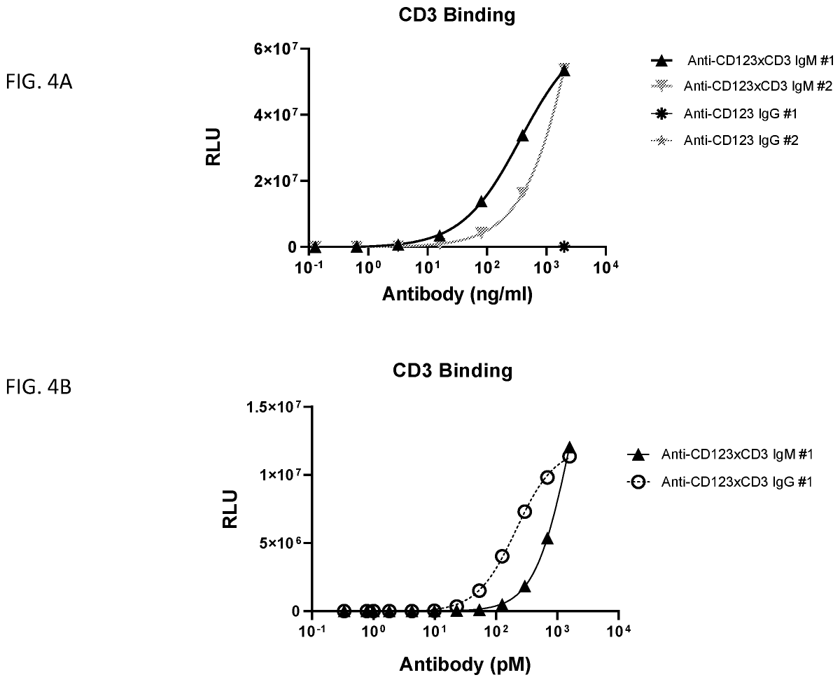

In some embodiments, the multimeric binding molecule can comprise a variant J-chain sequence, such as a variant sequence described herein with reduced glycosylation or reduced binding to one or more polymeric Ig receptors (e.g., pIgR, Fc alpha-mu receptor (FcαμR), or Fc mu receptor (FcμR)). See, e.g., PCT Publication No. WO 2019/169314, which is incorporated herein by reference in its entirety. In certain embodiments, the variant J-chain can comprise an amino acid substitution at the amino acid position corresponding to amino acid Y102 of the mature wild-type human J-chain (SEQ ID NO: 2). By “an amino acid corresponding to amino acid Y102 of the mature wild-type human J-chain” is meant the amino acid in the sequence of the J-chain of any species which is homologous to Y102 in the human J-chain. See PCT Publication No. WO 2019/169314, which is incorporated herein by reference in its entirety. The position corresponding to Y102 in SEQ ID NO: 2 is conserved in the J-chain amino acid sequences of at least 43 other species. See FIG. 4 of U.S. Pat. No. 9,951,134, which is incorporated by reference herein. Certain mutations at the position corresponding to Y102 of SEQ ID NO: 2 can inhibit the binding of certain immunoglobulin receptors, e.g., the human or murine Fcαμ receptor, the murine Fcμ receptor, and/or the human or murine polymeric Ig receptor (pIg receptor) to an IgM pentamer comprising the mutant J-chain. IgM antibodies, IgM-like antibodies, and IgM-derived binding molecules comprising a mutation at the amino acid corresponding to Y102 of SEQ ID NO: 2 have an improved serum half-life when administered to an animal than a corresponding antibody, antibody-like molecule or binding molecule that is identical except for the substitution, and which is administered to the same species in the same manner. In certain embodiments, the amino acid corresponding to Y102 of SEQ ID NO: 2 can be substituted with any amino acid. In certain embodiments, the amino acid corresponding to Y102 of SEQ ID NO: 2 can be substituted with alanine (A), serine (S) or arginine (R). In a particular embodiment, the amino acid corresponding to Y102 of SEQ ID NO: 2 can be substituted with alanine. In a particular embodiment the J-chain or functional fragment or variant thereof is a variant human J-chain and comprises the amino acid sequence SEQ ID NO: 3, a J chain referred to herein as “J*”.

Wild-type J-chains typically include one N-linked glycosylation site. In certain embodiments, a variant J-chain or functional fragment thereof of a multimeric binding molecule as provided herein includes a mutation within the asparagine(N)-linked glycosylation motif N-X₁-S/T, e.g., starting at the amino acid position corresponding to amino acid 49 (motif N6) of the mature human J-chain (SEQ ID NO: 2) or J* (SEQ ID NO: 3), where N is asparagine, X₁ is any amino acid except proline, and S/T is serine or threonine, and where the mutation prevents glycosylation at that motif. As demonstrated in PCT Publication No. WO 2019/169314, mutations preventing glycosylation at this site can result in the multimeric binding molecule as provided herein, exhibiting an increased serum half-life upon administration to a subject animal relative to a reference multimeric binding molecule that is identical except for the mutation or mutations preventing glycosylation in the variant J-chain, and is administered in the same way to the same animal species.

For example, in certain embodiments the variant J-chain or functional fragment thereof of a binding molecule comprising a J-chain as provided herein can include an amino acid substitution at the amino acid position corresponding to amino acid N49 or amino acid S51 of SEQ ID NO: 2 or SEQ ID NO: 3, provided that the amino acid corresponding to S51 is not substituted with threonine (T), or where the variant J-chain comprises amino acid substitutions at the amino acid positions corresponding to both amino acids N49 and S51 of SEQ ID NO: 2 or SEQ ID NO: 3. In certain embodiments, the position corresponding to N49 of SEQ ID NO: 2 or SEQ ID NO: 3 is substituted with any amino acid, e.g., alanine (A), glycine (G), threonine (T), serine (S) or aspartic acid (D). In a particular embodiment, the position corresponding to N49 of SEQ ID NO: 2 or SEQ ID NO: 3 can be substituted with alanine (A). In another particular embodiment, the position corresponding to N49 of SEQ ID NO: 2 or SEQ ID NO: 3 can be substituted with aspartic acid (D). In some embodiments, the position corresponding to S51 of SEQ ID NO: 2 or SEQ ID NO: 3 is substituted with alanine (A) or glycine (G). In some embodiments, the position corresponding to S51 of SEQ ID NO: 2 or SEQ ID NO: 3 is substituted with alanine (A).

Multimeric Bispecific or Multispecific Anti-CD123 Binding Molecules with a Modified J-Chain that Binds to an Immune Effector Cell.

This disclosure provides a multimeric, bispecific or multispecific binding molecule for use in treating cancers, e.g., hematologic cancers, e.g., acute myeloid Leukemia (AML), where the binding molecule is bispecific and targets CD123 (IL-3Rα) on cancer cells with high avidity, while also targeting an immune effector cell, e.g., a CD4+ or CD8+ T cell or an NK cell via a single antigen-binding domain, thereby facilitating effector cell-mediated killing of the cancer cells while at the same time minimizing excessive release of cytokines. In certain aspects the multimeric, bispecific, anti-CD123 binding molecule is an anti-CD123×anti-CD3 binding molecule.

Accordingly, the disclosure provides a multimeric, bispecific or multispecific binding molecule comprising two IgA or IgA-like or five IgM or IgM-like bivalent binding units and a modified J-chain, where the modified J-chain includes at least a wild-type J-chain or a functional fragment or variant thereof and a J-chain-associated antigen-binding domain that specifically binds to an immune effector cell. Each binding unit comprises two antibody heavy chains, each comprising an IgA, IgA-like, IgM, or IgM-like heavy chain constant region or multimerizing fragment thereof (as described elsewhere herein) and at least a heavy chain variable region (VH) portion of a binding unit-associated antigen-binding domain. At least three, at least four, at least five, at least six, at least seven, at least eight, at least nine, or all ten of the binding unit-associated antigen-binding domains specifically bind to CD123. A binding molecule as provided herein can induce immune effector cell-dependent killing of cells, e.g., cancer cells, expressing CD123.

In certain embodiments, the modified J-chain of the binding molecule provided herein includes a variant of a wild-type J-chain or fragment thereof, where the variant includes one or more single amino acid substitutions, deletions, or insertions relative to a wild-type J-chain that can affect serum half-life of the binding molecule; and wherein the binding molecule exhibits an increased serum half-life upon administration to an animal relative to a reference binding molecule that is identical except for the one or more single amino acid substitutions, deletions, or insertions in the J-chain, and is administered in the same way to the same animal species. For example, in certain embodiments the J-chain is a variant human J-chain that comprises the amino acid sequence SEQ ID NO: 3 (“J*”).

In certain embodiments, the J-chain-associated antigen-binding domain of the provided binding molecule comprises an antibody or fragment thereof. In certain embodiments the antibody fragment is a single chain Fv (scFv) fragment. The scFv can be fused or chemically conjugated to the J-chain or fragment or variant, e.g., J*. In certain embodiments, the scFv fragment is fused to the J-chain via a peptide linker e.g., SEQ ID NO: 17-21. As noted elsewhere in the disclosure, the scFv fragment can be fused to J-chain or fragment or variant thereof in any way so long as the function of the J-chain, i.e., to assemble with IgM, IgM-like, IgA, or IgA-like binding units to form a dimer or a pentamer, is not affected. For example the scFv fragment can be fused to the N-terminus of the J-chain or fragment or variant thereof, the C-terminus of the J-chain or fragment or variant thereof, or to both the N-terminus and C-terminus of the J-chain or fragment or variant thereof.

The immune effector cell bound by the antigen binding domain of the modified J-chain can be any immune effector cell confers a beneficial effect when associated with a cancer cell targeted by CD123, for example mediating cell-based killing of the CD123+ cancer cell. In certain embodiments the immune effector cell can be, without limitation, a T cell, e.g., a CD4+ T cell, a CD8+ T cell, an NKT cell, or a 76 T cell, a B cell, a plasma cell, a macrophage, a dendritic cell, or a natural killer (NK) cell. In certain embodiments the immune effector cell is a T cell, e.g., a CD4+ or CD8+ T cell. In certain embodiments the immune effector cell is a CD8+ cytotoxic T cell. In certain embodiments the immune effector cell is an NK cell.

Where the immune effector cell is a T cell, for example a CD8+ T cell, the J-chain-associated scFv fragment can specifically bind to the T cell surface antigen CD3, e.g., CD3. In certain embodiments the anti-CD3ε scFv fragment comprises a heavy chain variable region (VH) and a light chain variable region (VL), wherein the VH comprises the VH complementarity-determining regions VHCDR1, VHCDR2, and VHCDR3 comprising the amino acid sequences SEQ ID NO: 5, SEQ ID NO: 6, and SEQ ID NO: 7, respectively, or SEQ ID NO: 5, SEQ ID NO: 6, or SEQ ID NO: 7 with one, two, or three amino acid substitutions in one or more of the VHCDRs, and wherein the VL comprises the VL complementarity-determining regions VLCDR1, VLCDR2, and VLCDR3 comprising the amino acid sequences SEQ ID NO: 9, SEQ ID NO: 10, and SEQ ID NO: 11, respectively, or SEQ ID NO: 9, SEQ ID NO: 10, and SEQ ID NO: 11 with one, two, or three amino acid substitutions in one or more of the VLCDRs. In certain embodiments, the scFv fragment comprises the VH amino acid sequence SEQ ID NO: 4 and the VL amino acid sequence SEQ ID NO: 8. In other embodiments, the anti-CD3 scFv fragment comprises a heavy chain variable region (VH) and a light chain variable region (VL), wherein the VH and VL comprise the amino acid sequences SEQ ID NO: 13 and SEQ ID NO: 14, respectively. In particular embodiments, the modified J chain comprises an amino acid sequence comprising amino acids 20 to 420 of SEQ ID NO: 12, amino acids 20 to 412 of SEQ ID NO: 15, or amino acids 23 to 415 of SEQ ID NO: 16.

In certain other embodiments, the immune effector cell is an NK cell, and the scFv fragment can specifically bind to CD16 or CD56.

A modified J-chain of a multimeric, bispecific, anti-CD123 binding molecule, e.g., an anti-CD123×anti-CD3 binding molecule as provided herein can be further modified to include additional heterologous moieties attached to the J-chain. Exemplary moieties are described, e.g., in U.S. Pat. No. 9,951,134, and in U.S. Patent Application Publication Nos. US 2019-0185570 and U.S. Pat. No. 10,618,978, and in U.S. Provisional Application No. 62/887,458, all of which are incorporated herein by reference in their entireties. In certain embodiments, the modified J-chain of a multimeric, bispecific anti-CD123 binding molecule, e.g., an anti-CD123×anti-CD3 binding molecule as provided herein can further include an immune stimulatory agent (“ISA”) fused or chemically conjugated to the J-chain or fragment or variant thereof. For example, the ISA can include a cytokine or receptor-binding fragment or variant thereof. In a particular embodiment, a J-chain-associated ISA can include (a) an interleukin-15 (IL-15) protein or receptor-binding fragment or variant thereof (“I”), and (b) an interleukin-15 receptor-α (IL-15Rα) fragment comprising the sushi domain or a variant thereof capable of associating with I (“R”), wherein the J-chain or fragment or variant thereof and at least one of I and R, or both I and R, are associated as a fusion protein, and wherein I and R can associate to function as the ISA. In certain embodiments, the ISA can be fused to the J-chain via a peptide linker.

Anti-CD123 Binding-Unit-Associated Antigen Binding Domains

Each binding unit of an anti-CD123 bispecific, multimeric binding molecule, e.g., an anti-CD123×anti-CD3 binding molecule as provided herein, in addition to two heavy chains, can further include two light chains, where each light chain includes a kappa or lambda light chain constant region, e.g., a human kappa or lambda light chain constant region, and at least a light chain variable region (VL) portion of a binding unit-associated antigen binding domain.

In certain embodiments, the provided multimeric binding molecule is multispecific, e.g., bispecific, trispecific, or tetraspecific, where two or more binding domains associated with the heavy chain constant regions of the binding molecule specifically bind to different targets. In certain embodiments, the binding domains of the multimeric binding molecule all specifically bind to CD123. In certain embodiments, the binding domains of the multimeric binding molecule are identical. In such cases, the multimeric binding molecule can still be bispecific, if, for example, a binding domain with a different specificity is part of a modified J-chain as described elsewhere herein. In certain embodiments, the binding domains are antibody-derived antigen-binding domains, e.g., a scFv associated with the heavy chain constant regions or a VH subunit of an antibody binding domain associated with the heavy chain constant regions.

In addition, an anti-CD123, bispecific, multimeric binding molecule, e.g., an anti-CD123×anti-CD3 binding molecule as provided herein can include at least three, at least four, at least five, at least six, at least seven, at least eight, at least nine, or ten binding unit-associated antigen-binding domains that specifically bind to CD123. In certain embodiments, at least three, at least four, at least five, at least six, at least seven, at least eight, at least nine, or all ten binding unit-associated antigen-binding domains bind to the same CD123 epitope. In certain embodiments, at least three, at least four, at least five, at least six, at least seven, at least eight, at least nine, or all ten binding unit-associated antigen-binding domains are identical. In certain embodiments, all the binding unit-associated antigen binding domains are identical.

In certain embodiments, at least three, at least four, at least five, at least six, at least seven, at least eight, at least nine, or ten binding unit-associated antigen-binding domains of the provided binding molecule include(s) a heavy chain variable region (VH) and a light chain variable region (VL), wherein the VH and VL include six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3 include the CDR amino acid sequences of an antibody that includes the VH and VL amino acid sequences comprising or contained within SEQ ID NO: 32 and SEQ ID NO: 33, SEQ ID NO: 37 and SEQ ID NO: 38, SEQ ID NO: 42 and SEQ ID NO: 43, SEQ ID NO: 44 and SEQ ID NO: 45, SEQ ID NO: 46 and SEQ ID NO: 47, SEQ ID NO: 48 and SEQ ID NO: 49, SEQ ID NO: 50 and SEQ ID NO: 51, SEQ ID NO: 52 and SEQ ID NO: 53, SEQ ID NO: 54 and SEQ ID NO: 55, SEQ ID NO: 56 and SEQ ID NO: 57, SEQ ID NO: 58 and SEQ ID NO: 59, SEQ ID NO: 60 and SEQ ID NO: 61, SEQ ID NO: 62 and SEQ ID NO: 63, SEQ ID NO: 64 and SEQ ID NO: 65, SEQ ID NO: 66 and SEQ ID NO: 67, SEQ ID NO: 68 and SEQ ID NO: 69, SEQ ID NO: 70 and SEQ ID NO: 71, SEQ ID NO: 72 and SEQ ID NO: 73, SEQ ID NO: 74 and SEQ ID NO: 75, SEQ ID NO: 76 and SEQ ID NO: 77, SEQ ID NO: 78 and SEQ ID NO: 79, SEQ ID NO: 80 and SEQ ID NO: 81, SEQ ID NO: 82 and SEQ ID NO: 83, SEQ ID NO: 84 and SEQ ID NO: 85, SEQ ID NO: 86 and SEQ ID NO: 87, SEQ ID NO: 88 and SEQ ID NO: 89, SEQ ID NO: 90 and SEQ ID NO: 91, SEQ ID NO: 92 and SEQ ID NO: 93, SEQ ID NO: 94 and SEQ ID NO: 95, SEQ ID NO: 96 and SEQ ID NO: 97, SEQ ID NO: 98 and SEQ ID NO: 99, SEQ ID NO: 100 and SEQ ID NO: 101, or SEQ ID NO: 102 and SEQ ID NO: 103, respectively, or the CDRs of an antibody that includes the VH and VL amino acid sequences comprising or contained within SEQ ID NO: 32 and SEQ ID NO: 33, SEQ ID NO: 37 and SEQ ID NO: 38, SEQ ID NO: 42 and SEQ ID NO: 43, SEQ ID NO: 44 and SEQ ID NO: 45, SEQ ID NO: 46 and SEQ ID NO: 47, SEQ ID NO: 48 and SEQ ID NO: 49, SEQ ID NO: 50 and SEQ ID NO: 51, SEQ ID NO: 52 and SEQ ID NO: 53, SEQ ID NO: 54 and SEQ ID NO: 55, SEQ ID NO: 56 and SEQ ID NO: 57, SEQ ID NO: 58 and SEQ ID NO: 59, SEQ ID NO: 60 and SEQ ID NO: 61, SEQ ID NO: 62 and SEQ ID NO: 63, SEQ ID NO: 64 and SEQ ID NO: 65, SEQ ID NO: 66 and SEQ ID NO: 67, SEQ ID NO: 68 and SEQ ID NO: 69, SEQ ID NO: 70 and SEQ ID NO: 71, SEQ ID NO: 72 and SEQ ID NO: 73, SEQ ID NO: 74 and SEQ ID NO: 75, SEQ ID NO: 76 and SEQ ID NO: 77, SEQ ID NO: 78 and SEQ ID NO: 79, SEQ ID NO: 80 and SEQ ID NO: 81, SEQ ID NO: 82 and SEQ ID NO: 83, SEQ ID NO: 84 and SEQ ID NO: 85, SEQ ID NO: 86 and SEQ ID NO: 87, SEQ ID NO: 88 and SEQ ID NO: 89, SEQ ID NO: 90 and SEQ ID NO: 91, SEQ ID NO: 92 and SEQ ID NO: 93, SEQ ID NO: 94 and SEQ ID NO: 95, SEQ ID NO: 96 and SEQ ID NO: 97, SEQ ID NO: 98 and SEQ ID NO: 99, SEQ ID NO: 100 and SEQ ID NO: 101, SEQ ID NO: 102 and SEQ ID NO: 103, SEQ ID NO: 107 and SEQ ID NO: 108, SEQ ID NO: 109 and SEQ ID NO: 110, SEQ ID NO: 111 and SEQ ID NO: 112, SEQ ID NO: 113 and SEQ ID NO: 114, SEQ ID NO: 115 and SEQ ID NO: 116, or SEQ ID NO: 117 and SEQ ID NO: 118, respectively, except for one or two amino acid substitutions in one or more of the CDRs. In some embodiments, the HCDR1, HCDR2, HCDR3, LCDR1, LCDR2, and LCDR3 include the CDR amino acid sequences of an antibody that includes the VH and VL amino acid sequences comprising SEQ ID NO: 102 and SEQ ID NO: 103, SEQ ID NO: 113 and SEQ ID NO: 114, or SEQ ID NO: 111 and SEQ ID NO: 112, respectively, with zero, one, or two amino acid substitutions. In some embodiments, the HCDR1, HCDR2, HCDR3, LCDR1, LCDR2, and LCDR3 include the CDR amino acid sequences of an antibody that includes the VH and VL amino acid sequences comprising SEQ ID NO: 113 and SEQ ID NO: 114, respectively, with zero, one, or two amino acid substitutions, such as zero amino acid substitutions.

In certain embodiments, at least three, at least four, at least five, at least six, at least seven, at least eight, at least nine, or ten binding unit-associated antigen-binding domains of the provided binding molecule include(s) an antibody VH and a VL, wherein the VH and VL include the amino acid sequences at least 80%, at least 85%, at least 90%, at least 95% or 100% identical to the mature VH and VL amino acid sequences comprising or contained within SEQ ID NO: 32 and SEQ ID NO: 33, SEQ ID NO: 37 and SEQ ID NO: 38, SEQ ID NO: 42 and SEQ ID NO: 43, SEQ ID NO: 44 and SEQ ID NO: 45, SEQ ID NO: 46 and SEQ ID NO: 47, SEQ ID NO: 48 and SEQ ID NO: 49, SEQ ID NO: 50 and SEQ ID NO: 51, SEQ ID NO: 52 and SEQ ID NO: 53, SEQ ID NO: 54 and SEQ ID NO: 55, SEQ ID NO: 56 and SEQ ID NO: 57, SEQ ID NO: 58 and SEQ ID NO: 59, SEQ ID NO: 60 and SEQ ID NO: 61, SEQ ID NO: 62 and SEQ ID NO: 63, SEQ ID NO: 64 and SEQ ID NO: 65, SEQ ID NO: 66 and SEQ ID NO: 67, SEQ ID NO: 68 and SEQ ID NO: 69, SEQ ID NO: 70 and SEQ ID NO: 71, SEQ ID NO: 72 and SEQ ID NO: 73, SEQ ID NO: 74 and SEQ ID NO: 75, SEQ ID NO: 76 and SEQ ID NO: 77, SEQ ID NO: 78 and SEQ ID NO: 79, SEQ ID NO: 80 and SEQ ID NO: 81, SEQ ID NO: 82 and SEQ ID NO: 83, SEQ ID NO: 84 and SEQ ID NO: 85, SEQ ID NO: 86 and SEQ ID NO: 87, SEQ ID NO: 88 and SEQ ID NO: 89, SEQ ID NO: 90 and SEQ ID NO: 91, SEQ ID NO: 92 and SEQ ID NO: 93, SEQ ID NO: 94 and SEQ ID NO: 95, SEQ ID NO: 96 and SEQ ID NO: 97, SEQ ID NO: 98 and SEQ ID NO: 99, SEQ ID NO: 100 and SEQ ID NO: 101, SEQ ID NO: 102 and SEQ ID NO: 103, SEQ ID NO: 107 and SEQ ID NO: 108, SEQ ID NO: 109 and SEQ ID NO: 110, SEQ ID NO: 111 and SEQ ID NO: 112, SEQ ID NO: 113 and SEQ ID NO: 114, SEQ ID NO: 115 and SEQ ID NO: 116, or SEQ ID NO: 117 and SEQ ID NO: 118, respectively.

In certain embodiments, at least three, at least four, at least five, at least six, at least seven, at least eight, at least nine, or ten binding unit-associated antigen-binding domains of the provided binding molecule include(s) an antibody VH and a VL, wherein the VH and VL include the amino acid sequences at least 80%, at least 85%, at least 90%, at least 95% or 100% identical to the mature VH and VL amino acid sequences comprising SEQ ID NO: 102 and SEQ ID NO: 103, SEQ ID NO: 113 and SEQ ID NO: 114, or SEQ ID NO: 111 and SEQ ID NO: 112, respectively. In certain embodiments, at least three, at least four, at least five, at least six, at least seven, at least eight, at least nine, or ten binding unit-associated antigen-binding domains of the provided binding molecule include(s) an antibody VH and a VL, wherein the VH and VL include the amino acid sequences at least 80%, at least 85%, at least 90%, at least 95% or 100% identical to the mature VH and VL amino acid sequences comprising SEQ ID NO: 113 and SEQ ID NO: 114, respectively, such as 100% identical.

In certain embodiments, at least three, at least four, at least five, at least six, at least seven, at least eight, at least nine, or ten binding unit-associated antigen-binding domains of the provided binding molecule include(s) an antibody VH and a VL, wherein the VH and VL include the amino acid sequences SEQ ID NO: 32 and SEQ ID NO: 33, respectively. In certain embodiments the provided binding molecule is an IgM antibody and each binding unit includes two IgM heavy chains that includes amino acids 20 to 592 of SEQ ID NO: 35 and two kappa light chains that include amino acids 21 to 240 of SEQ ID NO: 36.

In certain embodiments, at least three, at least four, at least five, at least six, at least seven, at least eight, at least nine, or ten binding unit-associated antigen-binding domains of the provided binding molecule include(s) an antibody VH and a VL, wherein the VH and VL include the amino acid sequences SEQ ID NO: 37 and SEQ ID NO: 38, respectively. In certain embodiments the provided binding molecule is an IgM antibody and each binding unit includes two IgM heavy chains that includes amino acids 20 to 589 of SEQ ID NO: 40 and two kappa light chains that include amino acids 21 to 234 of SEQ ID NO: 41.

Polynucleotides, Vectors, and Host Cells

The disclosure further provides a polynucleotide, e.g., an isolated, recombinant, and/or non-naturally occurring polynucleotide, that includes a nucleic acid sequence that encodes a polypeptide subunit of an anti-CD123 multimeric, bispecific binding molecule, e.g., an anti-CD123×anti-CD3 binding molecule as provided herein. By “polypeptide subunit” is meant a portion of a binding molecule, binding unit, IgM antibody, IgM-like antibody, IgA antibody, or IgA-like antibody, J-chain, modified J-chain, or antigen-binding domain that can be independently translated. Examples include, without limitation, an antibody variable domain, e.g., a VH or a VL, a J chain, including modified J-chains as provided herein, a secretory component, a single chain Fv, an antibody heavy chain, an antibody light chain, an antibody heavy chain constant region, an antibody light chain constant region, and/or any fragment, variant, or derivative thereof.

In certain embodiments, the polypeptide subunit can include an IgM heavy chain constant region or IgM-like heavy chain constant region or multimerizing fragment thereof, or an IgA heavy chain constant region or IgA-like heavy chain constant region or multimerizing fragment thereof, which can be fused to an antigen-binding domain or a subunit thereof, e.g., to the VH portion of an antigen-binding domain or the VL portion of an antigen binding domain, all as provided herein. In certain embodiments the polynucleotide can encode a polypeptide subunit that includes a human IgM heavy chain constant region, a human IgM-like heavy chain constant region, a human IgA heavy chain constant region, a human IgA-like heavy chain constant region, or multimerizing fragment thereof, e.g., SEQ ID NO: 22, SEQ ID NO: 23, SEQ ID NO: 24, or SEQ ID NO: 25, any of which can be fused to an antigen-binding domain or subunit thereof, e.g., the C-terminal end of a VH.

In certain embodiments the VH can include HCDR1, HCDR2, and HCDR3 regions that include the CDR amino acid sequences contained in the VH amino acid sequence comprising or contained within SEQ ID NO: 32, SEQ ID NO: 37, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102; SEQ ID NO: 107, SEQ ID NO: 109, SEQ ID NO: 111, SEQ ID NO: 113, SEQ ID NO: 115, or SEQ ID NO: 117, or the CDR amino acid sequences contained in the VH amino acid sequence comprising or contained within SEQ ID NO: 32, SEQ ID NO: 37, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 107, SEQ ID NO: 109, SEQ ID NO: 111, SEQ ID NO: 113, SEQ ID NO: 115, or SEQ ID NO: 117 except for one or two single amino acid substitutions in one or more of the HCDRs. In certain embodiments the VH can include an amino acid sequence at least 80%, at least 85%, at least 90%, at least 95% or 100% identical to the mature VH amino acid sequence comprising or contained within SEQ ID NO: 32, SEQ ID NO: 37, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102. SEQ ID NO: 107, SEQ ID NO: 109, SEQ ID NO: 111, SEQ ID NO: 113, SEQ ID NO: 115, or SEQ ID NO: 117.

In certain embodiments, the polypeptide subunit can include an antibody VL portion of an antigen-binding domain as described elsewhere herein. In certain embodiments the polypeptide subunit can include an antibody light chain constant region, e.g., a human antibody light chain constant region, or fragment thereof, which can be fused to the C-terminal end of a VL.

In certain embodiments the VL can include LCDR1, LCDR2, and LCDR3 regions that include the CDR amino acid sequences contained in the VL amino acid sequence comprising or contained within SEQ ID NO: 33, SEQ ID NO: 38, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 108, SEQ ID NO: 110, SEQ ID NO: 112, SEQ ID NO: 114, SEQ ID NO: 116, or SEQ ID NO: 118, or the CDR amino acid sequences contained in the VL amino acid sequence comprising or contained within SEQ ID NO: 33, SEQ ID NO: 38, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 108, SEQ ID NO: 110, SEQ ID NO: 112, SEQ ID NO: 114, SEQ ID NO: 116, or SEQ ID NO: 118 except for one or two single amino acid substitutions in one or more of the LCDRs. In certain embodiments the VH can include an amino acid sequence at least 80%, at least 85%, at least 90%, at least 95% or 100% identical to the mature VL amino acid sequence comprising or contained within SEQ ID NO: 33, SEQ ID NO: 38, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 108, SEQ ID NO: 110, SEQ ID NO: 112, SEQ ID NO: 114, SEQ ID NO: 116, or SEQ ID NO: 118.

In certain embodiments, the polypeptide subunit can be a modified J-chain as described elsewhere herein. For example, the polypeptide subunit can include an amino acid sequence at least 80%, 85%, 90%, 95%, or 100% identical to amino acids 20 to 420 of SEQ ID NO: 12, amino acids 20 to 412 of SEQ ID NO: 15, or amino acids 23 to 415 of SEQ ID NO: 16.

In certain embodiments, this disclosure provides a composition comprising two, three, or more polynucleotides as provided herein, where the polynucleotides together can encode a multimeric, bispecific anti-CD123 binding molecule, e.g., an anti-CD123×anti-CD3 binding molecule as provided herein. In certain embodiments the polynucleotides can be situated on separate vectors. In certain embodiments two or more of the polynucleotides can be situated on the same vector. Such vectors are likewise provided by the disclosure.

In certain embodiments a polynucleotide as provided herein is situated on an expression vector such as a plasmid, and can include a nucleic acid sequence encoding one polypeptide subunit, e.g., an IgM heavy chain or IgM-like heavy chain, an IgA heavy chain or IgA-like heavy chain, a light chain, or a J-chain, e.g., a modified J-chain, or can include two or more nucleic acid sequences encoding two or more or all three polypeptide subunits of a binding molecule as provided herein. Alternatively, the nucleic acid sequences encoding the three polypeptide subunits can be on separate polynucleotides, e.g., separate expression vectors. The disclosure provides such single or multiple expression vectors. The disclosure also provides one or more host cells encoding the provided polynucleotide(s) or expression vector(s).

The disclosure further provides a host cell, e.g., a prokaryotic or eukaryotic host cell, that includes a polynucleotide or two or more polynucleotides encoding a multimeric, bispecific, anti-CD123 binding molecule, e.g., an anti-CD123×anti-CD3 binding molecule as provided herein, or any subunit thereof, a polynucleotide composition as provided herein, or a vector or two, three, or more vectors that collectively encode the binding molecule as provided herein, or any subunit thereof.

In a related embodiment, the disclosure provides a method of producing a multimeric binding molecule as provided by this disclosure, where the method comprises culturing a host cell as provided herein and recovering the multimeric binding molecule.

Methods of Use

The disclosure further provides a method of treating a disease or disorder, e.g., cancer or other malignancy, e.g., a hematologic cancer or malignancy, in a subject in need of treatment, comprising administering to the subject a therapeutically effective amount of a multimeric, bispecific, anti-CD123 binding molecule, e.g., an anti-CD123×anti-CD3 binding molecule as provided herein. By “therapeutically effective dose or amount” or “effective amount” is intended an amount of the binding molecule that when administered brings about a positive response, e.g., killing of tumor cells, in the subject.

In certain embodiments the cancer to be treated can be any cancer in which the malignant cells express or over-express CD123. For example, the cancer can be acute myeloid leukemia (AML), myelodysplastic syndrome (MDS), chronic myeloid leukemia (CML), B-cell acute lymphoblastic leukemia (B-cell ALL), classical Hodgkin's lymphoma, hairy cell leukemia, chronic lymphocytic leukemia (CLL), systemic mastocytosis, or plasmacytoid dendritic cell leukemia.

Effective doses of compositions for treatment of cancer vary depending upon many different factors, including means of administration, target site, physiological state of the subject, whether the subject is human or an animal, other medications administered, and whether treatment is prophylactic or therapeutic. Usually, the subject is a human, but non-human mammals including transgenic mammals can also be treated. Treatment dosages can be titrated using routine methods known to those of skill in the art to optimize safety and efficacy.

The subject to be treated can be any animal, e.g., mammal, in need of treatment, in certain embodiments, the subject is a human subject.

In its simplest form, a preparation to be administered to a subject is the multimeric, bispecific anti-CD123 binding molecule, e.g., an anti-CD123×anti-CD3 binding molecule as provided herein, or a multimeric antigen-binding fragment thereof, administered in conventional dosage form, which can be combined with a pharmaceutical excipient, carrier or diluent as described elsewhere herein.

The compositions of the disclosure can be administered by any suitable method, e.g., parenterally, intraventricularly, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

Pharmaceutical Compositions and Administration Methods

Methods of preparing and administering a multimeric, bispecific anti-CD123 binding molecule, e.g., an anti-CD123×anti-CD3 binding molecule as provided herein to a subject in need thereof are well known to or are readily determined by those skilled in the art in view of this disclosure. The route of administration of can be, for example, intratumoral, oral, parenteral, by inhalation or topical. The term parenteral as used herein includes, e.g., intravenous, intraarterial, intraperitoneal, intramuscular, subcutaneous, rectal, or vaginal administration. While these forms of administration are contemplated as suitable forms, another example of a form for administration would be a solution for injection, in particular for intratumoral, intravenous, or intraarterial injection or drip. A suitable pharmaceutical composition can comprise a buffer (e.g. acetate, phosphate or citrate buffer), a surfactant (e.g. polysorbate), optionally a stabilizer agent (e.g. human albumin), etc.

As discussed herein multimeric, bispecific anti-CD123 binding molecule, e.g., an anti-CD123×anti-CD3 binding molecule as provided herein can be administered in a pharmaceutically effective amount for the treatment of a subject in need thereof. In this regard, it will be appreciated that the disclosed multimeric, bispecific anti-CD123 binding molecule, e.g., an anti-CD123×anti-CD3 binding molecule can be formulated so as to facilitate administration and promote stability of the active agent. Pharmaceutical compositions accordingly can comprise a pharmaceutically acceptable, non-toxic, sterile carrier such as physiological saline, non-toxic buffers, preservatives and the like. A pharmaceutically effective amount of a multimeric binding molecule comprising an ISA as provided herein means an amount sufficient to achieve effective binding to a target and to achieve a therapeutic benefit. Suitable formulations are described in Remington's Pharmaceutical Sciences, e.g., 21^{st} Edition (Lippincott Williams & Wilkins) (2005).

Certain pharmaceutical compositions provided herein can be orally administered in an acceptable dosage form including, e.g., capsules, tablets, aqueous suspensions or solutions. Certain pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents.

The amount of a multimeric, bispecific anti-CD123 binding molecule, e.g., an anti-CD123×anti-CD3 binding molecule that can be combined with carrier materials to produce a single dosage form will vary depending, e.g., upon the subject treated and the particular mode of administration. The composition can be administered as a single dose, multiple doses or over an established period of time in an infusion. Dosage regimens also can be adjusted to provide the optimum desired response (e.g., a therapeutic or prophylactic response).

In keeping with the scope of the present disclosure, a multimeric, bispecific anti-CD123 binding molecule, e.g., an anti-CD123×anti-CD3 binding molecule as provided herein can be administered to a subject in need of therapy in an amount sufficient to produce a therapeutic effect. A multimeric, bispecific anti-CD123 binding molecule, e.g., an anti-CD123×anti-CD3 binding molecule as provided herein can be administered to the subject in a conventional dosage form prepared by combining the antibody or multimeric antigen-binding fragment, variant, or derivative thereof of the disclosure with a conventional pharmaceutically acceptable carrier or diluent according to known techniques. The form and character of the pharmaceutically acceptable carrier or diluent can be dictated by the amount of active ingredient with which it is to be combined, the route of administration and other well-known variables.

This disclosure also provides for the use of a multimeric, bispecific anti-CD123 binding molecule, e.g., an anti-CD123×anti-CD3 binding molecule as provided herein in the manufacture of a medicament for treating, preventing, or managing cancer or other malignancy. The disclosure also provides for multimeric, bispecific anti-CD123 binding molecule, e.g., an anti-CD123×anti-CD3 binding molecule as provided herein for use in treating, preventing, or managing cancer.

This disclosure employs, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Green and Sambrook, ed. (2012) Molecular Cloning A Laboratory Manual (4th ed.; Cold Spring Harbor Laboratory Press); Sambrook et al., ed. (1992) Molecular Cloning: A Laboratory Manual, (Cold Springs Harbor Laboratory, NY); D. N. Glover and B. D. Hames, eds., (1995) DNA Cloning 2d Edition (IRL Press), Volumes 1-4; Gait, ed. (1990) Oligonucleotide Synthesis (IRL Press); Mullis et al. U.S. Pat. No. 4,683,195; Hames and Higgins, eds. (1985) Nucleic Acid Hybridization (IRL Press); Hames and Higgins, eds. (1984) Transcription And Translation (IRL Press); Freshney (2016) Culture Of Animal Cells, 7th Edition (Wiley-Blackwell); Woodward, J., Immobilized Cells And Enzymes (IRL Press) (1985); Perbal (1988) A Practical Guide To Molecular Cloning; 2d Edition (Wiley-Interscience); Miller and Calos eds. (1987) Gene Transfer Vectors For Mammalian Cells, (Cold Spring Harbor Laboratory); S. C. Makrides (2003) Gene Transfer and Expression in Mammalian Cells (Elsevier Science); Methods in Enzymology, Vols. 151-155 (Academic Press, Inc., N.Y.); Mayer and Walker, eds. (1987) Immunochemical Methods in Cell and Molecular Biology (Academic Press, London); Weir and Blackwell, eds.; and in Ausubel et al. (1995) Current Protocols in Molecular Biology (John Wiley and Sons).

General principles of antibody engineering are set forth, e.g., in Strohl, W. R., and L. M. Strohl (2012), Therapeutic Antibody Engineering (Woodhead Publishing). General principles of protein engineering are set forth, e.g., in Park and Cochran, eds. (2009), Protein Engineering and Design (CDC Press). General principles of immunology are set forth, e.g., in: Abbas and Lichtman (2017) Cellular and Molecular Immunology 9th Edition (Elsevier). Additionally, standard methods in immunology known in the art can be followed, e.g., in Current Protocols in Immunology (Wiley Online Library); Wild, D. (2013), The Immunoassay Handbook 4th Edition (Elsevier Science); Greenfield, ed. (2013), Antibodies, a Laboratory Manual, 2d Edition (Cold Spring Harbor Press); and Ossipow and Fischer, eds., (2014), Monoclonal Antibodies: Methods and Protocols (Humana Press).

All of the references cited above, as well as all references cited herein, are incorporated herein by reference in their entireties.

The following examples are offered by way of illustration and not by way of limitation.

### EXAMPLES

### Example 1: Antibody Generation and Purification

Anti-CD123×CD3 IgM #1 and #2 and Anti-CD123 IgG #1 and #2

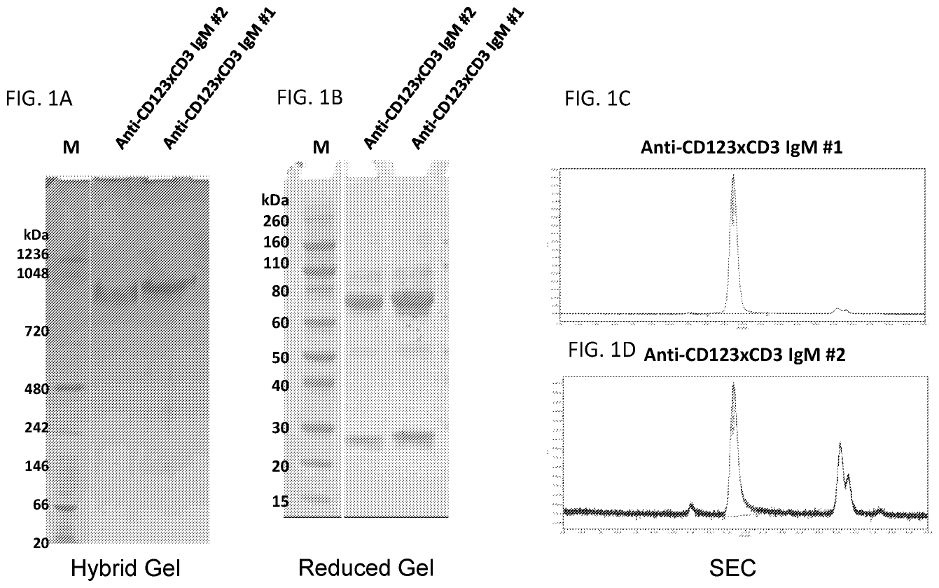

As exemplary constructs, the VH and VL regions of four anti-CD123 antibodies were incorporated into IgM (with SJ* chain, amino acids 20 to 420 of SEQ ID NO: 12 to form bispecific IgM antibodies) and IgG formats according to standard cloning protocols. Anti-CD123 #1 constructs include the VH and VL amino acid sequences SEQ ID NO: 32 and SEQ ID NO: 33, respectively, Anti-CD123 #2 constructs include the VH and VL amino acid sequences SEQ ID NO: 38 and SEQ ID NO: 39, respectively, Anti-CD123 #3 constructs include the VH and VL amino acid sequences SEQ ID NO: 102 and SEQ ID NO: 103, respectively, and Anti-CD123 #4 constructs include the VH and VL amino acid sequences SEQ ID NO: 107 and SEQ ID NO: 108, respectively. These antibody constructs were expressed and purified as described below. The IgM bispecific antibodies (plus modified J-chain-SJ*) were resolved on reduced and non-reduced gels as follows. FIG. 1A shows an exemplary non-reduced gel to resolve high molecular weight IgMs, and FIG. 1B shows an exemplary reduced gel to show IgM heavy and light chains. For the non-reduced gel, samples were mixed with NuPage LDS Sample Buffer (Life Technologies #NP0007) and loaded onto a NativePage Novex 3-12% Bis-Tris Gel (Life Technologies #BN1003). Novex Tris-Acetate SDS Running Buffer (Life Technologies #LA0041) was used for gel electrophoresis, and gel was stained with Colloidal Blue Stain (Life Technologies #LC6025). For the reduced gel, samples were mixed with sample buffer and NuPage reducing agent (Life Technologies #NP0004) and heated to 80° C. for 10 minutes and loaded on a NuPage Novex 4-12% Bis-Tris Gel (Life Technologies #NP0322). NuPage MES SDS Running Buffer (Life Technologies #NP0002) was used for gel electrophoresis and gel was stained with Colloidal Blue.

Additional Anti-CD123×CD3 and Anti CD123 IgG Constructs

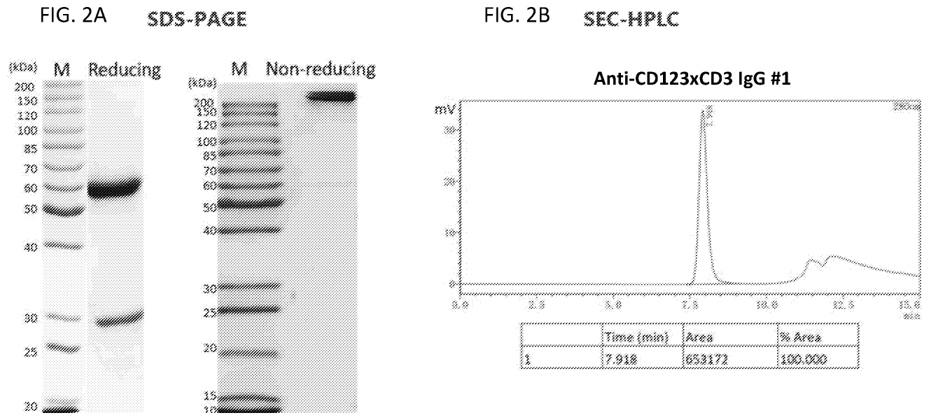

An anti-CD123×anti-CD3 BiTE construct is described in PCT Appl. Publ. No. WO 2017/210443 A1. The construct includes a first heavy chain comprising an anti-CD123 VH sequence (VH=SEQ ID NO: 32; heavy chain=SEQ ID NO: 104), a light chain comprising an anti-CD123 VL sequence (VL=SEQ ID NO: 32, light chain=SEQ ID NO: 105), and a second heavy chain comprising an anti-CD3 scFv fused to an IgG heavy chain constant region (Heavy chain=SEQ ID NO: 106). This construct was synthesized, expressed and purified through commercial vendors (Creative Biolabs and ATUM), and is designated herein as anti-CD123×CD3 IgG #1. The protein was resolved by reduced and non-reduced gels (FIG. 2A) resolution of the purified protein by size exclusion chromatograph shown in FIG. 2B.

Protein Expression and Purification

Transfection. Heavy, light, and modified J chain (SJ*) DNAs (for IgM pentamer constructs) were transfected into, e.g., CHO cells or Expi 293 cells. DNA for expression vectors were mixed with polyethylamine (PEI) reagents (ExpiFectamine™ 293 Transfection Kit) and then added to cells. PEI transfection with CHO-S or 293 expi cells was conducted according to established techniques (see “Biotechnology and Bioengineering, Vol. 87, 553-545”).

IgG expression products were expressed and purified by a commercial vendor.

IgM expression products were purified, e.g. using Capto Core 400 (GE life science) and POROS™ 50 HQ Strong Anion Exchange Resin (Thermo Fisher) according to manufacturer's recommendation. Protein peaks were resolved by size exclusion chromatography as shown in FIG. 1C and FIG. 1D for the IgM expression products.

### Example 2: Antibody Specificity Measured by ELISA

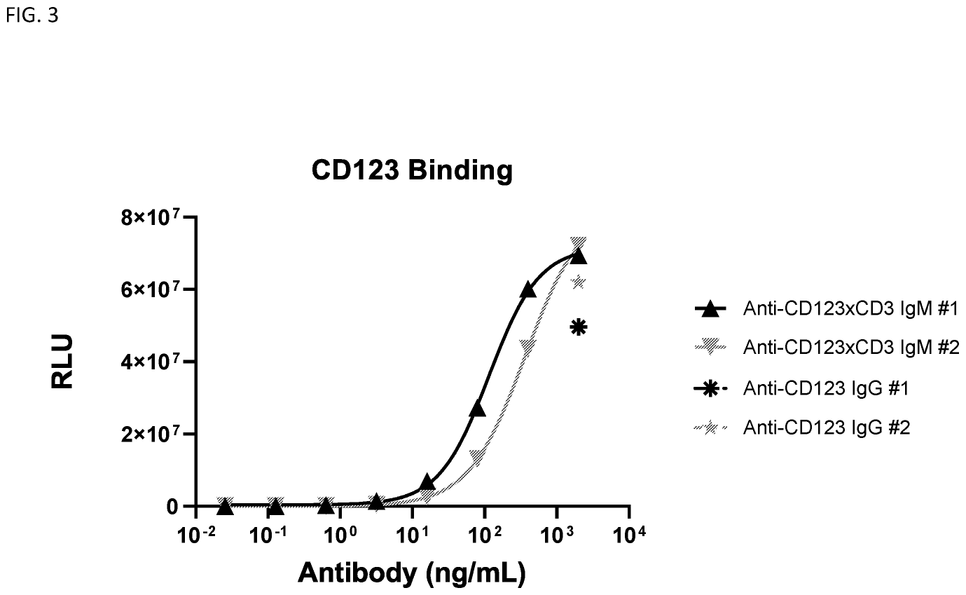

The specificity of the Anti-CD123×CD3 IgM #1 and Anti-CD123×CD3 IgM #2 for human CD123 and CD3ε, as well as the specificity of control Anti-CD123 IgG #1 and Anti CD123 IgG #2 for CD123, and bispecific anti-CD123×CD3 IgG #1 for CD3ε, were measured in ELISA assays as follows. 96-well white polystyrene ELISA plates (Pierce 15042) were coated with 100 μL per well of 0.5 μg/mL recombinant human CD123 protein (Sino Biological 10518-H08H-50) or recombinant human CD3ε protein (Acro Biosystems, CDE-H5256-100) overnight at 4° C. Plates were then washed 5 times with 0.05% PBS-Tween and blocked with 2% BSA-PBS. After blocking, 100 μL of serial dilutions of CD123 IgM or IgG, standards, and controls were added to the wells and incubated at room temperature for 2 hours. The plates were then washed 10 times and incubated with HRP conjugated mouse anti-human kappa (Southern Biotech, 9230-05. 1:6000 diluted in 2% BSA-PBS) for 30 min. After 10 final washes using 0.05% PBS-Tween, the plates were read out using SuperSignal chemiluminescent substrate (ThermoFisher, 37070). Luminescent data were collected on an EnVision plate reader (Perkin-Elmer) and analyzed with GraphPad Prism using a 4-parameter logistic model. Binding of the IgM bispecific antibodies to CD123 is shown in FIG. 3, and binding of the IgM and IgG bispecific antibodies to CD3ε is shown in FIG. 4A-B.

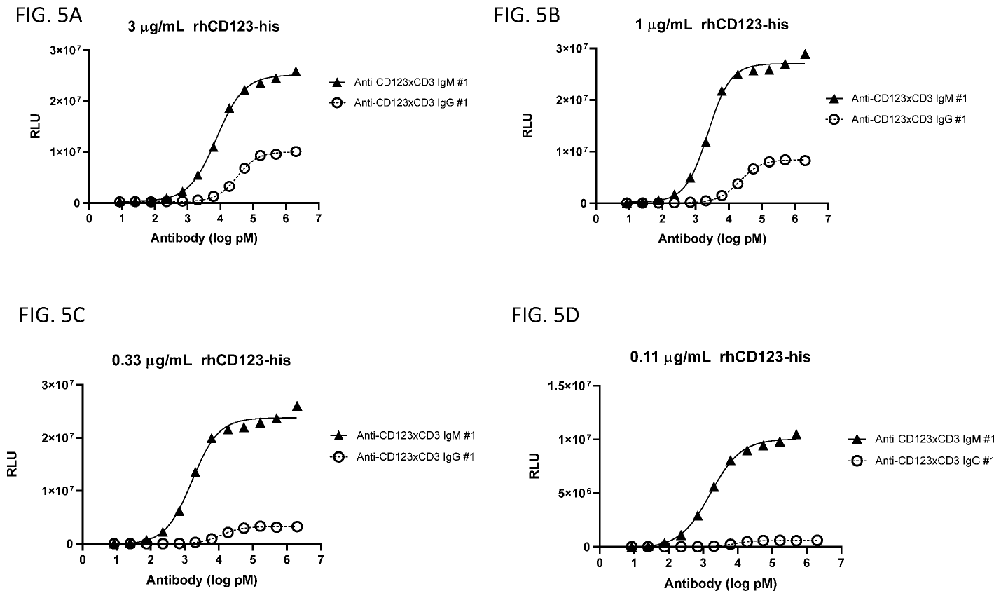

To compare binding of the IgM and IgG bispecific antibodies to CD123 at different protein concentrations, 384 well white plates were coated with 25 μl of different CD123 protein concentrations (3 μg/ml, 1 μg/ml, 0.33 μg/ml and 0.11 μg/ml) for 1 hour at 37° C. The plates were washed and Blocking buffer Starting Block T20 (Thermo, 37539) was used to block for 15 min. 25 μl of serial dilutions of CD123×CD3 IgM or IgG #1 were added to the plates and incubated 30 min at 37° C., washed 10 times, and secondary antibody anti-human Kappa EPR5367-8 HRP conjugated was used to detect bound IgM or IgG (Abcam, ab202549). The results are shown in FIG. 5A-D. The IgM antibody shown superior binding at all concentrations.

### Example 3: Binding to AML Cell Lines

AML Cell Lines-CD123 Surface Quantification

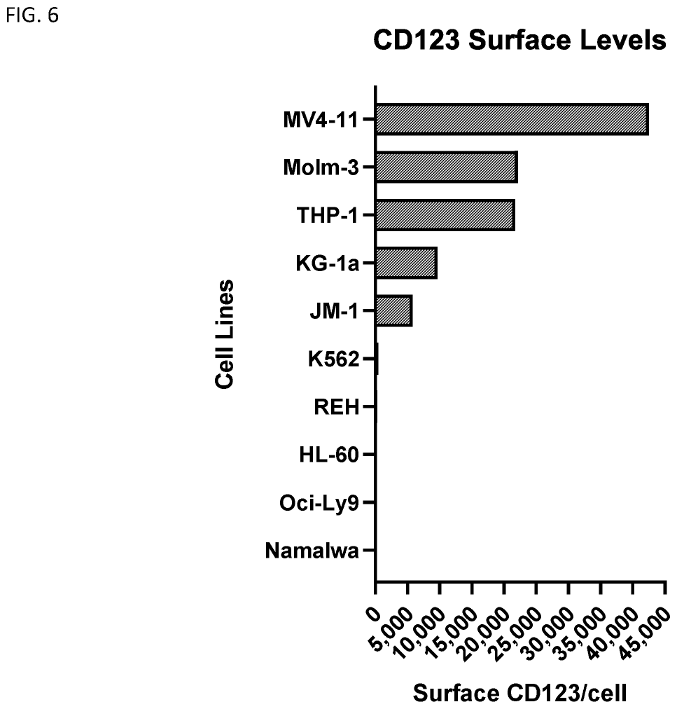

AML cell lines were purchased from ATCC or DSMZ (MV4-11, THP-1, Namalwa, KG-1a, Molm-13, JM-1, REH, K562, HL-60, and Oci-Ly9). Cells were cultured in appropriate media according to seller recommendations. CD123 surface expression was quantified using a commercial anti-CD123 antibody PE-conjugated (Biolegend, Clone 6H6, 306006) and Quantum™ R-PE MESF beads (Bangs Laboratories, 827). The results are shown in FIG. 6. The MV4-11, Molm-3, Thp-1, KG-1a and JM-1 cells expressed detectable levels of CD123.

AML Cell Lines IgM and IgG Binding Assay

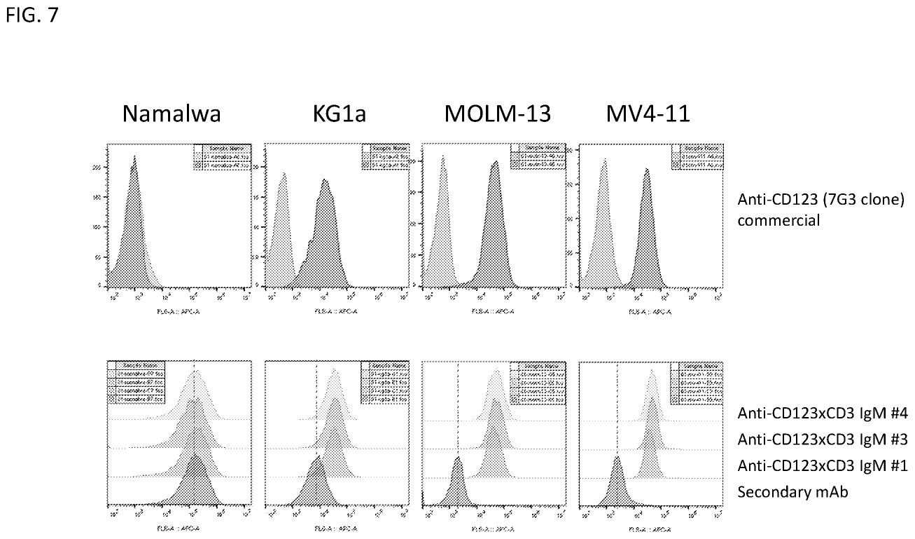

To assess the ability of IgG and IgM antibodies to bind CD123 on AML cells expressing the CD123 protein, a binding assay was performed by the following method. Cells were washed with FACS Stain Buffer (BD Pharmigen Catalog #554656) and pre-incubated with Fc Block (BD, 564220) for 10 minutes at room temperature. 1×10⁵ cells were stained with 1 μg of anti-CD123 antibodies, 1 μg/mL IgG isotype control (Jackson ImmunoResearch #009-000-003), or 1 μg/mL IgM isotype control (Jackson ImmunoResearch #009-000-012) for 30 minutes at 4° C. Cells were washed twice, then stained for 30 minutes at 4° C. with 5 μg/mL anti-human kappa-AF488 secondary antibody (Biolegend #316512). Cells were washed twice, resuspended in FACS Stain Buffer, and acquired by flow cytometry. The results are shown in FIG. 7.

### Example 4: T Cell-Directed AML Cell Killing

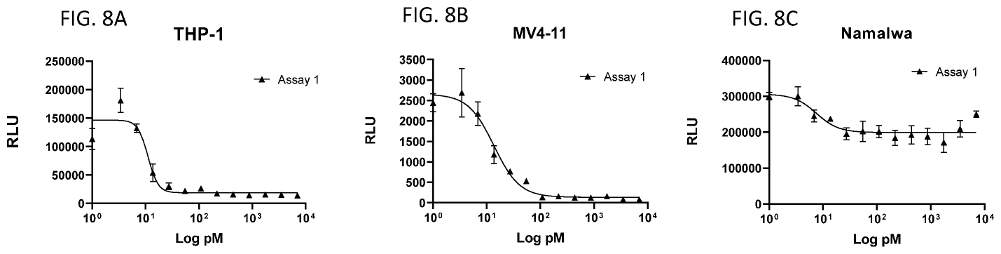

In order to demonstrate that bispecific CD123×CD3 IgM binding molecules can kill target cells in the presence of human T-cells, we performed co-culture experiments. 5×10³ Tumor cells MV4-11, THP-1, and Namalwa (all expressing firefly luciferase) were co-cultured with T cells at different Effector to target (E:T) ratios in the presence of serial dilutions of Anti-CD123×CD3 IgM #1 in 100 μL total volume of AIM-V media supplemented with 3% heat-inactivated fetal bovine serum (FBS) per well on a 96 round bottom tissue culture plate. After 72 or 96 hours of incubation at 37° C. in a 5% CO₂ incubator, 50 μl of supernatant was removed and frozen at −80° C. for later cytokine release analysis. 50 μl of luciferase substrate e.g., ONE-Glo EX Luciferase Assay System, Promega was added to the wells. The plates were shaken briefly to mix the reagents, and luciferase luminescent signal was measured on an EnVision plate reader (Perkin-Elmer). The data was then analyzed with GraphPad Prism to determine the EC₅₀. Representative dose response curves are shown in FIG. 8A-C. THV-1 cells (EC50: 11.12 pM) and MV4-11 cells (EC50: 13.63 pM), which express detectable levels of CD123 were effectively killed, where Namalwa cells, which do not express CD123 were not killed.

### Example 5: Anti-CD123×CD3 IgM #1 Activates CD8+ T Cells but not CD4+ T Cells

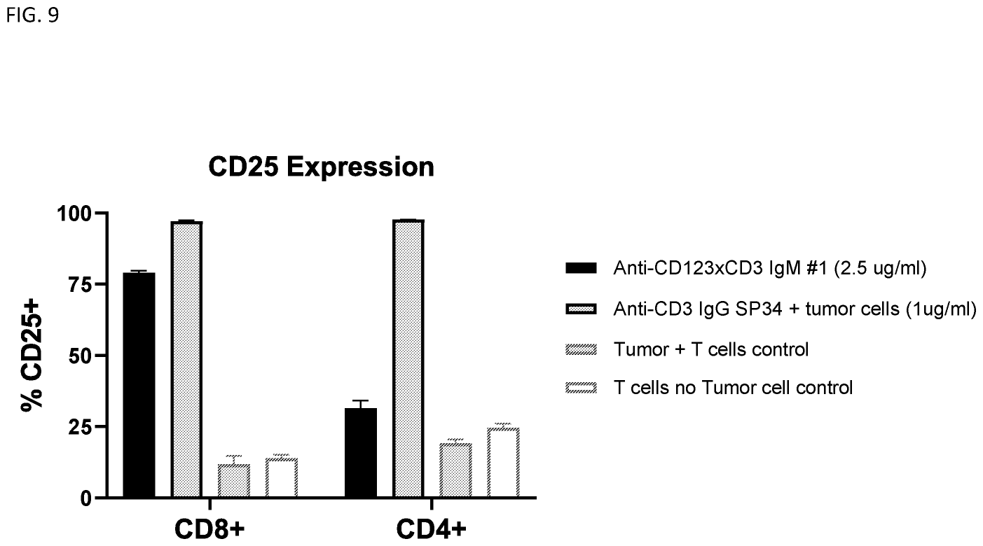

The ability of the Anti-CD123 XCD3 IgM #1 to enhance T cell activation was assessed as follows. Human pan T cells were isolated from PBMCs using MACS pan T cell isolation kit according to manufacturer instructions. T cells were then labeled with cell trace violet dye (Thermo, C34557). 10×10³ MV4-11 cells per well were co-cultured with 40×10³ human Pan T cells in the presence of 2.5 μg/ml anti CD123×CD3 IgM #1, or 1 pg/ml anti CD3 mAb (SP34 ebioscience, Thermo 16-0037-85) for 72 hours. Cells were stained for FACS analysis with the following staining panel: anti-CD8 BV510, anti-CD25 APC, and anti-CD4 BV785 from Biolegend, and the Fixable viability dye LIVE-OR-DYE 750/788 (Biotium, 32008). The results are shown in FIG. 9. Anti-CD123×CD3 IgM #1 enhanced the CD25 activation marker on CD8+ T cells but not on CD4+ T cells.

### Example 6: Cytokine Release

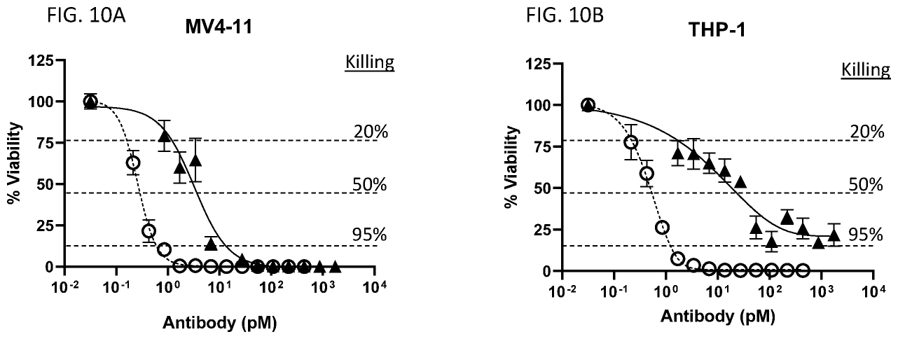

Supernatants from T cell directed cytotoxicity assays performed in Example 4 were collected at time points in which 0%, 20%, 50%, and 95% of the cells were killed. FIGS. 10A and 10B compare anti-CD123×CD3 IgM #1 (triangles) and anti-CD123×CD3 IgG #1 (open circles) in a pan-TDCC assay on MV4-11 cells (panel A) and THP-1 cells (panel B) at the indicated points on the curve. Open circles: anti-CD123×CD3 IgG #1, closed triangles: anti-CD123×CD3 IgM #1. Samples were collected at the indicated levels of killing.

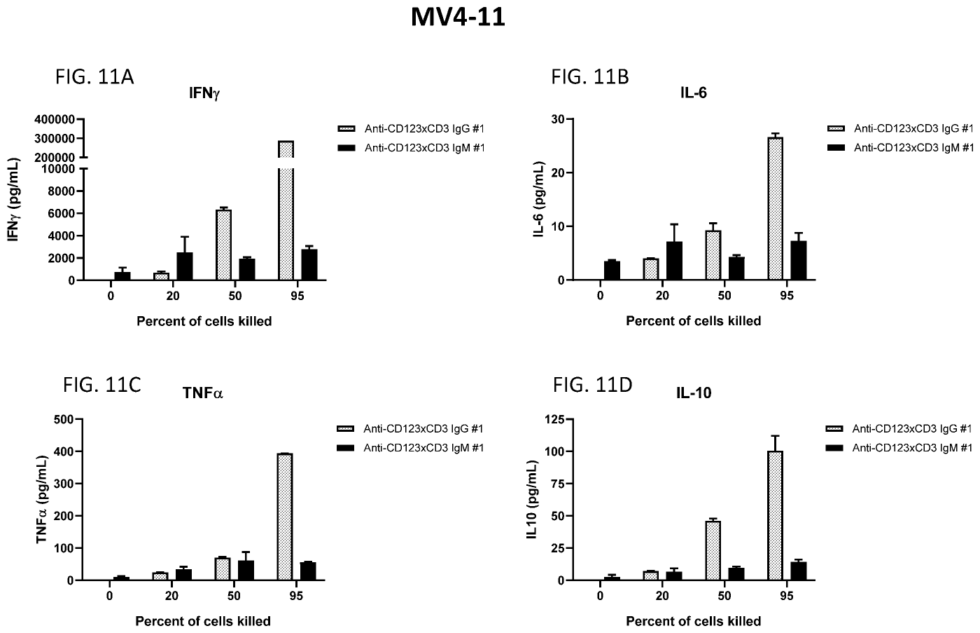

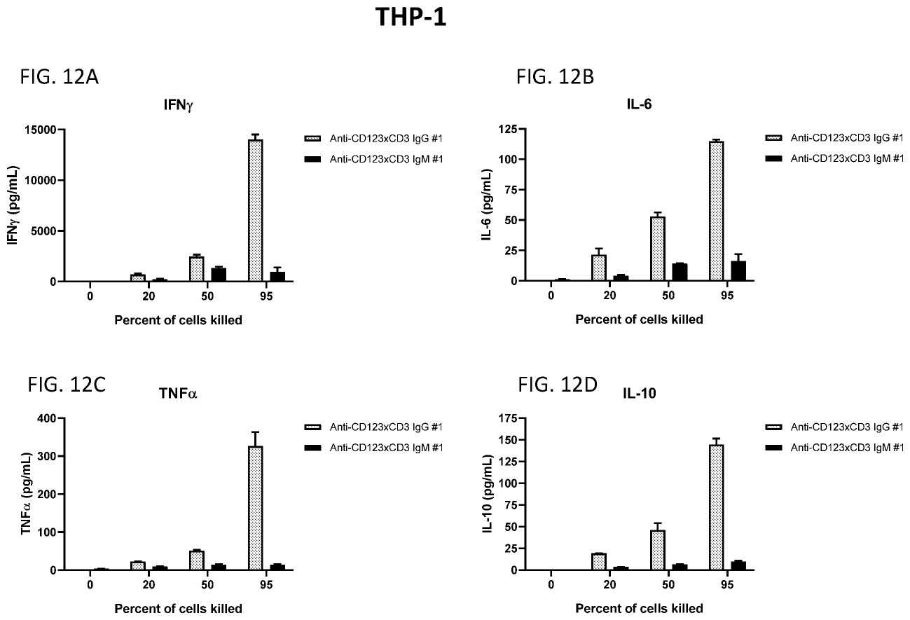

Supernatants from T cell directed cytotoxicity assays performed in Example 4 were collected as indicated and assayed for a panel of cytokines including IFNγ, IL-4, TNF, IL-10, and IL-6 using V-PLEX Proinflammatory Panel human (MSD, K15049D-2) according to manufacturer's protocol. The results were then analyzed with GraphPad Prism. The results for MV4-11 cells are shown in FIGS. 11A-D, and the results for THP cells, both at day 4, are shown in FIGS. 12A-D. Even where 95% of the cells were killed, the IgM construct resulted in minimal cytokine release, while the IgG construct resulted in high levels of cytokine release.

### Example 7: Additional Antibody Generation and Purification

Additional exemplary antibodies as indicated in Table 2 were generated and purified as described in Example 1. The antibodies assembled as pentamers with a J-chain (data not shown).

TABLE 2
Antibodies Generated

| | Heavy Chain | CD123 | CD123 | CD3 | CD3 | J |
|---|---|---|---|---|---|---|
| Name | Mutation | VH | VL | VH | VL | chain |
| IGM #A-a-J*-H1 | | 102 | 103 | 4 | 8 | 3 |
| IGM #A-b-J*-H1 | | 102 | 103 | 119 | 120 | 3 |
| IGM #A-a-J*-H2 | P311A, | 102 | 103 | 4 | 8 | 3 |
| | P313S | | | | | |
| IGM #A-a-J*-H3 | K315D | 102 | 103 | 4 | 8 | 3 |
| IGM #A-a-J*-H4 | L310D | 102 | 103 | 4 | 8 | 3 |
| IGM #A-c-J*-H1 | | 102 | 103 | 125 | 126 | 3 |
| IGM #A-d-J*-H1 | | 102 | 103 | 121 | 122 | 3 |
| IGM #A-e-J*-H1 | | 102 | 103 | 127 | 128 | 3 |
| IGM #A-f-J*-H1 | | 102 | 103 | 13 | 14 | 3 |
| IGM #A-f-JH-H1 | | 102 | 103 | 13 | 14 | 119 |
| IGM #B-b-J*-H1 | | 113 | 114 | 119 | 120 | 3 |
| IGM #B-c-J*-H1 | | 113 | 114 | 125 | 126 | 3 |
| IGM #C-b-J*-H1 | | 111 | 112 | 119 | 120 | 3 |
| IGM #D-a-J*-H1 | | 32 | 33 | 4 | 8 | 3 |
| IGM #D-f-J*-H1 | | 32 | 33 | 13 | 14 | 3 |
| IGM #D-f-JH-H1 | | 32 | 33 | 13 | 14 | 119 |
| IGM #E-a-J*-H1 | | 107 | 108 | 4 | 8 | 3 |
| IGM #F-b-J*-H1 | | 62 | 63 | 119 | 120 | 3 |

### Example 8: Antibody Specificity Measured by ELISA

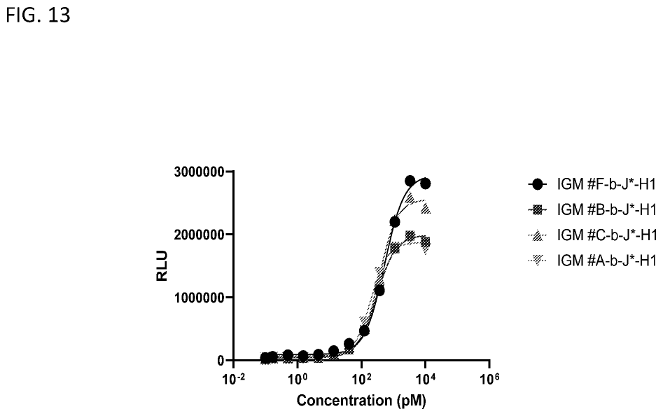

The binding of a subset of the additional anti-CD123×CD3 IgM antibodies to CD123 at different protein concentrations were measured in ELISA assays as described in Example 2. The results are shown in FIG. 13. The data was then analyzed with GraphPad Prism to determine the EC₅₀ and the results are shown in Table 3.

TABLE 3
Antibody Binding IC₅₀

| Antibody | EC₅₀ (pM) |
|---|---|
| IGM #F-b-J*-H1 | 529.7 |
| IGM #B-b-J*-H1 | 298.0 |
| IGM #C-b-J*-H1 | 354.4 |
| IGM #A-b-J*-H1 | 198.0 |

### Example 9: MV4-11-IgM and IgG Binding Assay

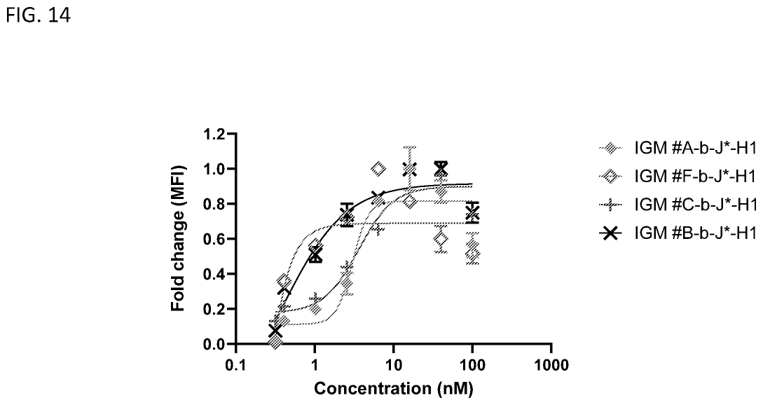

To assess the ability of a subset of the additional anti-CD123×CD3 IgM antibodies to bind CD123 on MV4-11 cells expressing the CD123 protein, a binding assay was performed of various concentrations of antibody by the method described in Example 3 under the heading “AML cell lines IgM and IgG Binding Assay.” The results are shown in FIG. 14.

### Example 10: T Cell-Directed AML Cell Killing-Different CD123 Binding Domains

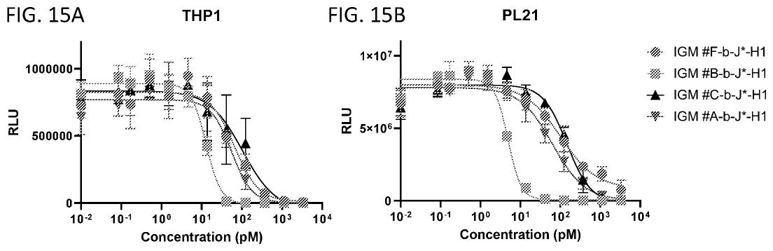

In order to demonstrate that bispecific CD123×CD3 IgM binding molecules comprising different CD123 binding domains can kill target cells in the presence of human T-cells, co-culture experiments were performed with 4 exemplary anti-CD123×CD3 IgM binding molecules, each comprising different CD123 binding domains (IGM #F-b-J*-H1, IGM #B-b-J*-H1, IGM #C-b-J*-H1, and IGM #A-b-J*-H1). 5×10³ Tumor cells MV4-11, THP-1, and PL-21 (all expressing firefly luciferase) were co-cultured with T cells (either strong donor or weak donor T cells) at 7:1 Effector to target (E:T) ratios in the presence of serial dilutions of antibody in 100 μL total volume of AIM-V media supplemented with 3% heat-inactivated fetal bovine serum (FBS) per well on a 96 round bottom tissue culture plate. After 72 or 96 hours of incubation at 37° C. in a 5% CO₂ incubator, 50 μl of luciferase substrate e.g., ONE-Glo EX Luciferase Assay System, Promega was added to the wells. The plates were shaken briefly to mix the reagents, and luciferase luminescent signal was measured on an EnVision plate reader (Perkin-Elmer). The data was then analyzed with GraphPad Prism to determine the EC₅₀. The results for co-cultures of strong donor T cells and THP1 or PL21 are shown in FIGS. 15A and 15B, respectively and EC₅₀ values for all co-cultures are shown in Table 4.

TABLE 4
T Cell-Directed Killing EC₅₀ (pM)

| | Strong Donor T cells | | | Weak Donor T cells | | |
|---|---|---|---|---|---|---|
| Antibody | MV411 | THP-1 | PL21 | MV411 | THP-1 | PL21 |
| IGM #F-b-J*-H1 | 273.2 | 66.3 | 101.7 | 1082 | NA | NA |
| IGM #B-b-J*-H1 | 41.2 | 12.9 | 4.9 | 35.3 | 46.2 | 226.8 |
| IGM #C-b-J*-H1 | 0.1 | 103.7 | 146.7 | 527.5 | 989.8 | NA |
| IGM #A-b-J*-H1 | 328.6 | 45.6 | 63.5 | 728.8 | 329.2 | NA |

### Example 11: T Cell-Directed AML Cell Killing-Different Modified J-Chains

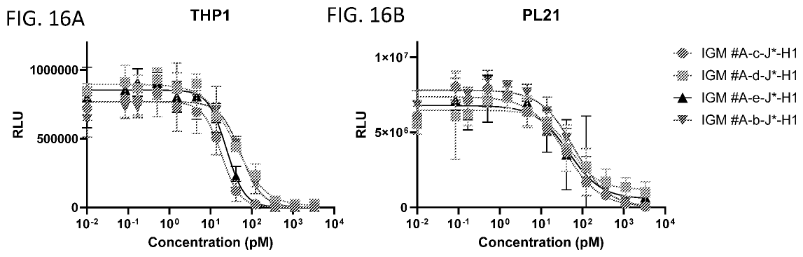

In order to demonstrate that bispecific CD123×CD3 IgM binding molecules comprising different CD3 binding domains can kill target cells in the presence of human T-cells, co-culture experiments were performed with 4 exemplary anti-CD123×CD3 IgM binding molecules, each comprising different CD123 binding domains (IGM #A-c-J*-H1, IGM #A-d-J*-H1, IGM #A-e-J*-H1, and IGM #A-b-J*-H1). 5×10³ Tumor cells MV4-11, THP-1, and PL-21 (all expressing firefly luciferase) were co-cultured with T cells (either strong donor or weak donor T cells) at 7:1 Effector to target (E:T) ratios in the presence of serial dilutions of antibody in 100 μL total volume of AIM-V media supplemented with 3% heat-inactivated fetal bovine serum (FBS) per well on a 96 round bottom tissue culture plate. After 72 or 96 hours of incubation at 37° C. in a 5% CO₂ incubator. 50 μl of luciferase substrate e.g., ONE-Glo EX Luciferase Assay System, Promega was added to the wells. The plates were shaken briefly to mix the reagents, and luciferase luminescent signal was measured on an EnVision plate reader (Perkin-Elmer). The data was then analyzed with GraphPad Prism to determine the EC₅₀. The results for co-cultures of strong donor T cells and THP1 or PL21 are shown in FIGS. 16A and 16B, respectively and EC₅₀ values for all co-cultures are shown in Table 5.

TABLE 5
T Cell-Directed Killing EC₅₀ (pM)

| | Strong Donor T cells | | | Weak Donor T cells | | |
|---|---|---|---|---|---|---|
| Antibody | MV411 | THP-1 | PL21 | MV411 | THP-1 | PL21 |
| IGM #A-c-J*-H1 | 52.2 | 18.5 | 37.9 | 212.5 | 115.5 | N/A |
| IGM #A-d-J*-H1 | 331.1 | 40.3 | 50.8 | 670.5 | 1053 | N/A |
| IGM #A-e-J*-H1 | 110.6 | 24.2 | 46.2 | 360.3 | 454.6 | 94.9 |
| IGM #A-b-J*-H1 | 257.5 | 55.1 | 63.5 | 1010 | 94.91 | N/A |

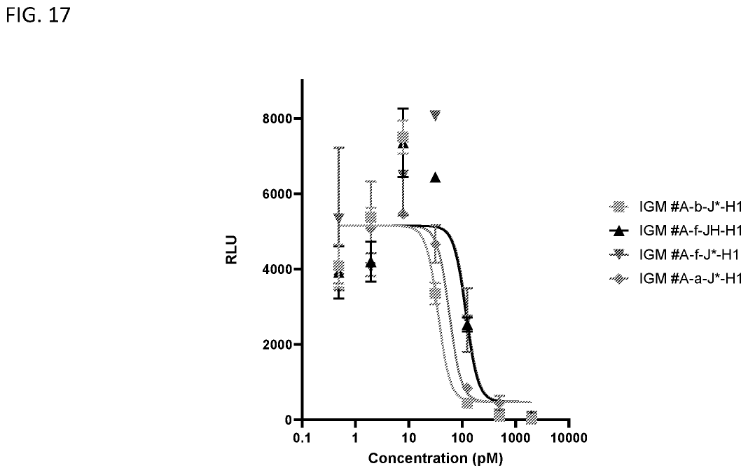

To compare other CD3 binding domains and J* compared to J-HSA, the assay was repeated generally as described above. MV4-11 cells were co-cultured with strong donor cells at an E:T ratio of 3:1 in the presence of IGM #A-b-J*-H1, IGM #A-f-JH-H1, IGM #A-f-J*-H1, or IGM #A-a-J*-H1. The results are shown in FIG. 17.

### Example 12: CD4+ vs CD8+ T Cell-Directed AML Cell Killing

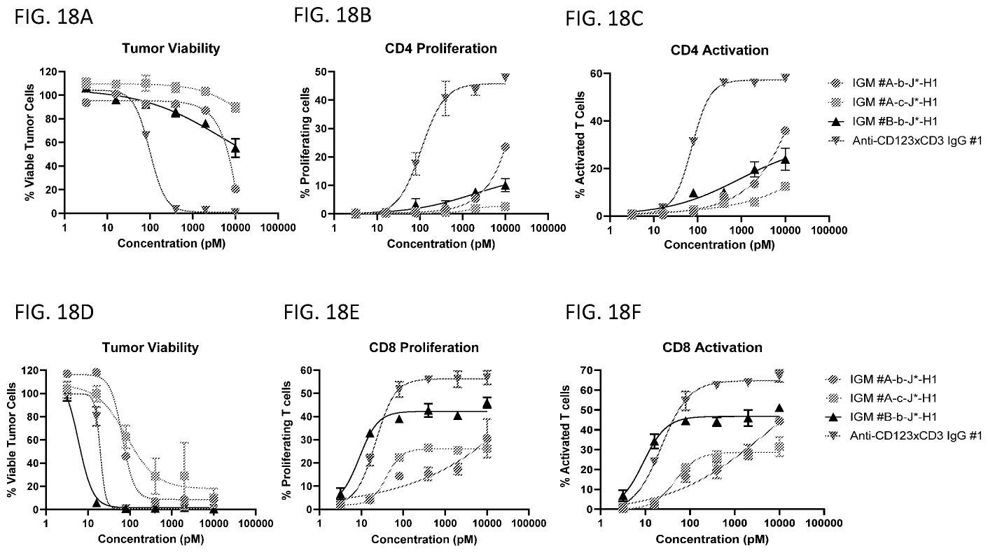

The ability of a subset of the additional Anti-CD123×CD3 IgM antibodies to enhance T cell activation was assessed as described in Example 5. The results are shown in FIGS. 18A-18F. Anti-CD123×CD3 IgM potent tumor mediated cytotoxicity and T cell proliferation with CD8+ T cells but not CD4+ T cells.

### Example 13: Cytokine Release

In order to determine the amount of various cytokines released with exposure bispecific CD123×CD3 IgM binding molecules, co-culture experiments were performed with 3 exemplary anti-CD123×CD3 IgM binding molecules (IGM #B-b-J*-H1, IGM #A-c-J*-H1, or IGM #A-b-J*-H1) or Anti-CD123×CD3 IgG #1. 5×10³ MV4-11 tumor cells expressing firefly luciferase were co-cultured with T cells at 7:1 Effector to target (E:T) ratios in the presence of 50 pM or 1 nM antibody in 100 μL total volume of AIM-V media supplemented with 3% heat-inactivated fetal bovine serum (FBS) per well on a 96 round bottom tissue culture plate. After 72 or 96 hours of incubation at 37° C. in a 5% C02 incubator, 50 μl of supernatant was removed from the co-cultures when 100% of tumor cells had been killed and was frozen at −80° C. until analyzed.

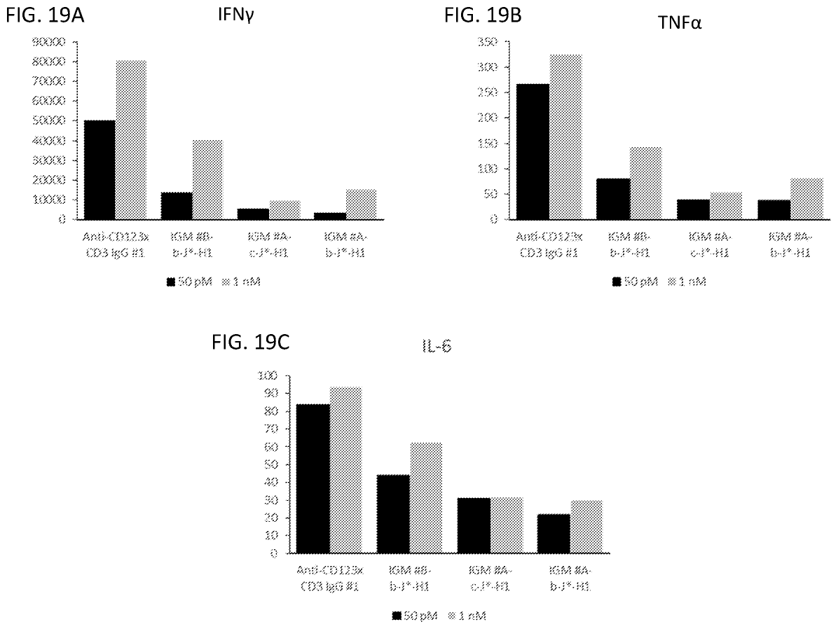

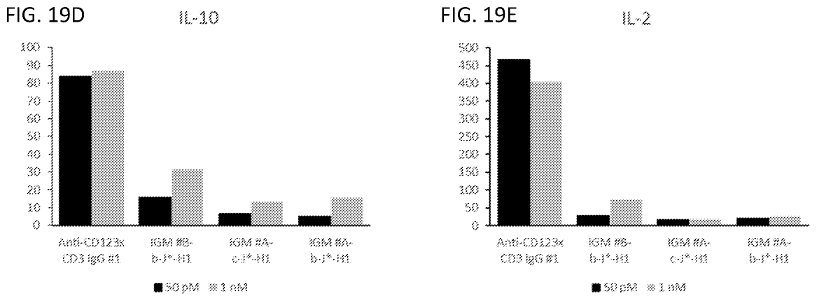

Supernatants were assayed for a panel of cytokines including IFNγ, TNFα, IL-6, IL-10, and IL-2 using V-PLEX Proinflammatory Panel human (MSD, K15049D-2) according to manufacturer's protocol. The results were then analyzed with GraphPad Prism. The results are shown in FIGS. 19A-E and Table 6. The IgM antibodies resulted in less cytokine release at both concentrations of antibodies for all cytokines assayed.

TABLE 6
Released Cytokine Concentration (pg/mL)

| | IFNγ | | TNFa | | IL-6 | | IL-10 | | IL-2 | |
|---|---|---|---|---|---|---|---|---|---|---|
| Conc (pM) | 50 | 1000 | 50 | 1000 | 50 | 1000 | 50 | 1000 | 50 | 1000 |
| Anti-CD123 × CD3 IgG #1 | 49929 | 80387 | 266 | 325 | 84 | 93 | 84 | 87 | 466 | 404 |
| IGM #B-b-J*-H1 | 13342 | 40337 | 79 | 143 | 44 | 62 | 16 | 32 | 29 | 73 |
| IGM #A-c-J*-H1 | 5250 | 9477 | 39 | 54 | 31 | 32 | 7 | 13 | 16 | 17 |
| IGM #A-b-J*-H1 | 3200 | 15240 | 37 | 81 | 22 | 30 | 5 | 16 | 20 | 25 |

TABLE 7
Sequences Presented in the Disclosure

| SEQ | | |
|---|---|---|
| ID | Short Name; Source | Sequence |
| 1 | Precursor Human J | MKNHLLFWGVLAVFIKAVHVKAQEDERIVLVDNKC |
| | Chain | KCARITSRIIRSSEDPNEDIVERNIRIIVPLNNRENISDP |
| | | TSPLRTRFVYHLSDLCKKCDPTEVELDNQIVTATQS |
| | | NICDEDSATETCYTYDRNKCYTAVVPLVYGGETKM |
| | | VETALTPDACYPD |
| 2 | Mature Human J Chain | QEDERIVLVDNKCKCARITSRIIRSSEDPNEDIVERNI |
| | | RIIVPLNNRENISDPTSPLRTRFVYHLSDLCKKCDPTE |
| | | VELDNQIVTATQSNICDEDSATETCYTYDRNKCYTA |
| | | VVPLVYGGETKMVETALTPDACYPD |
| 3 | Mature J* | QEDERIVLVDNKCKCARITSRIIRSSEDPNEDIVERNI |
| | | RIIVPLNNRENISDPTSPLRTRFVYHLSDLCKKCDPTE |
| | | VELDNQIVTATQSNICDEDSATETCATYDRNKCYTA |
| | | VVPLVYGGETKMVETALTPDACYPD |
| 4 | anti CD3 SP34 VH | EVQLVESGGGLVQPKGSLKLSCAASGFTFNTYAMN |
| | e.g., WO2015095392 | WVRQAPGKGLEWVARIRSKYNNYATYYADSVKDR |
| | | FTISRDDSQSILYLQMNNLKTEDTAMYYCVRHGNFG |
| | | NSYVSWFAYWGQGTLVTVSS |
| 5 | SP34 VH CDR1 | TYAMN |
| 6 | SP34 VH CDR2 | RIRSKYNNYATYYADSVKD |
| 7 | SP34 VH CDR3 | HGNFGNSYVSWFAY |
| 8 | anti CD3 SP34 VL | QAVVTQESALTTSPGETVTLTCRSSTGAVTTSNYAN |
| | e.g., WO2015095392 | WVQEKPDHLFTGLIGGTNKRAPGVPARFSGSLIGDK |
| | | AALTITGAQTEDEAIYFCALWYSNLWVFGGGTKLT |
| | | VL |
| 9 | SP34 VL CDR1 | RSSTGAVTTSNYAN |
| 10 | SP34 VL CDR2 | GTNKRAP |
| 11 | SP34 VL CDR3 | ALWYSNLWV |
| 12 | SJ* | MGWSYIILFLVATATGVHSEVQLVESGGGLVQPKGS |
| | | LKLSCAASGFTFNTYAMNWVRQAPGKGLEWVARIR |
| | | SKYNNYATYYADSVKDRFTISRDDSQSILYLQMNNL |
| | | KTEDTAMYYCVRHGNFGNSYVSWFAYWGQGTLVT |
| | | VSSGGGGSGGGGSGGGGSQAVVTQESALTTSPGET |
| | | VTLTCRSSTGAVTTSNYANWVQEKPDHLFTGLIGGT |
| | | NKRAPGVPARFSGSLIGDKAALTITGAQTEDEAIYFC |
| | | ALWYSNLWVFGGGTKLTVLGGGGSGGGGSGGGGS |
| | | QEDERIVLVDNKCKCARITSRIIRSSEDPNEDIVERNI |
| | | RIIVPLNNRENISDPTSPLRTRFVYHLSDLCKKCDPTE |
| | | VELDNQIVTATQSNICDEDSATETCATYDRNKCYTA |
| | | VVPLVYGGETKMVETALTPDACYPD |
| 13 | Visilizumab VH | QVQLVQSGAEVKKPGASVKVSCKASGYTFISYTMH |
| | US5834597A | WVRQAPGQGLEWMGYINPRSGYTHYNQKLKDKAT |
| | | LTADKSASTAYMELSSLRSEDTAVYYCARSAYYDY |
| | | DGFAYWGQGTLVTVSS |
| 14 | Visilizumab VL | DIQMTQSPSSLSASVGDRVTITCSASSSVSYMNWYQ |
| | US5834597A | QKPGKAPKRLIYDTSKLASGVPSRFSGSGSGTDFTLT |
| | | ISSLQPEDFATYYCQQWSSNPPTFGGGTKLEIK |
| 15 | precursor modified J- | MGWSYIILFLVATATGVHSQVQLVQSGAEVKKPGA |
| | chain sequence for | SVKVSCKASGYTFISYTMHWVRQAPGQGLEWMGYI |
| | V15J* | NPRSGYTHYNQKLKDKATLTADKSASTAYMELSSL |
| | | RSEDTAVYYCARSAYYDYDGFAYWGQGTLVTVSS |
| | | GGGGSGGGGSGGGGSDIQMTQSPSSLSASVGDRVTI |
| | | TCSASSSVSYMNWYQQKPGKAPKRLIYDTSKLASG |
| | | VPSRFSGSGSGTDFTLTISSLQPEDFATYYCQQWSSN |
| | | PPTFGGGTKLEIKGGGGSGGGGSGGGGSQEDERIVL |
| | | VDNKCKCARITSRIIRSSEDPNEDIVERNIRIIVPLNNR |
| | | ENISDPTSPLRTRFVYHLSDLCKKCDPTEVELDNQIV |
| | | TATQSNICDEDSATETCATYDRNKCYTAVVPLVYG |
| | | GETKMVETALTPDACYPD |
| 16 | Precursor modified J- | MKNHLLFWGVLAVFIKAVHVKAQEDERIVLVDNKC |
| | chain sequence for | KCARITSRIIRSSEDPNEDIVERNIRIIVPLNNRENISDP |
| | J*15V | TSPLRTRFVYHLSDLCKKCDPTEVELDNQIVTATQS |
| | | NICDEDSATETCATYDRNKCYTAVVPLVYGGETKM |
| | | VETALTPDACYPDGGGGSGGGGSGGGGSQVQLVQS |
| | | GAEVKKPGASVKVSCKASGYTFISYTMHWVRQAPG |
| | | QGLEWMGYINPRSGYTHYNQKLKDKATLTADKSAS |
| | | TAYMELSSLRSEDTAVYYCARSAYYDYDGFAYWG |
| | | QGTLVTVSSGGGGSGGGGSGGGGSDIQMTQSPSSLS |
| | | ASVGDRVTITCSASSSVSYMNWYQQKPGKAPKRLIY |
| | | DTSKLASGVPSRFSGSGSGTDFTLTISSLQPEDFATYY |
| | | CQQWSSNPPTFGGGTKLEIK |
| 17 | Five Linker | GGGGS |
| 18 | Ten Linker | GGGGSGGGGS |
| 19 | Fifteen Linker | GGGGSGGGGSGGGGS |
| 20 | Twenty Linker | GGGGSGGGGSGGGGSGGGGS |
| 21 | Twenty-five Linker | GGGGSGGGGSGGGGSGGGGSGGGGS |
| 22 | Human IgM Constant | GSASAPTLFPLVSCENSPSDTSSVAVGCLAQDFLPDSI |
| | region IMGT allele | TFSWKYKNNSDISSTRGFPSVLRGGKYAATSQVLLP |
| | IGHM*03 | SKDVMQGTDEHVVCKVQHPNGNKEKNVPLPVIAEL |
| | Ig mu chain C region- | PPKVSVFVPPRDGFFGNPRKSKLICQATGFSPRQIQV |
| | human [Homo sapiens] | SWLREGKQVGSGVTTDQVQAEAKESGPTTYKVTST |
| | Sequence ID: | LTIKESDWLSQSMFTCRVDHRGLTFQQNASSMCVPD |
| | pir\|S37768\|Length: 453 | QDTAIRVFAIPPSFASIFLTKSTKLTCLVTDLTTYDSV |
| | Note that sometimes | TISWTRQNGEAVKTHTNISESHPNATFSAVGEASICE |
| | S191 can be G, see | DDWNSGERFTCTVTHTDLPSPLKQTISRPKGVALHR |
| | Sequence ID: P01871.4 | PDVYLLPPAREQLNLRESATITCLVTGFSPADVFVQ |
| | | WMQRGQPLSPEKYVTSAPMPEPQAPGRYFAHSILTV |
| | | SEEEWNTGETYTCVVAHEALPNRVTERTVDKSTGK |
| | | PTLYNVSLVMSDTAGTCY |
| 23 | Human IgM Constant | GSASAPTLFPLVSCENSPSDTSSVAVGCLAQDFLPDSI |
| | region IMGT allele | TFSWKYKNNSDISSTRGFPSVLRGGKYAATSQVLLP |
| | IGHM*04; There are | SKDVMQGTDEHVVCKVQHPNGNKEKNVPLPVIAEL |
| | several alleles. The | PPKVSVFVPPRDGFFGNPRKSKLICQATGFSPRQIQV |
| | sequence shown is that | SWLREGKQVGSGVTTDQVQAEAKESGPTTYKVTST |
| | of IMGT allele | LTIKESDWLGQSMFTCRVDHRGLTFQQNASSMCVP |
| | IGHM*04. | DQDTAIRVFAIPPSFASIFLTKSTKLTCLVTDLTTYDS |
| | | VTISWTRQNGEAVKTHTNISESHPNATFSAVGEASIC |
| | | EDDWNSGERFTCTVTHTDLPSPLKQTISRPKGVALH |
| | | RPDVYLLPPAREQLNLRESATITCLVTGFSPADVFVQ |
| | | WMQRGQPLSPEKYVTSAPMPEPQAPGRYFAHSILTV |
| | | SEEEWNTGETYTCVVAHEALPNRVTERTVDKSTGK |
| | | PTLYNVSLVMSDTAGTCY |
| 24 | Human IgA1 Constant | ASPTSPKVFPLSLCSTQPDGNVVIACLVQGFFPQEPLS |
| | Region | VTWSESGQGVTARNFPPSQDASGDLYTTSSQLTLPA |
| | | TQCLAGKSVTCHVKHYTNPSQDVTVPCPVPSTPPTP |
| | | SPSTPPTPSPSCCHPRLSLHRPALEDLLLGSEANLTCT |
| | | LTGLRDASGVTFTWTPSSGKSAVQGPPERDLCGCYS |
| | | VSSVLPGCAEPWNHGKTFTCTAAYPESKTPLTATLS |
| | | KSGNTFRPEVHLLPPPSEELALNELVTLTCLARGFSP |
| | | KDVLVRWLQGSQELPREKYLTWASRQEPSQGTTTF |
| | | AVTSILRVAAEDWKKGDTFSCMVGHEALPLAFTQK |
| | | TIDRLAGKPTHVNVSVVMAEVDGTCY |
| 25 | Human IgA2 Constant | ASPTSPKVFPLSLDSTPQDGNVVVACLVQGFFPQEPL |
| | Region | SVTWSESGQNVTARNFPPSQDASGDLYTTSSQLTLP |
| | | ATQCPDGKSVTCHVKHYTNPSQDVTVPCPVPPPPPC |
| | | CHPRLSLHRPALEDLLLGSEANLTCTLTGLRDASGA |
| | | TFTWTPSSGKSAVQGPPERDLCGCYSVSSVLPGCAQ |
| | | PWNHGETFTCTAAHPELKTPLTANITKSGNTFRPEV |
| | | HLLPPPSEELALNELVTLTCLARGFSPKDVLVRWLQ |
| | | GSQELPREKYLTWASRQEPSQGTTTFAVTSILRVAA |
| | | EDWKKGDTFSCMVGHEALPLAFTQKTIDRLAGKPT |
| | | HVNVSVVMAEVDGTCY |
| 26 | Human Secretory | MLLFVLTCLLAVFPAISTKSPIFGPEEVNSVEGNSVSI |
| | Component Precursor | TCYYPPTSVNRHTRKYWCRQGARGGCITLISSEGYV |
| | | SSKYAGRANLTNFPENGTFVVNIAQLSQDDSGRYKC |
| | | GLGINSRGLSFDVSLEVSQGPGLLNDTKVYTVDLGR |
| | | TVTINCPFKTENAQKRKSLYKQIGLYPVLVIDSSGYV |
| | | NPNYTGRIRLDIQGTGQLLFSVVINQLRLSDAGQYLC |
| | | QAGDDSNSNKKNADLQVLKPEPELVYEDLRGSVTF |
| | | HCALGPEVANVAKFLCRQSSGENCDVVVNTLGKRA |
| | | PAFEGRILLNPQDKDGSFSVVITGLRKEDAGRYLCG |
| | | AHSDGQLQEGSPIQAWQLFVNEESTIPRSPTVVKGV |
| | | AGGSVAVLCPYNRKESKSIKYWCLWEGAQNGRCPL |
| | | LVDSEGWVKAQYEGRLSLLEEPGNGTFTVILNQLTS |
| | | RDAGFYWCLTNGDTLWRTTVEIKIIEGEPNLKVPGN |
| | | VTAVLGETLKVPCHFPCKFSSYEKYWCKWNNTGCQ |
| | | ALPSQDEGPSKAFVNCDENSRLVSLTLNLVTRADEG |
| | | WYWCGVKQGHFYGETAAVYVAVEERKAAGSRDV |
| | | SLAKADAAPDEKVLDSGFREIENKAIQDPRLFAEEK |
| | | AVADTRDQADGSRASVDSGSSEEQGGSSRALVSTLV |
| | | PLGLVLAVGAVAVGVARARHRKNVDRVSIRSYRTD |
| | | ISMSDFENSREFGANDNMGASSITQETSLGGKEEFVA |
| | | TTESTTETKEPKKAKRSSKEEAEMAYKDFLLQSSTV |
| | | AAEAQDGPQEA |
| 27 | human secretory | KSPIFGPEEVNSVEGNSVSITCYYPPTSVNRHTRKYW |
| | component mature | CRQGARGGCITLISSEGYVSSKYAGRANLTNFPENG |
| | | TFVVNIAQLSQDDSGRYKCGLGINSRGLSFDVSLEVS |
| | | QGPGLLNDTKVYTVDLGRTVTINCPFKTENAQKRKS |
| | | LYKQIGLYPVLVIDSSGYVNPNYTGRIRLDIQGTGQL |
| | | LFSVVINQLRLSDAGQYLCQAGDDSNSNKKNADLQ |
| | | VLKPEPELVYEDLRGSVTFHCALGPEVANVAKFLCR |
| | | QSSGENCDVVVNTLGKRAPAFEGRILLNPQDKDGSF |
| | | SVVITGLRKEDAGRYLCGAHSDGQLQEGSPIQAWQL |
| | | FVNEESTIPRSPTVVKGVAGGSVAVLCPYNRKESKSI |
| | | KYWCLWEGAQNGRCPLLVDSEGWVKAQYEGRLSL |
| | | LEEPGNGTFTVILNQLTSRDAGFYWCLTNGDTLWRT |
| | | TVEIKIIEGEPNLKVPGNVTAVLGETLKVPCHFPCKF |
| | | SSYEKYWCKWNNTGCQALPSQDEGPSKAFVNCDEN |
| | | SRLVSLTLNLVTRADEGWYWCGVKQGHFYGETAA |
| | | VYVAVEERKAAGSRDVSLAKADAAPDEKVLDSGFR |
| | | EIENKAIQDPR |
| 28 | human CD123 isoform 1 | MVLLWLTLLLIALPCLLQTKEDPNPPITNLRMKAKA |
| | precursor NCBI | QQLTWDLNRNVTDIECVKDADYSMPAVNNSYCQF |
| | Reference Sequence: | GAISLCEVTNYTVRVANPPFSTWILFPENSGKPWAG |
| | NP_002174.1 | AENLTCWIHDVDFLSCSWAVGPGAPADVQYDLYLN |
| | | VANRRQQYECLHYKTDAQGTRIGCRFDDISRLSSGS |
| | | QSSHILVRGRSAAFGIPCTDKFVVFSQIEILTPPNMTA |
| | | KCNKTHSFMHWKMRSHFNRKFRYELQIQKRMQPVI |
| | | TEQVRDRTSFQLLNPGTYTVQIRARERVYEFLSAWS |
| | | TPQRFECDQEEGANTRAWRTSLLIALGTLLALVCVF |
| | | VICRRYLVMQRLFPRIPHMKDPIGDSFQNDKLVVWE |
| | | AGKAGLEECLVTEVQVVQKT |
| 29 | human CD123 isoform 2 | MVLLWLTLLLIALPCLLQTKEGGKPWAGAENLTCW |
| | precursor NCBI | IHDVDFLSCSWAVGPGAPADVQYDLYLNVANRRQQ |
| | Reference Sequence: | YECLHYKTDAQGTRIGCRFDDISRLSSGSQSSHILVR |
| | NP_001254642.1 | GRSAAFGIPCTDKFVVFSQIEILTPPNMTAKCNKTHS |
| | | FMHWKMRSHFNRKFRYELQIQKRMQPVITEQVRDR |
| | | TSFQLLNPGTYTVQIRARERVYEFLSAWSTPQRFECD |
| | | QEEGANTRAWRTSLLIALGTLLALVCVFVICRRYLV |
| | | MQRLFPRIPHMKDPIGDSFQNDKLVVWEAGKAGLE |
| | | ECLVTEVQVVQKT |
| 30 | Cyno CD123 GenBank: | MTLLWLTLLLVATPCLLRTKEDPNAPIRNLRMKEKA |
| | EHH61867.1 | QQLMWDLNRNVTDVECIKGTDYSMPAMNNSYCQF |
| | | GAISLCEVTNYTVRVASPPFSTWILFPENSGTPRAGA |
| | | ENLTCWVHDVDFLSCSWVVGPAAPADVQYDLYLN |
| | | NPNSHEQYRCLHYKTDARGTQIGCRFDDIAPLSRGS |
| | | QSSHILVRGRSAAVSIPCTDKFVFFSQIERLTPPNMTG |
| | | ECNETHSFMHWKMKSHFNRKFRYELRIQKRMQPVR |
| | | TEQVRDTTSFQLPNPGTYTVQIRARETVYEFLSAWS |
| | | TPQRFECDQEEGASSRAWRTSLLIALGTLLALLCVFL |
| | | ICRRYLVMQRLFPRIPHMKDPIGDTFQQDKLVVWEA |
| | | GKAGLEECLVSEVQVVEKT |
| 31 | Mouse CD123 NCBI | MAANLWLILGLLASHSSDLAAVREAPPTAVTTPIQN |
| | Reference Sequence: | LHIDPAHYTLSWDPAPGADITTGAFCRKGRDIFVWA |
| | NP_032395.1 | DPGLARCSFQSLSLCHVTNFTVFLGKDRAVAGSIQFP |
| | | PDDDGDHEAAAQDLRCWVHEGQLSCQWERGPKAT |
| | | GDVHYRMFWRDVRLGPAHNRECPHYHSLDVNTAG |
| | | PAPHGGHEGCTLDLDTVLGSTPNSPDLVPQVTITVN |
| | | GSGRAGPVPCMDNTVDLQRAEVLAPPTLTVECNGS |
| | | EAHARWVARNRFHHGLLGYTLQVNQSSRSEPQEYN |
| | | VSIPHFWVPNAGAISFRVKSRSEVYPRKLSSWSEAW |
| | | GLVCPPEVMPVKTALVTSVATVLGAGLVAAGLLLW |
| | | WRKSLLYRLCPPIPRLRLPLAGEMVVWEPALEDCEV |
| | | TPVTDA |
| 32 | anti-CD123 #1 VH | QVQLQQSGAEVKKPGASVKVSCKASGYTFTDYYM |
| | US 9856327 B2 | KWVKQSHGKSLEWMGDIIPSNGATFYNQKFKGKAT |
| | | LTVDRSTSTAYMELSSLRSEDTAVYYCARSHLLRAS |
| | | WFAYWGQGTLVTVSS |
| 33 | anti-CD123 #1 VL | DFVMTQSPDSLAVSLGERATINCKSSQSLLNTGNQK |
| | US 9856327 B2 | NYLTWYQQKPGQPPKLLIYWASTRESGVPDRFTGSG |
| | | SGTDFTLTISSLQAEDVAVYYCQNDYSYPYTFGGGT |
| | | KLEIK |
| 34 | anti-CD123_IgG_#1 | MGWSYIILFLVATATGVHSQVQLQQSGAEVKKPGA |
| | US 9856327 B2 | SVKVSCKASGYTFTDYYMKWVKQSHGKSLEWMGD |
| | | IIPSNGATFYNQKFKGKATLTVDRSTSTAYMELSSLR |
| | | SEDTAVYYCARSHLLRASWFAYWGQGTLVTVSSAS |
| | | TKGPSVFPLAPSSKSTSGGTAALGCLVKDYFPEPVTV |
| | | SWNSGALTSGVHTFPAVLQSSGLYSLSSVVTVPSSSL |
| | | GTQTYICNVNHKPSNTKVDKRVEPKSCDKTHTCPPC |
| | | PAPELLGGPSVFLFPPKPKDTLMISRTPEVTCVVVDV |
| | | SHEDPEVKFNWYVDGVEVHNAKTKPREEQYNSTYR |
| | | VVSVLTVLHQDWLNGKEYKCKVSNKALPAPIEKTIS |
| | | KAKGQPREPQVYTLPPSRDELTKNQVSLTCLVKGFY |
| | | PSDIAVEWESNGQPENNYKTTPPVLDSDGSFFLYSKL |
| | | TVDKSRWQQGNVFSCSVMHEALHNHYTQKSLSLSP |
| | | GK |
| 35 | anti-CD123_IgM_#1 | MGWSYIILFLVATATGVHSQVQLQQSGAEVKKPGA |
| | | SVKVSCKASGYTFTDYYMKWVKQSHGKSLEWMGD |
| | | IIPSNGATFYNQKFKGKATLTVDRSTSTAYMELSSLR |
| | | SEDTAVYYCARSHLLRASWFAYWGQGTLVTVSSGS |
| | | ASAPTLFPLVSCENSPSDTSSVAVGCLAQDFLPDSITF |
| | | SWKYKNNSDISSTRGFPSVLRGGKYAATSQVLLPSK |
| | | DVMQGTDEHVVCKVQHPNGNKEKNVPLPVIAELPP |
| | | KVSVFVPPRDGFFGNPRKSKLICQATGFSPRQIQVSW |
| | | LREGKQVGSGVTTDQVQAEAKESGPTTYKVTSTLTI |
| | | KESDWLSQSMFTCRVDHRGLTFQQNASSMCVPDQD |
| | | TAIRVFAIPPSFASIFLTKSTKLTCLVTDLTTYDSVTIS |
| | | WTRQNGEAVKTHTNISESHPNATFSAVGEASICEDD |
| | | WNSGERFTCTVTHTDLPSPLKQTISRPKGVALHRPD |
| | | VYLLPPAREQLNLRESATITCLVTGFSPADVFVQWM |
| | | QRGQPLSPEKYVTSAPMPEPQAPGRYFAHSILTVSEE |
| | | EWNTGETYTCVVAHEALPNRVTERTVDKSTGKPTL |
| | | YNVSLVMSDTAGTCY |
| 36 | anti-CD123 kappa #1 | MRVPAQLLGLLLLWLRGARCDFVMTQSPDSLAVSL |
| | | GERATINCKSSQSLLNTGNQKNYLTWYQQKPGQPP |
| | | KLLIYWASTRESGVPDRFTGSGSGTDFTLTISSLQAE |
| | | DVAVYYCQNDYSYPYTFGGGTKLEIKRTVAAPSVFI |
| | | FPPSDEQLKSGTASVVCLLNNFYPREAKVQWKVDN |
| | | ALQSGNSQESVTEQDSKDSTYSLSSTLTLSKADYEK |
| | | HKVYACEVTHQGLSSPVTKSFNRGEC |
| 37 | Anti-CD123 #2 VH | QVQLVQSGAEVKKPGASVKVSCKASGYTFTSYWM |
| | US20160068601A1 | NWVRQAPGQGLEWMGRIDPYDSETHYNQKFKDRV |
| | | TMTVDKSTSTAYMELSSLRSEDTAVYYCARGNWDD |
| | | YWGQGTTVTVSS |
| 38 | Anti-CD123 #2 VL | EVVLTQSPATLSLSPGERATLSCRASKSISKDLAWYQ |
| | US20160068601A1 | QKPGQAPRLLIYSGSTLQSGIPARFSGSGSGTDFTLTI |
| | | SSLEPEDFAVYYCQQHNKYPYTFGGGTKVEIK |
| 39 | Anti-CD123_IgG_#2 | MGWSYIILFLVATATGVHSQVQLVQSGAEVKKPGA |
| | US20160068601A1 | SVKVSCKASGYTFTSYWMNWVRQAPGQGLEWMG |
| | | RIDPYDSETHYNQKFKDRVTMTVDKSTSTAYMELSS |
| | | LRSEDTAVYYCARGNWDDYWGQGTTVTVSSASTK |
| | | GPSVFPLAPSSKSTSGGTAALGCLVKDYFPEPVTVS |
| | | WNSGALTSGVHTFPAVLQSSGLYSLSSVVTVPSSSL |
| | | GTQTYICNVNHKPSNTKVDKRVEPKSCDKTHTCPPC |
| | | PAPELLGGPSVFLFPPKPKDTLMISRTPEVTCVVVDV |
| | | SHEDPEVKFNWYVDGVEVHNAKTKPREEQYNSTYR |
| | | VVSVLTVLHQDWLNGKEYKCKVSNKALPAPIEKTIS |
| | | KAKGQPREPQVYTLPPSRDELTKNQVSLTCLVKGFY |
| | | PSDIAVEWESNGQPENNYKTTPPVLDSDGSFFLYSKL |
| | | TVDKSRWQQGNVFSCSVMHEALHNHYTQKSLSLSP |
| | | GK |
| 40 | Anti-CD123_IgM_#2 | MGWSYIILFLVATATGVHSQVQLVQSGAEVKKPGA |
| | | SVKVSCKASGYTFTSYWMNWVRQAPGQGLEWMG |
| | | RIDPYDSETHYNQKFKDRVTMTVDKSTSTAYMELSS |
| | | LRSEDTAVYYCARGNWDDYWGQGTTVTVSSGSAS |
| | | APTLFPLVSCENSPSDTSSVAVGCLAQDFLPDSITFS |
| | | WKYKNNSDISSTRGFPSVLRGGKYAATSQVLLPSKD |
| | | VMQGTDEHVVCKVQHPNGNKEKNVPLPVIAELPPK |
| | | VSVFVPPRDGFFGNPRKSKLICQATGFSPRQIQVSWL |
| | | REGKQVGSGVTTDQVQAEAKESGPTTYKVTSTLTIK |
| | | ESDWLSQSMFTCRVDHRGLTFQQNASSMCVPDQDT |
| | | AIRVFAIPPSFASIFLTKSTKLTCLVTDLTTYDSVTISW |
| | | TRQNGEAVKTHTNISESHPNATFSAVGEASICEDDW |
| | | NSGERFTCTVTHTDLPSPLKQTISRPKGVALHRPDVY |
| | | LLPPAREQLNLRESATITCLVTGFSPADVFVQWMQR |
| | | GQPLSPEKYVTSAPMPEPQAPGRYFAHSILTVSEEEW |
| | | NTGETYTCVVAHEALPNRVTERTVDKSTGKPTLYN |
| | | VSLVMSDTAGTCY |
| 41 | Anti-CD123_kappa_#2 | MRVPAQLLGLLLLWLRGARCEVVLTQSPATLSLSPG |
| | US20160068601A1 | ERATLSCRASKSISKDLAWYQQKPGQAPRLLIYSGST |
| | | LQSGIPARFSGSGSGTDFTLTISSLEPEDFAVYYCQQH |
| | | NKYPYTFGGGTKVEIKRTVAAPSVFIFPPSDEQLKSG |
| | | TASVVCLLNNFYPREAKVQWKVDNALQSGNSQESV |
| | | TEQDSKDSTYSLSSTLTLSKADYEKHKVYACEVTHQ |
| | | GLSSPVTKSFNRGEC |
| 104 | Anti-CD123xCD3 IgG | QVQLQQSGAEVKKPGASVKVSCKASGYTFTDYYM |
| | #1, first heavy chain | KWVKQSHGKSLEWMGDIIPSNGATFYNQKFKGKAT |
| | WO 2017/210443 A1 | LTVDRSTSTAYMELSSLRSEDTAVYYCARSHLLRAS |
| | | WFAYWGQGTLVTVSSASTKGPSVFPLAPSSKSTSGG |
| | | TAALGCLVKDYFPEPVTVSWNSGALTSGVHTFPAVL |
| | | QSSGLYSLSSVVTVPSSSLGTQTYICNVNHKPSDTKV |
| | | DKKVEPKSCDKTHTCPPCPAPPVAGPSVFLFPPKPKD |
| | | TLMISRTPEVTCVVVDVKHEDPEVKFNWYVDGVEV |
| | | HNAKTKPREEEYNSTYRVVSVLTVLHQDWLNGKEY |
| | | KCKVSNKALPAPIEKTISKAKGQPREPQVYTLPPSRE |
| | | EMTKNQVSLTCDVSGFYPSDIAVEWESDGQPENNY |
| | | KTTPPVLDSDGSFFLYSKLTVDKSRWEQGDVFSCSV |
| | | MHEALHNHYTQKSLSLSPGK |
| 105 | Anti-CD123xCD3 IgG | DFVMTQSPDSLAVSLGERATINCKSSQSLLNTGNQK |
| | #1, light chain | NYLTWYQQKPGQPPKLLIYWASTRESGVPDRFTGSG |
| | WO 2017/210443 A1 | SGTDFTLTISSLQAEDVAVYYCQNDYSYPYTFGGGT |
| | | KLEIKRTVAAPSVFIFPPSDEQLKSGTASVVCLLNNF |
| | | YPREAKVQWKVDNALQSGNSQESVTEQDSKDSTYS |
| | | LSSTLTLSKADYEKHKVYACEVTHQGLSSPVTKSFN |
| | | RGEC |
| 106 | Anti-CD123xCD3 IgG | EVQLVESGGGLVQPGGSLRLSCAASGFTFSTYAMN |
| | #1, second heavy chain | WVRQAPGKGLEWVGRIRSKYNNYATYYADSVKGR |
| | WO 2017/210443 A1 | FTISRDDSKNTLYLQMNSLRAEDTAVYYCVRHGNF |
| | | GDSYVSWFAYWGQGTLVTVSSGKPGSGKPGSGKPG |
| | | SGKPGSQAVVTQEPSLTVSPGGTVTLTCGSSTGAVT |
| | | TSNYANWVQQKPGKSPRGLIGGTNKRAPGVPARFS |
| | | GSLLGGKAALTISGAQPEDEADYYCALWYSNHWVF |
| | | GGGTKLTVLEPKSSDKTHTCPPCPAPPVAGPSVFLFP |
| | | PKPKDTLMISRTPEVTCVVVDVKHEDPEVKFNWYV |
| | | DGVEVHNAKTKPREEQYNSTYRVVSVLTVLHQDW |
| | | LNGKEYKCKVSNKALPAPIEKTISKAKGQPREPQVY |
| | | TLPPSREQMTKNQVKLTCLVKGFYPSDIAVEWESNG |
| | | QPENNYKTTPPVLDSDGSFFLYSKLTVDKSRWQQG |
| | | NVFSCSVMHEALHNHYTQKSLSLSPGK |
| 129 | J-HSA | QEDERIVLVDNKCKCARITSRIIRSSEDPNEDIVERNI |
| | | RIIVPLNNRENISDPTSPLRTRFVYHLSDLCKKCDPTE |
| | | VELDNQIVTATQSNICDEDSATETCYTYDRNKCYTA |
| | | VVPLVYGGETKMVETALTPDACYPDGGGGSGGGGS |
| | | GGGGSDAHKSEVAHRFKDLGEENFKALVLIAFAQY |
| | | LQQCPFEDHVKLVNEVTEFAKTCVADESAENCDKS |
| | | LHTLFGDKLCTVATLRETYGEMADCCAKQEPERNE |
| | | CFLQHKDDNPNLPRLVRPEVDVMCTAFHDNEETFL |
| | | KKYLYEIARRHPYFYAPELLFFAKRYKAAFTECCQA |
| | | ADKAACLLPKLDELRDEGKASSAKQRLKCASLQKF |
| | | GERAFKAWAVARLSQRFPKAEFAEVSKLVTDLTKV |
| | | HTECCHGDLLECADDRADLAKYICENQDSISSKLKE |
| | | CCEKPLLEKSHCIAEVENDEMPADLPSLAADFVESK |
| | | DVCKNYAEAKDVFLGMFLYEYARRHPDYSVVLLLR |
| | | LAKTYETTLEKCCAAADPHECYAKVFDEFKPLVEEP |
| | | QNLIKQNCELFKQLGEYKFQNALLVRYTKKVPQVST |
| | | PTLVEVSRNLGKVGSKCCKHPEAKRMPCAEDYLSV |
| | | VLNQLCVLHEKTPVSDRVTKCCTESLVNRRPCFSAL |
| | | EVDETYVPKEFNAETFTFHADICTLSEKERQIKKQTA |
| | | LVELVKHKPKATKEQLKAVMDDFAAFVEKCCKAD |
| | | DKETCFAEEGPKLVAASQAALGL |
| 130 | WO2018208864 | TYAMN |
| 131 | WO2018208864 | DYYMH |
| 132 | WO2018208864 | RIRSKYNNYATYYADSVKD |
| 133 | WO2018208864 | WIDLENANTIYDAKFQG |
| 134 | WO2018208864 | WIDLENANTVYDAKFQG |
| 135 | WO2018208864 | HANFGAGYVSWFAH |
| 136 | WO2018208864 | DAYGRYFYDV |
| 137 | WO2018208864 | DAYGQYFYDV |
| 138 | WO2018208864 | GSSTGAVTTSNYAN |
| 139 | WO2018208864 | KSSQSLLNARTGKNYLA |
| 140 | WO2018208864 | GTDKRAP |
| 141 | WO2018208864 | WASTRES |
| 142 | WO2018208864 | ALWYSNHWV |
| 143 | WO2018208864 | ALWYSDLWV |
| 144 | WO2018208864 | KQSYSRRT |
| 145 | WO2018208864 | KQSYFRRT |
| 146 | WO2018208864 | TQSYFRRT |

TABLE 8
Additional anti-CD123 VH and VL Sequences

| | SEQ | | SEQ | |
|---|---|---|---|---|
| Citation | ID | VH | ID | VL |
| WO2018152547A1 | 42 | EVQLVQSGAEVKKPGESLKISCKGSGYSFTD | 43 | DIVMTQSPDSLAVSLGERATINCESSQSLLNS |
| | | YYMKWARQMPGKGLEWMGDIIPSNGATFY | | GNQKNYLTWYQQKPGQPPKPLIYWASTRESG |
| | | NQKFKGQVTISADKSISTTYLQWSSLKASDT | | VPDRFSGSGSGTDFTLTISSLQAEDVAVYYCQ |
| | | AMYYCARSHLLRASWFAYWGQGTMVTVSS | | NDYSYPYTFGQGTKLEIK |
| WO2018152547A1 | 44 | QVQLQQPGAELVRPGASVKLSCKASGYTFTS | 45 | DVQITQSPSYLAASPGETITINCRASKSISKDL |
| | | YWMNWVKQRPDQGLEWIGRIDPYDSETHY | | AWYQEKPGKTNKLLIYSGSTLQSGIPSRFSGS |
| | | NQKFKDKAILTVDKSSSTAYMQLSSLTSEDS | | GSGTDFTLTISSLEPEDFAMYYCQQHNKYPYT |
| | | AVYYCARGNWDDYWGQGTTLTVSS | | FGGGTKLEIK |
| WO2018152547A1 | 46 | QVQLVQSGAEVKKPGASVKMSCKASGYTFT | 47 | DFVMTQSPDSLAVSLGERATINCKSSQSLLNS |
| | | DYYMKWVKQAPGQGLEWIGDIIPSNGATFY | | GNQKNYLTWYLQKPGQPPKLLIYWASTRESG |
| | | NQKFKGKATLTVDRSISTAYMHLNRLRSDD | | VPDRFSGSGSGTDFTLTISSLQAEDVAVYYCQ |
| | | TAVYYCTRSHLLRASWFAYWGQGTLVTVSS | | NDYSYPYTFGQGTKLEIKR |
| WO2017216028A1 | 48 | QVQLVQSGAEVKKPGSSVKVSCKASGGTFS | 49 | DIVMTQSPDSLAVSLGERATINCESSQSVLNS |
| | | DYYMKWVRQAPGQGLEWMGDIIPSNGATF | | GNQKNYLTWYQQKPGQPPKLLIYWASTRES |
| | | YNQKFKGQVTITADESTSTAYMELSSLRSED | | GVPDRFSGSGSGTDFTLTISSLQAEDVAVYYC |
| | | TAVYYCARSHLLRASWFAYWGQGTLVTVSS | | QNDYSYPYTFGQGTKLEIK |
| WO2017216028A1 | 50 | QVQLVQSGAEVKKPGSSVKVSCKASGGTFS | 51 | DIVMTQSPDSLAVSLGERATINCESSQSVLNS |
| | | DYYMKWVRQAPGQGLEWMGDIIPSNGATF | | GNQKNYLTWYQQKPGQPPKLLIYWASTRES |
| | | YNQKFKGRVTITADESTSTAYMELSSLRSED | | GVPDRFSGSGSGTDFTLTISSLQAEDVAVYYC |
| | | TAVYYCARSHLLRASWFAYWGQGTLVTVSS | | QNDYSYPYTFGQGTKLEIK |
| WO2017216028A1 | 52 | EVQLLESGGGLVQPGGSLRLSCAVSDYSITS | 53 | EIVLTQSPGTLSLSPGERATLSCKSSQSLFFGST |
| | | GYYWNWIRQAPGKKLEWMGYISYDGSNNY | | QKNYLAWYQQKPGQAPRLLIYWASTRESGIP |
| | | NPSLKNGRITISRDTSKNTFYLQMNSLRAEDT | | DRFSGSGSGTDFTLTISRLEPEDFAVYYCQQY |
| | | AVYYCARGEGFYFDSWGQGTLVTVSS | | YNYPWTFGQGTKLEIK |
| WO2017216028A1 | 54 | EVQLLESGGGLVQPGGSLRLSCAVSDYSITS | 55 | EIVLTQSPGTLSLSPGERATLSCRASQSVFFGS |
| | | GYYWNWIRQAPGKKLEWMGYISYDGSNNY | | TQKNYLAWYQQKPGQAPRLLIYWASTRESGI |
| | | NPSLKNGRITISRDTSKNTFYLQMNSLRAEDT | | PDRFSGSGSGTDFTLTISRLEPEDFAVYYCQQ |
| | | AVYYCARGEGFYFDSWGQGTLVTVSS | | YYNYPWTFGQGTKLEIK |
| US20180318437A1 | 56 | QVQLVQSGAEVKKPGASVKVSCKASGYTFT | 57 | DIVMTQSPDSLAVSLGERATINCKSSQSLLNS |
| | | DYYMKWVRQAPGQGLEWMGDIIPSNGATFY | | GNQKNYLTWYQQKPGQPPKLLIYWASTRES |
| | | AQKFQGRVTMTRDTSTSTVYMELSSLRSED | | GVPDRFSGSGSGTDFTLTISSLQAEDVAVYYC |
| | | TAVYYCARSHLLRASWFAYWGQGTLVTVSS | | QNDYSYPYTFGQGTKLEIK |
| US20180318437A1 | 58 | QVQLVQSGAEVKKPGSSVKVSCKASGGTFS | 59 | DIVMTQSPDSLAVSLGERATINCESSQSLLNS |
| | | DYYMKWVRQAPGQGLEWMGDIIPSNGATF | | GNQKNYLTWYQQKPGQPPKLLIYWASTRES |
| | | YAQKFQGRVTITADESTSTAYMELSSLRSED | | GVPDRFSGSGSGTDFTLTISSLQAEDVAVYYC |
| | | TAVYYCARSHLLRASWFAYWGQGTLVTVSS | | QNDYSYPYTFGQGTKLEIK |
| US20180318437A1 | 60 | QVQLVQSGAEVKKPGSSVKVSCKASGGTFS | 61 | DIVMTQSPDSLAVSLGERATINCKSSQSLLNS |
| | | DYYMKWVRQAPGQGLEWMGDIIPSNGATF | | GNQKNYLTWYQQKPGQPPKLLIYWASTRES |
| | | YAQKFQGRVTITADESTSTAYMELSSLRSED | | GVPDRFSGSGSGTDFTLTISSLQAEDVAVYYC |
| | | TAVYYCARSHLLRASWFAYWGQGTLVTVSS | | QNDYSYPYTFGQGTKLEIK |
| US20180318437A1 | 62 | QVQLQESGPGLVKPSQSLSLTCSVTDYSITSG | 63 | DIMMSQSPSSLAVSVGEKFTMTCKSSQSLFFG |
| | | YYWNWIRQFPGNKLEWMGYISYDGSNNYN | | STQKNYLAWYQQKPGQSPKLLIYWASTRESG |
| | | PSLKNRISITRDTSKNQFFLKLSSVTTEDTAT | | VPDRFTGSGSGTDFTLAISSVMPEDLAVYYCQ |
| | | YYCSRGEGFYFDSWGQGTTLTVSS | | QYYNYPWTFGGGTKLEIK |
| US20180169261A1 | 64 | QVQLVQSGAEVKKPGASVKMSCKASGYTFT | 65 | DFVMTQSPDSLAVSLGERATINCKSSQSLLNS |
| | | DYYMKWVKQAPGQGLEWIGDIIPSNGATFY | | GNQKNYLTWYLQKPGQPPKLLIYWASTRESG |
| | | NQKFKGKATLTVDRSISTAYMHLNRLRSDD | | VPDRFSGSGSGTDFTLTISSLQAEDVAVYYCQ |
| | | TAVYYCTRSHLLRASWFAYWGQGTLVTVSS | | NDYSYPYTFGQGTKLEIKR |
| US10100118B2, | 66 | EVQLVESGGGLVKPGGSLRLSCAASGFTFSS | 67 | QLTQPPSVSAAPGQKVTISCSGSNSNIGNNYV |
| US20190002576A1, | | YSMNWVRQAPGKGLEWVSSISSSSSYIYYAD | | SWYQQLPGTAPKLLIYDNNRRPSGIPDRFSGS |
| US9969807B2 | | SVKGRFTISRDNSKNTLYLQMNSLRAEDTAV | | KSGTSATLGITGLQTGDEADYFCGTWDSSLS |
| | | YYCARAEWFSEALDYWGQGTLVTVSS | | AGVFGGGTKLTVL |
| US10100118B2, | 68 | QVQLVQSGGGLVKPGGSLRLSCAASGFTFSS | 69 | QSVVTQPPSVSAAPGQKVTISCSGSGSNIGNN |
| US20190002576A1, | | YSMNWVRQAPGKGLEWVSSISSSSSYIYYAD | | YVSWYQQLPGTAPKLLIYDNNKRPSGIPDRFS |
| US9969807B2 | | SVKGRFTISRDNAKNSLYLQMNSLRPEDTAV | | GSKSGTSATLGITGLQTGDEADYYCATWDSS |
| | | YYCARESGSDALDIWGRGTMVTVSS | | LSAPWVFGGGTKVTVL |
| US10100118B2, | 70 | EVQLVESGGGLVQPGGSLRLSCAASGFTFSS | 71 | NFMLTQPASVSGSPGQSITISCTGTSADVGGD |
| US20190002576A1, | | YEMNWVRQAPGKGLEWVSSISSSSSYIYYA | | YYVSWYQQHPGKAPKLTIYDVSERPSGVSNR |
| US9969807B2 | | DSVKGRFTISRDNAKNSLYLQMNSLRAEDT | | FSGSKSGNTASLTISGLQTEDEADYYCGSYTS |
| | | AVYYCARADYYEAFDIWGQGTMVTVSS | | SGTWLFGGGTKLTVL |
| US10100118B2, | 72 | EVQLVESGGGLVKPGGSLRLSCAASGFTFSS | 73 | QSALTQPPSVSVAPGQTARITCGGNNIGSKSV |
| US20190002576A1, | | YSMNWVRQAPGKGLEWVSSISSSSSYIYYAD | | HWYQQKPGQAPVLVVYDDSDRPSGIPERFSG |
| US9969807B2 | | SVKGRFTISRDNAKNSLYLQMNSLRAEDTA | | SNSGNTATLTISRVEAGDEADYYCQVWDSSS |
| | | VYYCARAGTRGDAFDIWGQGTMVTVSS | | DHLVFGGGTKVTVL |
| US10100118B2, | 74 | QVQLVQSGAEVKKPGASVKVSCKASGYTFTSYG | 75 | QLVLTQPPSVSAAPGQKVTISCSGSSSNIGNN |
| US20190002576A1, | | ISWVRQAPGQGLEWMGWISAYNGNTKYSQKLR | | YVSWYQQLPGTAPKLLIYDNNKRPSGIPDRFS |
| US9969807B2 | | GRVTMTRDTSTSTAYMELRSLRSDDTAVYYCAR | | GSKSGTSATLGITGLQTGDEADYYCGTWDSS |
| | | DEEYDFWSGYGSWYYYYGMDVWGQGTTVTVSS | | LSAVFGGGTKLTVL |
| US10100118B2, | 76 | EVQLVDSGGGLVKPGGSLRLSCAASGFTFSS | 77 | QAGLTQPPSVSAAPGQQFTISCSGSSSNIGKNY |
| US20190002576A1, | | YSMNWVRQAPGKGLEWVSSISSSSSYIYYAD | | VSWYQQLPGSAPKLLIYDNHKRPSGIPDRFSG |
| US9969807B2 | | SVKGRFTISRDNAKNSLYLQMNSLRAEDTA | | SKSGTSATLGITGLQTGDEADYYCGTWDDSL |
| | | VYYCAREDYYDSIDYWGQGTLVTVSS | | SGWVFGGGTKLTVL |
| US10100118B2, | 78 | EVQLVESGGGLVQPGGSLRLSCAASGFTFSS | 79 | QAGLTQPPSASGTPGQRVTIACSGSSSNIGTYT |
| US20190002576A1, | | YEMNWVRQAPGKGLEWVSYISSSGSTIYYA | | VNWYQHVPGTAPKLLIYSTYQRPLEVPDRFS |
| US9969807B2 | | DSVKGRFTISRDNAKNSLYLQMNSLRAEDT | | GSKSGTSASLAISGLRSEDEGDYYCASWDDR |
| | | AVYYCARVQQWPDDAFDIWGQGTMVTVSS | | LNGFYVFGSGTKVTVL |
| US10100118B2, | 80 | EVQLVQSGGGVVQPGRSLRVSCAASGFTFSS | 81 | QSVLTQPRSVSGSPGQSVTISCTGTSIDVDKD |
| US20190002576A1, | | YGMHWVRQTPGKGLEWVAGIWYDENDKY | | NLVSWYQQHPGRVPKLIIYDVNKRPSGVPDH |
| US9969807B2 | | YADSVKGRFTISRDNSKNTLHLQMNSLRAE | | FSGSKSGTSASLAISGLRSEDEADYYCAAWD |
| | | DTAVYYCARQFRDYYFDVWGRGTLVTVSS | | DSLSSWVFGGGTKVTVL |
| US10100118B2, | 82 | EVQLVQSGGGLVKPGGSLRLSCAASGFTFSN | 83 | LPVLTQPASVSGSPGQSITISCTGTSSDVGRYD |
| US20190002576A1, | | AWMSWVRQAPGKGLEWVGRIKSKTDGGTT | | YVSWYQQHPGKAPQLMIYDVSNRPSGVSNRF |
| US9969807B2 | | DYAAPVKGRFTISRDDSKNTLYLQMNSLKTE | | SGSKSGNTASLTISGLQAEDEADYYCSSYTGS |
| | | DTAVYYCTTDYDFWSGYYYWGQGTTVTVSS | | STLYVFGTGTKVTVL |
| US10100118B2, | 84 | EVQLVQSGAEVKKPGSSVKVSCKASGGTFST | 85 | EVQLVQSGAEVKKPGSSVKVSCKASGGTFST |
| US20190002576A1, | | YAISWVRQAPGQGLEWMGGTIPKFGTANYA | | YAISWVRQAPGQGLEWMGGTIPKFGTANYA |
| US9969807B2 | | QKFQGRVTITADESTSTAYMELSSLRSEDTA | | QKFQGRVTITADESTSTAYMELSSLRSEDTAV |
| | | VYYCARAVVPAAIVEAMDVWGQGTTVTVSS | | YYCARAVVPAAIVEAMDVWGQGTTVTVSS |
| US10100118B2, | 86 | QVQLVQSGAEVKKPGASVKVSCKASGYTLSMY | 87 | QAVLTQPPSVSVAPGKTARITCGGNNIGSKSV |
| US20190002576A1, | | GISWVRHAPGQGLEWMGWINPYTGDRKYAQRF | | HWYQQKPGQAPVLVVYDDSDRPSGIPERFSG |
| US9969807B2 | | QGRLTVTTDTSTATSYMELTSLRSDDTAVYYC | | SNSGNTATLTISRVEAGDEADYYCQVWDSSS |
| | | AREEYHDSMIGYYVGGFDLWGQGTLVTVSS | | DHVVFGGGPQLTVL |
| US10100118B2, | 88 | EVQLLESGGGLVKPGGSLRLSCAASGFTFSS | 89 | QSVLTQPPSVSAAPGHEVTISCSGSSSNIGNNY |
| US20190002576A1, | | YSMNWVRQAPGKGLEWVSSISSSSSYIYYAD | | VSWYQQVPGTAPKLLIYDNNKRASEIPDRFFG |
| US9969807B2 | | SVKGRFTISRDNAKNSLYLQMNSLRAEDTA | | SKSGTSATLGVSGLQTGDEADYYCGTWDSSL |
| | | VYYCARANWDAFDIWGQGTMVTVSS | | NDVVFGGGTKLTVL |
| US10100118B2, | 90 | EVQLVESGGGLVKPGGSLRLSCAASGFTFSN | 91 | LPVLTQSASVSGSPGQSITISCTGTSSDVGRYD |
| US20190002576A1, | | AWMSWVRQAPGKGLEWVGRIKSKTDGGTT | | YVSWYQQHPGKAPQLMIYDVSNRPSGVSNRF |
| US9969807B2 | | DYAAPVKGRFTISRDDSKNTLYLQMNSLKTE | | SGSKSGNTASLTISGLQAEDEADYYCSSYTGS |
| | | DTAVYYCTTDYDFWSGYYYWGQGTLVTVSS | | STLYVFGTGTKVTVL |
| US10100118B2, | 92 | EVQLVQSGAEVKKPGASVKVSCKASGYTFTGY | 93 | DIQLTQSPSSLSASVGDRVTITCRASQSISDYL |
| US20190002576A1, | | YMHWVRQAPGQGLEWMGWISAYNGNTNYAQK | | NWYHQKPGKAPRLLIYAASSLQSGVPSRFSG |
| US9969807B2 | | LQGRVTMTTDTSTSTAYMELRSLRSDDTAVYY | | TRSGTDFTLTINNLQPEDSATYYCQQSYSTPL |
| | | CAREEDYYGSGEHYYFDYWGQGTLVTVSS | | TFGGGTKVDIK |
| US9815901 | 94 | QVQLVQSGAEVKKPGASVKVSCKASGYTFT | 95 | DIQMTQSPSSLSASVGDRVTITCRASQSISTYL |
| | | GYYMHWVRQAPGQGLEWMGWINPNSGGTNY | | NWYQQKPGKAPNLLIYAAFSLQSGVPSRFSG |
| | | AQKFQGRVTMTRDTSISTAYMELSRLRSDDT | | SGSGTDFTLTINSLQPEDFATYYCQQGDSVPL |
| | | AVYYCARDMNILATVPFDIWGQGTMVTVSS | | TFGGGTKLEIK |
| US9815901 | 96 | QVQLVQSGAEVKKPGASVKVSCKASGYTFT | 97 | DIQMTQSPSSLSASVGDRVTITCRASQSISSYL |
| | | GYYMHWVRQAPGQGLEWMGWINPNSGGTNY | | NWYQQKPGKAPKLLIYAASSLQSGVPSRFSG |
| | | AQKFQGRVTLTRDTSISTVYMELSRLRSDDT | | SGSGTDFTLTVNSLQPEDFATYYCQQGDSVPL |
| | | AVYYCARDMNILATVPFDIWGQGTMVTVSS | | TFGGGTRLEIK |
| US9815901 | 98 | QVQLVQSGAEVKKPGASVKVSCKASGYIFT | 99 | DIQLTQSPSSLSASVGDRVTITCRASQSISSYL |
| | | GYYIHWVRQAPGQGLEWMGWINPNSGGTNY | | NWYQQKPGKAPKLLIYAASSLQSGVPSRFSG |
| | | AQKFQGRVTMTRDTSISTAYMELSGLRSDDP | | SGSGTDFTLTVNSLQPEDFATYYCQQGDSVPL |
| | | AVYYCARDMNILATVPFDIWGQGTLVTVSS | | TFGGGTKVEIK |
| US9815901 | 100 | QVQLQQSGAEVKKSGASVKVSCKASGYTFT | 101 | DIQMTQSPSSLSASVGDRVTITCRASQSISSYL |
| | | DYYMHWLRQAPGQGLEWMGWINPNSGDTNY | | NWYQQKPGKAPKLLIYAASSLQSGVPSRFSG |
| | | AQKFQGRVTLTRDTSISTVYMELSRLRSDDT | | SGSGTDFTLTISSLQPEDFATYYCQQGDSVPL |
| | | AVYYCARDMNILATVPFDIWGQGTMVTVSS | | TFGGGTKVEIK |
| US8569461B2 | 102 | EVQLVQSGAEVKKPGESLKISCKGSGYSFTD | 103 | DIVMTQSPDSLAVSLGERATINCESSQSLLNS |
| | | YYMKWARQMPGKGLEWMGDIIPSNGATFY | | GNQKNYLTWYQQKPGQPPKPLIYWASTRESG |
| | | NQKFKGQVTISADKSISTTYLQWSSLKASDT | | VPDRFSGSGSGTDFTLTISSLQAEDVAVYYCQ |
| | | AMYYCARSHLLRASWFAYWGQGTMVTVSS | | NDYSYPYTFGQGTKLEIKR |
| US9822181B2 | 107 | QVQLVQSGAELKKPGASVKVSCKASGYTFT | 108 | DFVMTQSPDSLAVSLGERVTMSCKSSQSLLN |
| | | DYYMKWVRQAPGQGLEWIGDIIPSNGATFY | | SGNQKNYLTWYQQKPGQPPKLLIYWASTRES |
| | | NQKFKGRVTITVDKSTSTAYMELSSLRSEDT | | GVPDRFSGSGSGTDFTLTISSLQAEDVAVYYC |
| | | AVYYCARSHLLRASWFAYWGQGTLVTVSS | | QNDYSYPYTFGQGTKLEIK |
| US9822181 | 109 | EVQLVQSGAELKKPGASVKVSCKASGYTFT | 110 | DFVMTQSPDSLAVSLGERVTMSCKSSQSLLN |
| | | DYYMKWVRQAPGQGLEWIGDIIPSNGATFY | | SGNQKNYLTWYQQKPGQPPKLLIYWASTRES |
| | | NQKFKGRVTITVDKSTSTAYMELSSLRSEDT | | GVPDRFSGSGSGTDFTLTISSLQAEDVAVYYC |
| | | AVYYCARSHLLRASWFAYWGQGTLVTVSS | | QNDYSYPYTFGQGTKLEIK |
| US20170152321A1 | 111 | QVQLQQPGAELVRPGASVKLSCKASGYTFTS | 112 | DVQITQSPSYLAASPGETITINCRASKSISKDL |
| | | YWMNWVKQRPDQGLEWIGRIDPYDSETHY | | AWYQEKPGKTNKLLIYSGSTLQSGIPSRFSGS |
| | | NQKFKDKAILTVDKSSSTAYMQLSSLTSEDS | | GSGTDFTLTISSLEPEDFAMYYCQQHNKYPYT |
| | | AVYYCARGNWDDYWGQGTTLTVSS | | FGGGTKLEIK |
| US20170152321A1 | 113 | QIQLVQSGPELKKPGETVKISCKASGYIFTNY | 114 | DIVLTQSPASLAVSLGQRATISCRASESVDNY |
| | | GMNWVKQAPGKSFKWMGWINTYTGESTYS | | GNTFMHWYQQKPGQPPKLLIYRASNLESGIP |
| | | ADFKGRFAFSLETSASTAYLHINDLKNEDTA | | ARFSGSGSRTDFTLTINPVEADDVATYYCQQS |
| | | TYFCARSGGYDPMDYWGQGTSVTVSS | | NEDPPTFGAGTKLELK |
| Mouse 7G3 as | 115 | EVQLQQSGPELVKPGASVKMSCKASGYTFT | 116 | DFVMTQSPSSLTVTAGEKVTMSCKSSQSLLNS |
| reported in | | DYYMKWVKQSHGKSLEWIGDIIPSNGATFY | | GNQKNYLTWYLQKPGQPPKLLIYWASTRESG |
| US20160046718 | | NQKFKGKATLTVDRSSSTAYMHLNSLTSEDS | | VPDRFTGSGSGTDFTLTISSVQAEDLAVYYCQ |
| | | AVYYCTRSHLLRASWFAYWGQGTLVTVSA | | NDYSYPYTFGGGTKLEIKR |
| WO2019060707A1 | 117 | QVQLVQSGAEVKKPGASVKVSCKASGYIFTS | 118 | DIQMTQSPSSLSASVGDRVTITCRASQDINSYL |
| | | SIMHWVRQAPGQGLEWIGYIKPYNDGTKYN | | SWFQQKPGKAPKTLIYRVNRLVDGVPSRFSG |
| | | EKFKGRATLTSDRSTSTAYMELSSLRSEDTA | | SGSGNDYTLTISSLQPEDFATYYCLQYDAFPY |
| | | VYYCAREGGNDYYDTMDYWGQGTLVTVSS | | TFGQGTKVEIKR |

TABLE 9
Additional anti-CD3 VH and VL Sequences

| | SEQ | | SEQ | |
|---|---|---|---|---|
| Citation | ID | VH | ID | VL |
| WO2018208864 | 119 | EVQLLESGGGLVQPGGSLRLSCAASGFTFDT | 120 | QTVVTQEPSLSVSPGGTVTLTCGSSTGAVTTS |
| | | YAMNWVRQAPGKGLEWVARIRSKYNNYAT | | NYANWVQQTPGQAPRGLIGGTDKRAPGVPD |
| | | YYADSVKDRFTISRDDSKSTLYLQMESLRAE | | RFSGSLLGDKAALTITGAQAEDEADYYCALW |
| | | DTAVYYCVRHANFGAGYVSWFAHWGQGTL | | YSNHWVFGGGTKLTVL |
| | | VTVSS | | |
| WO2018208864 | 121 | EVQLLESGGGLVQPGGSLRLSCAASGFTFDT | 122 | QTVVTQEPSLSVSPGGTVTLTCGSSTGAVTTS |
| | | YAMNWVRQAPGKGLEWVARIRSKYNNYAT | | NYANWVQQTPGQAPRGLIGGTDKRAPGVPD |
| | | YYADSVKDRFTISRDDSKSTLYLQMESLRAE | | RFSGSLLGDKAALTITGAQAEDEADYYCALW |
| | | DTAVYYCVRHANFGAGYVSWFAHWGQGTL | | YSDLWVFGGGTKLTVL |
| | | VTVSS | | |
| WO2018208864 | 123 | QVQLVQSGAEVKKPGASVKVSCKASGFNIK | 124 | DIVMTQSPDSLAVSLGERATINCKSSQSLLNA |
| | | DYYMHWVRQAPGQRLEWMGWIDLENANTI | | RTGKNYLAWYQQKPGQPPKLLIYWASTRESG |
| | | YDAKFQGRVTITRDTSASTAYMELSSLRSED | | VPDRFSGSGSGTDFTLTISSLQAEDVAVYYCK |
| | | TAVYYCARDAYGRYFYDVWGQGTLVTVSS | | QSYSRRTFGGGTKVEIK |
| WO2018208864 | 125 | QVQLVQSGAEVKKPGASVKVSCKASGFNIK | 126 | DIVMTQSPDSLAVSLGERATINCKSSQSLLNA |
| | | DYYMHWVRQAPGQRLEWIGWIDLENANTV | | RTGKNYLAWYQQKPGQPPKLLIYWASTRESG |
| | | YDAKFQGRVTITRDTSASTAYMELSSLRSED | | VPDRFSGSGSGTDFTLTISSLQAEDVAVYYCK |
| | | TAVYYCARDAYGRYFYDVWGQGTLVTVSS | | QSYFRRTFGGGTKVEIK |
| WO2018208864 | 127 | QVQLVQSGAEVKKPGASVKVSCKASGFNIK | 128 | DIVMTQSPDSLAVSLGERATINCKSSQSLLNA |
| | | DYYMHWVRQAPGQRLEWIGWIDLENANTV | | RTGKNYLAWYQQKPGQPPKLLIYWASTRESG |
| | | YDAKFQGRVTITRDTSASTAYMELSSLRSED | | VPDRFSGSGSGTDFTLTISSLQAEDVAVYYCT |
| | | TAVYYCARDAYGQYFYDVWGQGTLVTVSS | | QSYFRRTFGGGTKVEIK |

1. A multimeric, bispecific or multispecific binding molecule comprising two or five bivalent binding units and a modified J-chain,
wherein the modified J-chain comprises a wild-type J-chain or a functional fragment or variant thereof and a J-chain-associated antigen-binding domain that specifically binds to an immune effector cell,
wherein each binding unit comprises two antibody heavy chains, each comprising an IgA, IgA-like, IgM, or IgM-like heavy chain constant region or multimerizing fragment thereof and at least a heavy chain variable region (VH) portion of a binding unit-associated antigen-binding domain, wherein at least three of the binding unit-associated antigen-binding domains specifically bind to CD123, and
wherein the binding molecule can induce immune effector cell-dependent killing of cells expressing CD123.

2. The binding molecule of claim 1, wherein the modified J-chain comprises a variant J-chain or fragment thereof comprising one or more single amino acid substitutions, deletions, or insertions relative to a wild-type J-chain that can affect serum half-life of the binding molecule; and wherein the binding molecule exhibits an increased serum half-life upon administration to an animal relative to a reference binding molecule that is identical except for the one or more single amino acid substitutions, deletions, or insertions in the J-chain, and is administered in the same way to the same animal species.

3. The binding molecule of claim 2, wherein the modified J-chain comprises an amino acid substitution at the amino acid position corresponding to amino acid Y102 of the mature wild-type human J-chain (SEQ ID NO: 2).

4. The binding molecule of claim 3, wherein the amino acid corresponding to Y102 of SEQ ID NO: 2 is substituted with alanine (A), serine (S), or arginine (R).

5. The binding molecule of claim 4, wherein the amino acid corresponding to Y102 of SEQ ID NO: 2 is substituted with alanine (A).

6. The binding molecule of claim 5, wherein the J-chain is a variant human J-chain and comprises the amino acid sequence SEQ ID NO: 3 (“J*”).

7. The binding molecule of claim 1, wherein the J-chain-associated antigen-binding domain comprises an antibody single chain Fv (scFv) fragment fused or chemically conjugated to the J-chain or fragment or variant thereof.

8. The binding molecule of claim 7, wherein the scFv fragment is fused to the J-chain via a peptide linker.

9. The binding molecule of claim 8, wherein the scFv fragment is fused to the N-terminus of the J-chain or fragment or variant thereof, the C-terminus of the J-chain or fragment or variant thereof, or to both the N-terminus and C-terminus of the J-chain or fragment or variant thereof.

10. (canceled)

11. (canceled)

12. The binding molecule of claim 71, wherein the immune effector cell is a CD8+ cytotoxic T cell, and wherein the scFv fragment specifically binds to CD3.

13. The binding molecule of claim 12, wherein the scFv fragment comprises a heavy chain variable region (VH) and a light chain variable region (VL), wherein the VH comprises VH complementarity-determining regions VHCDR1, VHCDR2, and VHCDR3 and the VL comprises VL complementarity-determining regions VLCDR1, VLCDR2, and VLCDR3, wherein
(a) the VHCDR1, VHCDR2, and VHCDR3 comprise the amino acid sequences SEQ ID NO: 5, SEQ ID NO: 6, and SEQ ID NO: 7 with zero, one, or two amino acid substitutions, respectively, and the VLCDR1, VLCDR2, and VLCDR3 comprise the amino acid sequences SEQ ID NO: 9, SEQ ID NO: 10, and SEQ ID NO: 11 with zero, one, or two amino acid substitutions, respectively;
(b) the VHCDR1, VHCDR2, and VHCDR3 comprise the amino acid sequences SEQ ID NO: 130, SEQ ID NO: 132, and SEQ ID NO: 135 with zero, one, or two amino acid substitutions, respectively, and the VLCDR1, VLCDR2, and VLCDR3 comprise the amino acid sequences SEQ ID NO: 138, SEQ ID NO: 140, and SEQ ID NO: 142 with zero, one, or two amino acid substitutions, respectively;
(c) the VHCDR1, VHCDR2, and VHCDR3 comprise the amino acid sequences SEQ ID NO: 130, SEQ ID NO: 132, and SEQ ID NO: 135 with zero, one, or two amino acid substitutions, respectively, and the VLCDR1, VLCDR2, and VLCDR3 comprise the amino acid sequences SEQ ID NO: 138, SEQ ID NO: 140, and SEQ ID NO: 143 with zero, one, or two amino acid substitutions, respectively;
(d) the VHCDR1, VHCDR2, and VHCDR3 comprise the amino acid sequences SEQ ID NO: 131, SEQ ID NO: 133, and SEQ ID NO: 136 with zero, one, or two amino acid substitutions, respectively, and the VLCDR1, VLCDR2, and VLCDR3 comprise the amino acid sequences SEQ ID NO: 139, SEQ ID NO: 141, and SEQ ID NO: 144 with zero, one, or two amino acid substitutions, respectively;
(e) the VHCDR1, VHCDR2, and VHCDR3 comprise the amino acid sequences SEQ ID NO: 131, SEQ ID NO: 134, and SEQ ID NO: 136 with zero, one, or two amino acid substitutions, respectively, and the VLCDR1, VLCDR2, and VLCDR3 comprise the amino acid sequences SEQ ID NO: 139, SEQ ID NO: 141, and SEQ ID NO: 145 with zero, one, or two amino acid substitutions, respectively; or
(f) the VHCDR1, VHCDR2, and VHCDR3 comprise the amino acid sequences SEQ ID NO: 131, SEQ ID NO: 134, and SEQ ID NO: 137 with zero, one, or two amino acid substitutions, respectively, and the VLCDR1, VLCDR2, and VLCDR3 comprise the amino acid sequences SEQ ID NO: 139, SEQ ID NO: 141, and SEQ ID NO: 146 with zero, one, or two amino acid substitutions, respectively.

14. The binding molecule of claim 13, wherein the scFv fragment comprises the VH and VL amino acid sequences SEQ ID NO: 4 and SEQ ID NO: 8, SEQ ID NO: 119 and SEQ ID NO: 120, SEQ ID NO: 121 and SEQ ID NO: 122, SEQ ID NO: 123 and SEQ ID NO: 124, SEQ ID NO: 125 and SEQ ID NO: 126, or SEQ ID NO: 127 and SEQ ID NO: 128, respectively.

15. The binding molecule of claim 12, wherein the scFv fragment comprises a heavy chain variable region (VH) and a light chain variable region (VL), wherein the VH and VL comprise the amino acid sequences SEQ ID NO: 13 and SEQ ID NO: 14, respectively.

16. The binding molecule of claim 13 wherein the modified J chain comprises an amino acid sequence comprising amino acids 20 to 420 of SEQ ID NO: 12, amino acids 20 to 412 of SEQ ID NO: 15, or amino acids 23 to 415 of SEQ ID NO: 16.

17. The binding molecule of claim 1, wherein the immune effector cell is an NK cell, and wherein the scFv fragment specifically binds to CD16.

18-24. (canceled)

25. The binding molecule of claim 1, wherein all the binding unit-associated antigen binding domains are identical.

26. The binding molecule of claim 25, wherein the binding unit-associated antigen-binding domains comprise a heavy chain variable region (VH) and a light chain variable region (VL), wherein the VH and VL comprise six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3 comprise the CDRs of an antibody comprising the VH and VL amino acid sequences comprising or contained within SEQ ID NO: 32 and SEQ ID NO: 33, SEQ ID NO: 37 and SEQ ID NO: 38, SEQ ID NO: 42 and SEQ ID NO: 43, SEQ ID NO: 44 and SEQ ID NO: 45, SEQ ID NO: 46 and SEQ ID NO: 47, SEQ ID NO: 48 and SEQ ID NO: 49, SEQ ID NO: 50 and SEQ ID NO: 51, SEQ ID NO: 52 and SEQ ID NO: 53, SEQ ID NO: 54 and SEQ ID NO: 55, SEQ ID NO: 56 and SEQ ID NO: 57, SEQ ID NO: 58 and SEQ ID NO: 59, SEQ ID NO: 60 and SEQ ID NO: 61, SEQ ID NO: 62 and SEQ ID NO: 63, SEQ ID NO: 64 and SEQ ID NO: 65, SEQ ID NO: 66 and SEQ ID NO: 67, SEQ ID NO: 68 and SEQ ID NO: 69, SEQ ID NO: 70 and SEQ ID NO: 71, SEQ ID NO: 72 and SEQ ID NO: 73, SEQ ID NO: 74 and SEQ ID NO: 75, SEQ ID NO: 76 and SEQ ID NO: 77, SEQ ID NO: 78 and SEQ ID NO: 79, SEQ ID NO: 80 and SEQ ID NO: 81, SEQ ID NO: 82 and SEQ ID NO: 83, SEQ ID NO: 84 and SEQ ID NO: 85, SEQ ID NO: 86 and SEQ ID NO: 87, SEQ ID NO: 88 and SEQ ID NO: 89, SEQ ID NO: 90 and SEQ ID NO: 91, SEQ ID NO: 92 and SEQ ID NO: 93, SEQ ID NO: 94 and SEQ ID NO: 95, SEQ ID NO: 96 and SEQ ID NO: 97, SEQ ID NO: 98 and SEQ ID NO: 99, SEQ ID NO: 100 and SEQ ID NO: 101, SEQ ID NO: 102 and SEQ ID NO: 103, SEQ ID NO: 107 and SEQ ID NO: 108, SEQ ID NO: 109 and SEQ ID NO: 110, SEQ ID NO: 111 and SEQ ID NO: 112, SEQ ID NO: 113 and SEQ ID NO: 114, SEQ ID NO: 115 and SEQ ID NO: 116, or SEQ ID NO: 117 and SEQ ID NO: 118, respectively or the CDRs of an antibody comprising the VH and VL amino acid sequences comprising or contained within SEQ ID NO: 32 and SEQ ID NO: 33, SEQ ID NO: 37 and SEQ ID NO: 38, SEQ ID NO: 42 and SEQ ID NO: 43, SEQ ID NO: 44 and SEQ ID NO: 45, SEQ ID NO: 46 and SEQ ID NO: 47, SEQ ID NO: 48 and SEQ ID NO: 49, SEQ ID NO: 50 and SEQ ID NO: 51, SEQ ID NO: 52 and SEQ ID NO: 53, SEQ ID NO: 54 and SEQ ID NO: 55, SEQ ID NO: 56 and SEQ ID NO: 57, SEQ ID NO: 58 and SEQ ID NO: 59, SEQ ID NO: 60 and SEQ ID NO: 61, SEQ ID NO: 62 and SEQ ID NO: 63, SEQ ID NO: 64 and SEQ ID NO: 65, SEQ ID NO: 66 and SEQ ID NO: 67, SEQ ID NO: 68 and SEQ ID NO: 69, SEQ ID NO: 70 and SEQ ID NO: 71, SEQ ID NO: 72 and SEQ ID NO: 73, SEQ ID NO: 74 and SEQ ID NO: 75, SEQ ID NO: 76 and SEQ ID NO: 77, SEQ ID NO: 78 and SEQ ID NO: 79, SEQ ID NO: 80 and SEQ ID NO: 81, SEQ ID NO: 82 and SEQ ID NO: 83, SEQ ID NO: 84 and SEQ ID NO: 85, SEQ ID NO: 86 and SEQ ID NO: 87, SEQ ID NO: 88 and SEQ ID NO: 89, SEQ ID NO: 90 and SEQ ID NO: 91, SEQ ID NO: 92 and SEQ ID NO: 93, SEQ ID NO: 94 and SEQ ID NO: 95, SEQ ID NO: 96 and SEQ ID NO: 97, SEQ ID NO: 98 and SEQ ID NO: 99, SEQ ID NO: 100 and SEQ ID NO: 101, SEQ ID NO: 102 and SEQ ID NO: 103, SEQ ID NO: 107 and SEQ ID NO: 108, SEQ ID NO: 109 and SEQ ID NO: 110, SEQ ID NO: 111 and SEQ ID NO: 112, SEQ ID NO: 113 and SEQ ID NO: 114, SEQ ID NO: 115 and SEQ ID NO: 116, or SEQ ID NO: 117 and SEQ ID NO: 118, respectively, except for one or two amino acid substitutions in one or more of the CDRs.

27. (canceled)

28. The binding molecule of claim 25, which is a dimeric binding molecule comprising two bivalent binding units, wherein each binding unit comprises two antibody heavy chains, each comprising an IgA or IgA-like heavy chain constant region or multimerizing fragment thereof.

29-34. (canceled)

35. The binding molecule of claim 26, which is a pentameric binding molecule comprising five bivalent binding units, wherein each binding unit comprises two IgM or IgM-like heavy chain constant regions or multimerizing fragments thereof.

36. (canceled)

37. (canceled)

38. The binding molecule of claim 35, wherein the IgM or IgM-like heavy chain constant regions are human IgM constant regions, and wherein each IgM heavy chain constant region is a human IgM constant region or multimerizing variant or fragment thereof, comprising the amino acid sequence SEO ID NO: 22, SEO ID NO: 23, or a multimerizing variant or fragment thereof.

39. (canceled)

40. (canceled)

41. The binding molecule of claim 38, wherein each IgM heavy chain constant region comprises a variant of the amino acid sequence SEQ ID NO: 22 or SEQ ID NO: 23, wherein the variant comprises an amino acid substitution at position P311 of SEQ ID NO: 22 or SEQ ID NO: 23, an amino acid substitution at position P313 of SEQ ID NO: 22 or SEQ ID NO: 23, or amino acid substitutions at positions P311 and P313 of SEQ ID NO: 22 or SEQ ID NO: 23.

42-45. (canceled)

46. A polynucleotide comprising a nucleic acid sequence that encodes a polypeptide subunit of the binding molecule of claim 1.

47-58. (canceled)

59. A host cell comprising the polynucleotide of claim 46, wherein the host cell can express the binding molecule, or a subunit thereof.

60. A method of producing a binding molecule, comprising culturing the host cell of claim 59, and recovering the binding molecule.

61. A method of treating cancer or other malignancy, comprising administering to a subject in need of treatment an effective amount of the binding molecule of claim 1, wherein the binding molecule can induce immune effector cell-mediated killing of cancer cells.

62-66. (canceled)


SEQUENCE LISTING (146 sequences)

SEQ ID NO: 1 - protein, 159 aa, Homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 2 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 3 - protein, 137 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 4 - protein, 125 aa, Artificial Sequence
  Description of Artificial Sequence Synthetic polypeptide
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSS

SEQ ID NO: 5 - protein, 5 aa, Artificial Sequence
  Description of Artificial Sequence Synthetic peptide
TYAMN

SEQ ID NO: 6 - protein, 19 aa, Artificial Sequence
  Description of Artificial Sequence Synthetic peptide
RIRSKYNNYATYYADSVKD

SEQ ID NO: 7 - protein, 14 aa, Artificial Sequence
  Description of Artificial Sequence Synthetic peptide
HGNFGNSYVSWFAY

SEQ ID NO: 8 - protein, 109 aa, Artificial Sequence
  Description of Artificial Sequence Synthetic polypeptide
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 9 - protein, 14 aa, Artificial Sequence
  Description of Artificial Sequence Synthetic peptide
RSSTGAVTTSNYAN

SEQ ID NO: 10 - protein, 7 aa, Artificial Sequence
  Description of Artificial Sequence Synthetic peptide
GTNKRAP

SEQ ID NO: 11 - protein, 9 aa, Artificial Sequence
  Description of Artificial Sequence Synthetic peptide
ALWYSNLWV

SEQ ID NO: 12 - protein, 420 aa, Artificial Sequence
  Description of Artificial Sequence Synthetic polypeptide
MGWSYIILFLVATATGVHSEVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 13 - protein, 120 aa, Artificial Sequence [sequence omitted]

SEQ ID NO: 14 - protein, 106 aa, Artificial Sequence
  Description of Artificial Sequence Synthetic polypeptide
DIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKLEIK

SEQ ID NO: 15 - protein, 412 aa, Artificial Sequence
  Description of Artificial Sequence Synthetic polypeptide
MGWSYIILFLVATATGVHSQVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKLEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 16 - protein, 415 aa, Artificial Sequence
  Description of Artificial Sequence Synthetic polypeptide
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSQVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKLEIK

SEQ ID NO: 17 - protein, 5 aa, Artificial Sequence
  Description of Artificial Sequence Synthetic peptide
GGGGS

SEQ ID NO: 18 - protein, 10 aa, Artificial Sequence
  Description of Artificial Sequence Synthetic peptide
GGGGSGGGGS

SEQ ID NO: 19 - protein, 15 aa, Artificial Sequence
  Description of Artificial Sequence Synthetic peptide
GGGGSGGGGSGGGGS

SEQ ID NO: 20 - protein, 20 aa, Artificial Sequence
  Description of Artificial Sequence Synthetic peptide
GGGGSGGGGSGGGGSGGGGS

SEQ ID NO: 21 - protein, 25 aa, Artificial Sequence
  Description of Artificial Sequence Synthetic peptide
GGGGSGGGGSGGGGSGGGGSGGGGS

SEQ ID NO: 22 - protein, 453 aa, Homo sapiens [sequence omitted]

SEQ ID NO: 23 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 24 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 25 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 26 - protein, 764 aa, Homo sapiens
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 27 - protein, 585 aa, Homo sapiens
KSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPR

SEQ ID NO: 28 - protein, 378 aa, Homo sapiens
MVLLWLTLLLIALPCLLQTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 29 - protein, 300 aa, Homo sapiens
MVLLWLTLLLIALPCLLQTKEGGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 30 - protein, 378 aa, Macaca fascicularis
MTLLWLTLLLVATPCLLRTKEDPNAPIRNLRMKEKAQQLMWDLNRNVTDVECIKGTDYSMPAMNNSYCQFGAISLCEVTNYTVRVASPPFSTWILFPENSGTPRAGAENLTCWVHDVDFLSCSWVVGPAAPADVQYDLYLNNPNSHEQYRCLHYKTDARGTQIGCRFDDIAPLSRGSQSSHILVRGRSAAVSIPCTDKFVFFSQIERLTPPNMTGECNETHSFMHWKMKSHFNRKFRYELRIQKRMQPVRTEQVRDTTSFQLPNPGTYTVQIRARETVYEFLSAWSTPQRFECDQEEGASSRAWRTSLLIALGTLLALLCVFLICRRYLVMQRLFPRIPHMKDPIGDTFQQDKLVVWEAGKAGLEECLVSEVQVVEKT

SEQ ID NO: 31 - protein, 396 aa, Mus musculus
MAANLWLILGLLASHSSDLAAVREAPPTAVTTPIQNLHIDPAHYTLSWDPAPGADITTGAFCRKGRDIFVWADPGLARCSFQSLSLCHVTNFTVFLGKDRAVAGSIQFPPDDDGDHEAAAQDLRCWVHEGQLSCQWERGPKATGDVHYRMFWRDVRLGPAHNRECPHYHSLDVNTAGPAPHGGHEGCTLDLDTVLGSTPNSPDLVPQVTITVNGSGRAGPVPCMDNTVDLQRAEVLAPPTLTVECNGSEAHARWVARNRFHHGLLGYTLQVNQSSRSEPQEYNVSIPHFWVPNAGAISFRVKSRSEVYPRKLSSWSEAWGLVCPPEVMPVKTALVTSVATVLGAGLVAAGLLLWWRKSLLYRLCPPIPRLRLPLAGEMVVWEPALEDCEVTPVTDA

SEQ ID NO: 32 - protein, 120 aa, Artificial Sequence
  Description of Artificial Sequence Synthetic polypeptide
QVQLQQSGAEVKKPGASVKVSCKASGYTFTDYYMKWVKQSHGKSLEWMGDIIPSNGATFYNQKFKGKATLTVDRSTSTAYMELSSLRSEDTAVYYCARSHLLRASWFAYWGQGTLVTVSS

SEQ ID NO: 33 - protein, 113 aa, Artificial Sequence
  Description of Artificial Sequence Synthetic polypeptide
DFVMTQSPDSLAVSLGERATINCKSSQSLLNTGNQKNYLTWYQQKPGQPPKLLIYWASTRESGVPDRFTGSGSGTDFTLTISSLQAEDVAVYYCQNDYSYPYTFGGGTKLEIK

SEQ ID NO: 34 - protein, 469 aa, Artificial Sequence
  Description of Artificial Sequence Synthetic polypeptide
MGWSYIILFLVATATGVHSQVQLQQSGAEVKKPGASVKVSCKASGYTFTDYYMKWVKQSHGKSLEWMGDIIPSNGATFYNQKFKGKATLTVDRSTSTAYMELSSLRSEDTAVYYCARSHLLRASWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 592 aa, Artificial Sequence
  Description of Artificial Sequence Synthetic polypeptide
MGWSYIILFLVATATGVHSQVQLQQSGAEVKKPGASVKVSCKASGYTFTDYYMKWVKQSHGKSLEWMGDIIPSNGATFYNQKFKGKATLTVDRSTSTAYMELSSLRSEDTAVYYCARSHLLRASWFAYWGQGTLVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 36 - protein, 240 aa, Artificial Sequence
  Description of Artificial Sequence Synthetic polypeptide
MRVPAQLLGLLLLWLRGARCDFVMTQSPDSLAVSLGERATINCKSSQSLLNTGNQKNYLTWYQQKPGQPPKLLIYWASTRESGVPDRFTGSGSGTDFTLTISSLQAEDVAVYYCQNDYSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 115 aa, Artificial Sequence
  Description of Artificial Sequence Synthetic polypeptide
QVQLVQSGAEVKKPGASVKVSCKASGYTFTSYWMNWVRQAPGQGLEWMGRIDPYDSETHYNQKFKDRVTMTVDKSTSTAYMELSSLRSEDTAVYYCARGNWDDYWGQGTTVTVSS

SEQ ID NO: 38 - protein, 107 aa, Artificial Sequence
  Description of Artificial Sequence Synthetic polypeptide
EVVLTQSPATLSLSPGERATLSCRASKSISKDLAWYQQKPGQAPRLLIYSGSTLQSGIPARFSGSGSGTDFTLTISSLEPEDFAVYYCQQHNKYPYTFGGGTKVEIK

SEQ ID NO: 39 - protein, 464 aa, Artificial Sequence
  Description of Artificial Sequence Synthetic polypeptide
MGWSYIILFLVATATGVHSQVQLVQSGAEVKKPGASVKVSCKASGYTFTSYWMNWVRQAPGQGLEWMGRIDPYDSETHYNQKFKDRVTMTVDKSTSTAYMELSSLRSEDTAVYYCARGNWDDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 587 aa, Artificial Sequence
  Description of Artificial Sequence Synthetic polypeptide
MGWSYIILFLVATATGVHSQVQLVQSGAEVKKPGASVKVSCKASGYTFTSYWMNWVRQAPGQGLEWMGRIDPYDSETHYNQKFKDRVTMTVDKSTSTAYMELSSLRSEDTAVYYCARGNWDDYWGQGTTVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 41 - protein, 234 aa, Artificial Sequence
  Description of Artificial Sequence Synthetic polypeptide
MRVPAQLLGLLLLWLRGARCEVVLTQSPATLSLSPGERATLSCRASKSISKDLAWYQQKPGQAPRLLIYSGSTLQSGIPARFSGSGSGTDFTLTISSLEPEDFAVYYCQQHNKYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 120 aa, Artificial Sequence [sequence omitted]

SEQ ID NO: 43 - protein, 113 aa, Artificial Sequence [sequence omitted]

SEQ ID NO: 44 - protein, 115 aa, Artificial Sequence
  Description of Artificial Sequence Synthetic polypeptide
QVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSS

SEQ ID NO: 45 - protein, 107 aa, Artificial Sequence
  Description of Artificial Sequence Synthetic polypeptide
DVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIK

SEQ ID NO: 46 - protein, 120 aa, Artificial Sequence
  Description of Artificial Sequence Synthetic polypeptide
QVQLVQSGAEVKKPGASVKMSCKASGYTFTDYYMKWVKQAPGQGLEWIGDIIPSNGATFYNQKFKGKATLTVDRSISTAYMHLNRLRSDDTAVYYCTRSHLLRASWFAYWGQGTLVTVSS

SEQ ID NO: 47 - protein, 114 aa, Artificial Sequence
  Description of Artificial Sequence Synthetic polypeptide
DFVMTQSPDSLAVSLGERATINCKSSQSLLNSGNQKNYLTWYLQKPGQPPKLLIYWASTRESGVPDRFSGSGSGTDFTLTISSLQAEDVAVYYCQNDYSYPYTFGQGTKLEIKR

SEQ ID NO: 48 - protein, 120 aa, Artificial Sequence
  Description of Artificial Sequence Synthetic polypeptide
QVQLVQSGAEVKKPGSSVKVSCKASGGTFSDYYMKWVRQAPGQGLEWMGDIIPSNGATFYNQKFKGQVTITADESTSTAYMELSSLRSEDTAVYYCARSHLLRASWFAYWGQGTLVTVSS

SEQ ID NO: 49 - protein, 113 aa, Artificial Sequence
  Description of Artificial Sequence Synthetic polypeptide
DIVMTQSPDSLAVSLGERATINCESSQSVLNSGNQKNYLTWYQQKPGQPPKLLIYWASTRESGVPDRFSGSGSGTDFTLTISSLQAEDVAVYYCQNDYSYPYTFGQGTKLEIK

SEQ ID NO: 50 - protein, 120 aa, Artificial Sequence
  Description of Artificial Sequence Synthetic polypeptide
QVQLVQSGAEVKKPGSSVKVSCKASGGTFSDYYMKWVRQAPGQGLEWMGDIIPSNGATFYNQKFKGRVTITADESTSTAYMELSSLRSEDTAVYYCARSHLLRASWFAYWGQGTLVTVSS

SEQ ID NO: 51 - protein, 113 aa, Artificial Sequence; identical to SEQ ID NO: 49
  Description of Artificial Sequence Synthetic polypeptide

SEQ ID NO: 52 - protein, 118 aa, Artificial Sequence
  Description of Artificial Sequence Synthetic polypeptide
EVQLLESGGGLVQPGGSLRLSCAVSDYSITSGYYWNWIRQAPGKKLEWMGYISYDGSNNYNPSLKNGRITISRDTSKNTFYLQMNSLRAEDTAVYYCARGEGFYFDSWGQGTLVTVSS

SEQ ID NO: 53 - protein, 113 aa, Artificial Sequence
  Description of Artificial Sequence Synthetic polypeptide
EIVLTQSPGTLSLSPGERATLSCKSSQSLFFGSTQKNYLAWYQQKPGQAPRLLIYWASTRESGIPDRFSGSGSGTDFTLTISRLEPEDFAVYYCQQYYNYPWTFGQGTKLEIK

SEQ ID NO: 54 - protein, 118 aa, Artificial Sequence; identical to SEQ ID NO: 52
  Description of Artificial Sequence Synthetic polypeptide

SEQ ID NO: 55 - protein, 113 aa, Artificial Sequence
  Description of Artificial Sequence Synthetic polypeptide
EIVLTQSPGTLSLSPGERATLSCRASQSVFFGSTQKNYLAWYQQKPGQAPRLLIYWASTRESGIPDRFSGSGSGTDFTLTISRLEPEDFAVYYCQQYYNYPWTFGQGTKLEIK

SEQ ID NO: 56 - protein, 120 aa, Artificial Sequence
  Description of Artificial Sequence Synthetic polypeptide
QVQLVQSGAEVKKPGASVKVSCKASGYTFTDYYMKWVRQAPGQGLEWMGDIIPSNGATFYAQKFQGRVTMTRDTSTSTVYMELSSLRSEDTAVYYCARSHLLRASWFAYWGQGTLVTVSS

SEQ ID NO: 57 - protein, 113 aa, Artificial Sequence
  Description of Artificial Sequence Synthetic polypeptide
DIVMTQSPDSLAVSLGERATINCKSSQSLLNSGNQKNYLTWYQQKPGQPPKLLIYWASTRESGVPDRFSGSGSGTDFTLTISSLQAEDVAVYYCQNDYSYPYTFGQGTKLEIK

SEQ ID NO: 58 - protein, 120 aa, Artificial Sequence
  Description of Artificial Sequence Synthetic polypeptide
QVQLVQSGAEVKKPGSSVKVSCKASGGTFSDYYMKWVRQAPGQGLEWMGDIIPSNGATFYAQKFQGRVTITADESTSTAYMELSSLRSEDTAVYYCARSHLLRASWFAYWGQGTLVTVSS

SEQ ID NO: 59 - protein, 113 aa, Artificial Sequence
  Description of Artificial Sequence Synthetic polypeptide
DIVMTQSPDSLAVSLGERATINCESSQSLLNSGNQKNYLTWYQQKPGQPPKLLIYWASTRESGVPDRFSGSGSGTDFTLTISSLQAEDVAVYYCQNDYSYPYTFGQGTKLEIK

SEQ ID NO: 60 - protein, 120 aa, Artificial Sequence; identical to SEQ ID NO: 58
  Description of Artificial Sequence Synthetic polypeptide

SEQ ID NO: 61 - protein, 113 aa, Artificial Sequence; identical to SEQ ID NO: 57
  Description of Artificial Sequence Synthetic polypeptide

SEQ ID NO: 62 - protein, 117 aa, Artificial Sequence
  Description of Artificial Sequence Synthetic polypeptide
QVQLQESGPGLVKPSQSLSLTCSVTDYSITSGYYWNWIRQFPGNKLEWMGYISYDGSNNYNPSLKNRISITRDTSKNQFFLKLSSVTTEDTATYYCSRGEGFYFDSWGQGTTLTVSS

SEQ ID NO: 63 - protein, 113 aa, Artificial Sequence
  Description of Artificial Sequence Synthetic polypeptide
DIMMSQSPSSLAVSVGEKFTMTCKSSQSLFFGSTQKNYLAWYQQKPGQSPKLLIYWASTRESGVPDRFTGSGSGTDFTLAISSVMPEDLAVYYCQQYYNYPWTFGGGTKLEIK

SEQ ID NO: 64 - protein, 120 aa, Artificial Sequence; identical to SEQ ID NO: 46
  Description of Artificial Sequence Synthetic polypeptide

SEQ ID NO: 65 - protein, 114 aa, Artificial Sequence; identical to SEQ ID NO: 47
  Description of Artificial Sequence Synthetic polypeptide

SEQ ID NO: 66 - protein, 119 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNSKNTLYLQMNSLRAEDTAVYYCARAEWFSEALDYWGQGTLVTVSS

SEQ ID NO: 67 - protein, 108 aa, Artificial Sequence
  Description of Artificial Sequence Synthetic polypeptide
QLTQPPSVSAAPGQKVTISCSGSNSNIGNNYVSWYQQLPGTAPKLLIYDNNRRPSGIPDRFSGSKSGTSATLGITGLQTGDEADYFCGTWDSSLSAGVFGGGTKLTVL

SEQ ID NO: 68 - protein, 118 aa, Artificial Sequence
  Description of Artificial Sequence Synthetic polypeptide
QVQLVQSGGGLVKPGGSLRLSCAASGFTFSSYSMNWVRQAPGKGLEWVSSISSSSSYIYYADSVKGRFTISRDNAKNSLYLQMNSLRPEDTAVYYCARESGSDALDIWGRGTMVTVSS

SEQ ID NO: 69 - protein, 111 aa, Artificial Sequence
  Description of Artificial Sequence Synthetic polypeptide
QSVVTQPPSVSAAPGQKVTISCSGSGSNIGNNYVSWYQQLPGTAPKLLIYDNNKRPSGIPDRFSGSKSGTSATLGITGLQTGDEADYYCATWDSSLSAPWVFGGGTKVTVL

SEQ ID NO: 70 - protein, 118 aa, Artificial Sequence
  Description of Artificial Sequence Synthetic polypeptide
EVQLVESGGGLVQPGGSLRLSCAASGFTFSSYEMNWVRQAPGKGLEWVSSISSSSSYIYYADSVKGRFTISRDNAKNSLYLQMNSLRAEDTAVYYCARADYYEAFDIWGQGTMVTVSS

SEQ ID NO: 71 - protein, 110 aa, Artificial Sequence
  Description of Artificial Sequence Synthetic polypeptide
NFMLTQPASVSGSPGQSITISCTGTSADVGGDYYVSWYQQHPGKAPKLTIYDVSERPSGVSNRFSGSKSGNTASLTISGLQTEDEADYYCGSYTSSGTWLFGGGTKLTVL

SEQ ID NO: 72 - protein, 119 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARAGTRGDAFDIWGQGTMVTVSS

SEQ ID NO: 73 - protein, 108 aa, Artificial Sequence
  Description of Artificial Sequence Synthetic polypeptide
QSALTQPPSVSVAPGQTARITCGGNNIGSKSVHWYQQKPGQAPVLVVYDDSDRPSGIPERFSGSNSGNTATLTISRVEAGDEADYYCQVWDSSSDHLVFGGGTKVTVL

SEQ ID NO: 74 - protein, 130 aa, Artificial Sequence
  Description of Artificial Sequence Synthetic polypeptide
QVQLVQSGAEVKKPGASVKVSCKASGYTFTSYGISWVRQAPGQGLEWMGWISAYNGNTKYSQKLRGRVTMTRDTSTSTAYMELRSLRSDDTAVYYCARDEEYDFWSGYGSWYYYYGMDVWGQGTTVTVSS

SEQ ID NO: 75 - protein, 109 aa, Artificial Sequence
  Description of Artificial Sequence Synthetic polypeptide
QLVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAVFGGGTKLTVL

SEQ ID NO: 76 - protein, 118 aa, Artificial Sequence
  Description of Artificial Sequence Synthetic polypeptide
EVQLVDSGGGLVKPGGSLRLSCAASGFTFSSYSMNWVRQAPGKGLEWVSSISSSSSYIYYADSVKGRFTISRDNAKNSLYLQMNSLRAEDTAVYYCAREDYYDSIDYWGQGTLVTVSS

SEQ ID NO: 77 - protein, 110 aa, Artificial Sequence
  Description of Artificial Sequence Synthetic polypeptide
QAGLTQPPSVSAAPGQQFTISCSGSSSNIGKNYVSWYQQLPGSAPKLLIYDNHKRPSGIPDRFSGSKSGTSATLGITGLQTGDEADYYCGTWDDSLSGWVFGGGTKLTVL

SEQ ID NO: 78 - protein, 120 aa, Artificial Sequence
  Description of Artificial Sequence Synthetic polypeptide
EVQLVESGGGLVQPGGSLRLSCAASGFTFSSYEMNWVRQAPGKGLEWVSYISSSGSTIYYADSVKGRFTISRDNAKNSLYLQMNSLRAEDTAVYYCARVQQWPDDAFDIWGQGTMVTVSS

SEQ ID NO: 79 - protein, 111 aa, Artificial Sequence
  Description of Artificial Sequence Synthetic polypeptide
QAGLTQPPSASGTPGQRVTIACSGSSSNIGTYTVNWYQHVPGTAPKLLIYSTYQRPLEVPDRFSGSKSGTSASLAISGLRSEDEGDYYCASWDDRLNGFYVFGSGTKVTVL

SEQ ID NO: 80 - protein, 118 aa, Artificial Sequence
  Description of Artificial Sequence Synthetic polypeptide
EVQLVQSGGGVVQPGRSLRVSCAASGFTFSSYGMHWVRQTPGKGLEWVAGIWYDENDKYYADSVKGRFTISRDNSKNTLHLQMNSLRAEDTAVYYCARQFRDYYFDVWGRGTLVTVSS

SEQ ID NO: 81 - protein, 111 aa, Artificial Sequence
  Description of Artificial Sequence Synthetic polypeptide
QSVLTQPRSVSGSPGQSVTISCTGTSIDVDKDNLVSWYQQHPGRVPKLIIYDVNKRPSGVPDHFSGSKSGTSASLAISGLRSEDEADYYCAAWDDSLSSWVFGGGTKVTVL

SEQ ID NO: 82 - protein, 121 aa, Artificial Sequence
  Description of Artificial Sequence Synthetic polypeptide
EVQLVQSGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTDYDFWSGYYYWGQGTTVTVSS

SEQ ID NO: 83 - protein, 111 aa, Artificial Sequence
  Description of Artificial Sequence Synthetic polypeptide
LPVLTQPASVSGSPGQSITISCTGTSSDVGRYDYVSWYQQHPGKAPQLMIYDVSNRPSGVSNRFSGSKSGNTASLTISGLQAEDEADYYCSSYTGSSTLYVFGTGTKVTVL

SEQ ID NO: 84 - protein, 122 aa, Artificial Sequence
  Description of Artificial Sequence Synthetic polypeptide
EVQLVQSGAEVKKPGSSVKVSCKASGGTFSTYAISWVRQAPGQGLEWMGGTIPKFGTANYAQKFQGRVTITADESTSTAYMELSSLRSEDTAVYYCARAVVPAAIVEAMDVWGQGTTVTVSS

SEQ ID NO: 85 - protein, 122 aa, Artificial Sequence; identical to SEQ ID NO: 84
  Description of Artificial Sequence Synthetic polypeptide

SEQ ID NO: 86 - protein, 126 aa, Artificial Sequence
  Description of Artificial Sequence Synthetic polypeptide
QVQLVQSGAEVKKPGASVKVSCKASGYTLSMYGISWVRHAPGQGLEWMGWINPYTGDRKYAQRFQGRLTVTTDTSTATSYMELTSLRSDDTAVYYCAREEYHDSMIGYYVGGFDLWGQGTLVTVSS

SEQ ID NO: 87 - protein, 108 aa, Artificial Sequence
  Description of Artificial Sequence Synthetic polypeptide
QAVLTQPPSVSVAPGKTARITCGGNNIGSKSVHWYQQKPGQAPVLVVYDDSDRPSGIPERFSGSNSGNTATLTISRVEAGDEADYYCQVWDSSSDHVVFGGGPQLTVL

SEQ ID NO: 88 - protein, 117 aa, Artificial Sequence
  Description of Artificial Sequence Synthetic polypeptide
EVQLLESGGGLVKPGGSLRLSCAASGFTFSSYSMNWVRQAPGKGLEWVSSISSSSSYIYYADSVKGRFTISRDNAKNSLYLQMNSLRAEDTAVYYCARANWDAFDIWGQGTMVTVSS

SEQ ID NO: 89 - protein, 110 aa, Artificial Sequence
  Description of Artificial Sequence Synthetic polypeptide
QSVLTQPPSVSAAPGHEVTISCSGSSSNIGNNYVSWYQQVPGTAPKLLIYDNNKRASEIPDRFFGSKSGTSATLGVSGLQTGDEADYYCGTWDSSLNDVVFGGGTKLTVL

SEQ ID NO: 90 - protein, 121 aa, Artificial Sequence
  Description of Artificial Sequence Synthetic polypeptide
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTDYDFWSGYYYWGQGTLVTVSS

SEQ ID NO: 91 - protein, 111 aa, Artificial Sequence
  Description of Artificial Sequence Synthetic polypeptide
LPVLTQSASVSGSPGQSITISCTGTSSDVGRYDYVSWYQQHPGKAPQLMIYDVSNRPSGVSNRFSGSKSGNTASLTISGLQAEDEADYYCSSYTGSSTLYVFGTGTKVTVL

SEQ ID NO: 92 - protein, 124 aa, Artificial Sequence
  Description of Artificial Sequence Synthetic polypeptide
EVQLVQSGAEVKKPGASVKVSCKASGYTFTGYYMHWVRQAPGQGLEWMGWISAYNGNTNYAQKLQGRVTMTTDTSTSTAYMELRSLRSDDTAVYYCAREEDYYGSGEHYYFDYWGQGTLVTVSS

SEQ ID NO: 93 - protein, 107 aa, Artificial Sequence
  Description of Artificial Sequence Synthetic polypeptide
DIQLTQSPSSLSASVGDRVTITCRASQSISDYLNWYHQKPGKAPRLLIYAASSLQSGVPSRFSGTRSGTDFTLTINNLQPEDSATYYCQQSYSTPLTFGGGTKVDIK

SEQ ID NO: 94 - protein, 121 aa, Artificial Sequence
  Description of Artificial Sequence Synthetic polypeptide
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDMNILATVPFDIWGQGTMVTVSS

SEQ ID NO: 95 - protein, 107 aa, Artificial Sequence
  Description of Artificial Sequence Synthetic polypeptide
DIQMTQSPSSLSASVGDRVTITCRASQSISTYLNWYQQKPGKAPNLLIYAAFSLQSGVPSRFSGSGSGTDFTLTINSLQPEDFATYYCQQGDSVPLTFGGGTKLEIK

SEQ ID NO: 96 - protein, 121 aa, Artificial Sequence
  Description of Artificial Sequence Synthetic polypeptide
QVQLVQSGAEVKKPGASVKVSCKASGYTFTGYYMHWVRQAPGQGLEWMGWINPNSGGTNYAQKFQGRVTLTRDTSISTVYMELSRLRSDDTAVYYCARDMNILATVPFDIWGQGTMVTVSS

SEQ ID NO: 97 - protein, 107 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTVNSLQPEDFATYYCQQGDSVPLTFGGGTRLEIK

SEQ ID NO: 98 - protein, 121 aa, Artificial Sequence
  Description of Artificial Sequence Synthetic polypeptide
QVQLVQSGAEVKKPGASVKVSCKASGYIFTGYYIHWVRQAPGQGLEWMGWINPNSGGTNYAQKFQGRVTMTRDTSISTAYMELSGLRSDDPAVYYCARDMNILATVPFDIWGQGTLVTVSS

SEQ ID NO: 99 - protein, 107 aa, Artificial Sequence
  Description of Artificial Sequence Synthetic polypeptide
DIQLTQSPSSLSASVGDRVTITCRASQSISSYLNWYQQKPGKAPKLLIYAASSLQSGVPSRFSGSGSGTDFTLTVNSLQPEDFATYYCQQGDSVPLTFGGGTKVEIK

SEQ ID NO: 100 - protein, 121 aa, Artificial Sequence
  Description of Artificial Sequence Synthetic polypeptide
QVQLQQSGAEVKKSGASVKVSCKASGYTFTDYYMHWLRQAPGQGLEWMGWINPNSGDTNYAQKFQGRVTLTRDTSISTVYMELSRLRSDDTAVYYCARDMNILATVPFDIWGQGTMVTVSS

SEQ ID NO: 101 - protein, 107 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQGDSVPLTFGGGTKVEIK

SEQ ID NO: 102 - protein, 120 aa, Artificial Sequence [sequence omitted]

SEQ ID NO: 103 - protein, 114 aa, Artificial Sequence
  Description of Artificial Sequence Synthetic polypeptide
DIVMTQSPDSLAVSLGERATINCESSQSLLNSGNQKNYLTWYQQKPGQPPKPLIYWASTRESGVPDRFSGSGSGTDFTLTISSLQAEDVAVYYCQNDYSYPYTFGQGTKLEIKR

SEQ ID NO: 104 - protein, 449 aa, Artificial Sequence
  Description of Artificial Sequence Synthetic polypeptide
QVQLQQSGAEVKKPGASVKVSCKASGYTFTDYYMKWVKQSHGKSLEWMGDIIPSNGATFYNQKFKGKATLTVDRSTSTAYMELSSLRSEDTAVYYCARSHLLRASWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 105 - protein, 220 aa, Artificial Sequence
  Description of Artificial Sequence Synthetic polypeptide
DFVMTQSPDSLAVSLGERATINCKSSQSLLNTGNQKNYLTWYQQKPGQPPKLLIYWASTRESGVPDRFTGSGSGTDFTLTISSLQAEDVAVYYCQNDYSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 485 aa, Artificial Sequence
  Description of Artificial Sequence Synthetic polypeptide
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 120 aa, Artificial Sequence
  Description of Artificial Sequence Synthetic polypeptide
QVQLVQSGAELKKPGASVKVSCKASGYTFTDYYMKWVRQAPGQGLEWIGDIIPSNGATFYNQKFKGRVTITVDKSTSTAYMELSSLRSEDTAVYYCARSHLLRASWFAYWGQGTLVTVSS

SEQ ID NO: 108 - protein, 113 aa, Artificial Sequence
  Description of Artificial Sequence Synthetic polypeptide
DFVMTQSPDSLAVSLGERVTMSCKSSQSLLNSGNQKNYLTWYQQKPGQPPKLLIYWASTRESGVPDRFSGSGSGTDFTLTISSLQAEDVAVYYCQNDYSYPYTFGQGTKLEIK

SEQ ID NO: 109 - protein, 120 aa, Artificial Sequence
  Description of Artificial Sequence Synthetic polypeptide
EVQLVQSGAELKKPGASVKVSCKASGYTFTDYYMKWVRQAPGQGLEWIGDIIPSNGATFYNQKFKGRVTITVDKSTSTAYMELSSLRSEDTAVYYCARSHLLRASWFAYWGQGTLVTVSS

SEQ ID NO: 110 - protein, 113 aa, Artificial Sequence; identical to SEQ ID NO: 108
  Description of Artificial Sequence Synthetic polypeptide

SEQ ID NO: 111 - protein, 115 aa, Artificial Sequence; identical to SEQ ID NO: 44
  Description of Artificial Sequence Synthetic polypeptide

SEQ ID NO: 112 - protein, 107 aa, Artificial Sequence; identical to SEQ ID NO: 45
  Description of Artificial Sequence Synthetic polypeptide

SEQ ID NO: 113 - protein, 118 aa, Artificial Sequence
  Description of Artificial Sequence Synthetic polypeptide
QIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSS

SEQ ID NO: 114 - protein, 111 aa, Artificial Sequence
  Description of Artificial Sequence Synthetic polypeptide
DIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELK

SEQ ID NO: 115 - protein, 120 aa, Artificial Sequence
  Description of Artificial Sequence Synthetic polypeptide
EVQLQQSGPELVKPGASVKMSCKASGYTFTDYYMKWVKQSHGKSLEWIGDIIPSNGATFYNQKFKGKATLTVDRSSSTAYMHLNSLTSEDSAVYYCTRSHLLRASWFAYWGQGTLVTVSA

SEQ ID NO: 116 - protein, 114 aa, Artificial Sequence
  Description of Artificial Sequence Synthetic polypeptide
DFVMTQSPSSLTVTAGEKVTMSCKSSQSLLNSGNQKNYLTWYLQKPGQPPKLLIYWASTRESGVPDRFTGSGSGTDFTLTISSVQAEDLAVYYCQNDYSYPYTFGGGTKLEIKR

SEQ ID NO: 117 - protein, 121 aa, Artificial Sequence
  Description of Artificial Sequence Synthetic polypeptide
QVQLVQSGAEVKKPGASVKVSCKASGYIFTSSIMHWVRQAPGQGLEWIGYIKPYNDGTKYNEKFKGRATLTSDRSTSTAYMELSSLRSEDTAVYYCAREGGNDYYDTMDYWGQGTLVTVSS

SEQ ID NO: 118 - protein, 108 aa, Artificial Sequence
  Description of Artificial Sequence Synthetic polypeptide
DIQMTQSPSSLSASVGDRVTITCRASQDINSYLSWFQQKPGKAPKTLIYRVNRLVDGVPSRFSGSGSGNDYTLTISSLQPEDFATYYCLQYDAFPYTFGQGTKVEIKR

SEQ ID NO: 119 - protein, 125 aa, Artificial Sequence
  Description of Artificial Sequence Synthetic polypeptide
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSS

SEQ ID NO: 120 - protein, 109 aa, Artificial Sequence
  Description of Artificial Sequence Synthetic polypeptide
QTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVL

SEQ ID NO: 121 - protein, 125 aa, Artificial Sequence; identical to SEQ ID NO: 119
  Description of Artificial Sequence Synthetic polypeptide

SEQ ID NO: 122 - protein, 109 aa, Artificial Sequence
  Description of Artificial Sequence Synthetic polypeptide
QTVVTQEPSLSVSPGGTVTLTCGSSTGAVTTSNYANWVQQTPGQAPRGLIGGTDKRAPGVPDRFSGSLLGDKAALTITGAQAEDEADYYCALWYSDLWVFGGGTKLTVL

SEQ ID NO: 123 - protein, 119 aa, Artificial Sequence
  Description of Artificial Sequence Synthetic polypeptide
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSS

SEQ ID NO: 124 - protein, 112 aa, Artificial Sequence
  Description of Artificial Sequence Synthetic polypeptide
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIK

SEQ ID NO: 125 - protein, 119 aa, Artificial Sequence
  Description of Artificial Sequence Synthetic polypeptide
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGRYFYDVWGQGTLVTVSS

SEQ ID NO: 126 - protein, 112 aa, Artificial Sequence
  Description of Artificial Sequence Synthetic polypeptide
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYFRRTFGGGTKVEIK

SEQ ID NO: 127 - protein, 119 aa, Artificial Sequence
  Description of Artificial Sequence Synthetic polypeptide
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGQYFYDVWGQGTLVTVSS

SEQ ID NO: 128 - protein, 112 aa, Artificial Sequence
  Description of Artificial Sequence Synthetic polypeptide
DIVMTQSPDSLAVSLGERATINCKSSQSLLNARTGKNYLAWYQQKPGQPPKLLIYWASTRESGVPDRFSGSGSGTDFTLTISSLQAEDVAVYYCTQSYFRRTFGGGTKVEIK

SEQ ID NO: 129 - protein, 737 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVAASQAALGL

SEQ ID NO: 130 - protein, 5 aa, Artificial Sequence
  Description of Artificial Sequence Synthetic peptide
TYAMN

SEQ ID NO: 131 - protein, 5 aa, Artificial Sequence
  Description of Artificial Sequence Synthetic peptide
DYYMH

SEQ ID NO: 132 - protein, 19 aa, Artificial Sequence
  Description of Artificial Sequence Synthetic peptide
RIRSKYNNYATYYADSVKD

SEQ ID NO: 133 - protein, 17 aa, Artificial Sequence
  Description of Artificial Sequence Synthetic peptide
WIDLENANTIYDAKFQG

SEQ ID NO: 134 - protein, 17 aa, Artificial Sequence
  Description of Artificial Sequence Synthetic peptide
WIDLENANTVYDAKFQG

SEQ ID NO: 135 - protein, 14 aa, Artificial Sequence
  Description of Artificial Sequence Synthetic peptide
HANFGAGYVSWFAH

SEQ ID NO: 136 - protein, 10 aa, Artificial Sequence
  Description of Artificial Sequence Synthetic peptide
DAYGRYFYDV

SEQ ID NO: 137 - protein, 10 aa, Artificial Sequence
  Description of Artificial Sequence Synthetic peptide
DAYGQYFYDV

SEQ ID NO: 138 - protein, 14 aa, Artificial Sequence
  Description of Artificial Sequence Synthetic peptide
GSSTGAVTTSNYAN

SEQ ID NO: 139 - protein, 17 aa, Artificial Sequence
  Description of Artificial Sequence Synthetic peptide
KSSQSLLNARTGKNYLA

SEQ ID NO: 140 - protein, 7 aa, Artificial Sequence
  Description of Artificial Sequence Synthetic peptide
GTDKRAP

SEQ ID NO: 141 - protein, 7 aa, Artificial Sequence
  Description of Artificial Sequence Synthetic peptide
WASTRES

SEQ ID NO: 142 - protein, 9 aa, Artificial Sequence
  Description of Artificial Sequence Synthetic peptide
ALWYSNHWV

SEQ ID NO: 143 - protein, 9 aa, Artificial Sequence
  Description of Artificial Sequence Synthetic peptide
ALWYSDLWV

SEQ ID NO: 144 - protein, 8 aa, Artificial Sequence
  Description of Artificial Sequence Synthetic peptide
KQSYSRRT

SEQ ID NO: 145 - protein, 8 aa, Artificial Sequence
  Description of Artificial Sequence Synthetic peptide
KQSYFRRT

SEQ ID NO: 146 - protein, 8 aa, Artificial Sequence
  Description of Artificial Sequence Synthetic peptide
TQSYFRRT